SEQUENCE LISTING (184 sequences)

SEQ ID NO: 1 - protein, 595 aa, Homo sapiens [sequence omitted]

SEQ ID NO: 2 - protein, 314 aa, Homo sapiens
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 3 - protein, 595 aa, Artificial Sequence
  Estrogen Receptor with G521R mutation
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 4 - protein, 314 aa, Artificial Sequence
  EBD with G521R mutation
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 5 - protein, 595 aa, Artificial Sequence
  Estrogen Receptor with E353A mutation
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRALVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 6 - protein, 314 aa, Artificial Sequence
  EBD (E353A) Pairs with estrogen analog ES8
SAGDMRAANLWPSPLMIKRSKKNSLALSLTADQMVSALLDAEPPILYSEYDPTRPFSEASMMGLLTNLADRALVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 7 - DNA, 942 bp, Artificial Sequence
  EBD (E353A) Coding Sequence
tctgctggcgatatgagagccgctaacctgtggccttctccactgatgatcaagcggagcaagaagaactctctggccctgtctctgaccgccgaccagatggtttctgctctgctggatgccgagcctcctatcctgtactccgagtacgaccccaccagaccttttagcgaggccagcatgatgggcctgctgaccaatctggctgacagagccctggtgcacatgatcaactgggccaagcgcgtgcccggctttgtcgatctgacactgcacgaccaagtgcatctgctggaatgcgcctggctggaaattctgatgatcggcctcgtgtggcggagcatggaacaccctggaaaactgctgttcgcccctaacctgctgctcgacagaaaccagggcaaatgcgtggaaggcatggtggaaatcttcgatatgctgctggccacctccagccggttccggatgatgaatctccagggcgaagagttcgtgtgcctgaagtccatcatcctgctgaatagcggcgtgtacacattcctgagcagcaccctgaaatccctggaagagaaggaccacatccaccgggtgctcgacaagatcaccgacacactgatccacctgatggccaaggccggactgactctgcagcagcaacatcagagactggcccagctgctgctcatcctgagccacatcagacacatgagcaacaaaggcatggaacatctgtacagcatgaagtgcaagaacgtggtgcccctgtacgacctgctcctggaaatgctggacgcccacagactgcacgcccctacatctagaggtggcgcctctgtggaagagacagaccagtctcacctggccacagccggcagcacaagctctcattccctgcagaagtactacatcaccggcgaggccgagggatttcctgccacagtt

SEQ ID NO: 8 - protein, 595 aa, Artificial Sequence
  Estrogen Receptor with L384M and M421G mutations
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 9 - protein, 314 aa, Artificial Sequence
  EBD with L384M and M421G mutations
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 10 - protein, 595 aa, Artificial Sequence
  Estrogen Receptor with L384M, M421G, and G521R mutations
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 11 - protein, 314 aa, Artificial Sequence
  EBD (L384M, M421G, G521R) Pairs with estrogen analog CMP8
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKRMEHLYSMKCKNVVPLYDLLLEMLDAHRLHAPTSRGGASVEETDQSHLATAGSTSSHSLQKYYITGEAEGFPATV

SEQ ID NO: 12 - protein, 595 aa, Artificial Sequence
  Estrogen Receptor with G400V, M543A, and L544A mutations
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 13 - protein, 314 aa, Artificial Sequence
  EBD with G400V, M543A, and L544A mutations (ER(T2))
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 14 - DNA, 942 bp, Artificial Sequence
  Alternative G400V, M543A, and L544A ER(T2) Coding Sequence
tcagcgggtgacatgcgagctgccaatctctggccgtcacctttgatgattaaacgatcaaagaaaaatagcctggctctgagcttgacggctgatcaaatggtaagtgcactgctcgacgccgaaccgccgatcctgtattccgagtatgaccccacacggccattctccgaagccagtatgatgggacttcttaccaaccttgcagatcgcgaactcgtacatatgataaattgggcgaagcgcgttccagggttcgtcgacctcacgttgcatgatcaggtccatctgctggaatgcgcgtggttggaaatcttgatgattggtttggtctggcgatctatggagcatccggtgaaacttcttttcgcgcccaatcttcttttggatcgcaatcaagggaaatgcgtggaaggaatggtcgaaatttttgatatgcttctcgcgaccagctcccgattcaggatgatgaatttgcagggtgaagagtttgtatgtcttaagtctattatattgctcaactccggcgtttatacattcctttctagcacacttaaatcactcgaggaaaaggaccacatccaccgggttttggataaaatcactgatacactgattcatttgatggccaaggccggactcacactccaacaacagcaccaacggcttgcacagctcctcctcatactttctcatataaggcacatgtcaaataagggaatggagcacctgtactccatgaagtgtaaaaatgtagtccctctgtatgatctgctccttgaggctgcagacgcgcatagactgcacgccccaacgtcacgagggggggcctcagtggaagagacagatcagagccatctcgcaaccgcaggatctacatccagccactctcttcagaaatactatataactggcgaagccgaagggttccctgccaccgtt

SEQ ID NO: 15 - DNA, 942 bp, Artificial Sequence
  EBD (L384M, M421G, G521R) Coding Sequence
tctgctggcgatatgagagccgctaacctgtggccttctccactgatgatcaagcggagcaagaagaactctctggccctgtctctgaccgccgaccagatggtttctgctctgctggatgccgagcctcctatcctgtactccgagtacgaccccaccagaccttttagcgaggccagcatgatgggcctgctgaccaatctggccgacagagaactggtgcacatgatcaactgggccaagcgcgtgcccggctttgtcgatctgacactgcacgaccaagtgcatctgctggaatgcgcctggatggaaatcctgatgatcggcctcgtgtggcggagcatggaacaccctggaaaactgctgttcgcccctaacctgctgctcgacagaaaccagggcaaatgcgtggaaggcggcgtggaaatcttcgatatgctgctggccacctccagccggttccggatgatgaatctccagggcgaagagttcgtgtgcctgaagtccatcatcctgctgaatagcggcgtgtacacattcctgagcagcaccctgaaatccctggaagagaaggaccacatccaccgggtgctcgacaagatcaccgacacactgatccacctgatggccaaggccggactgactctgcagcagcaacatcagagactggcccagctgctgctcatcctgagccacatcagacacatgagcaacaagcggatggaacatctgtactccatgaagtgcaagaacgtggtgcccctgtacgacctgctcctggaaatgctggacgcccacagactgcacgcccctacatctagaggtggcgcctctgtggaagagacagaccagtctcacctggccacagccggcagcacaagctctcattccctgcagaagtactacatcaccggcgaggccgagggctttccagccacagtt

SEQ ID NO: 16 - protein, 264 aa, Artificial Sequence
  Epidermal growth factor receptor (EGFR) (806) single chain variable (scFv) antibody fragment
MLLLVTSLLLCELPHPAFLLIPDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGSTSGSGKPGSGEGSTKGDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 17 - DNA, 787 bp, Artificial Sequence
  EGFRVIII (806) scFv Coding Sequence
atgctgctgctggtgaccagcctgctgctgtgcgagctgccccaccccgcctttctgctgatcccccaggtgcagctgcagcagcctggcgccgagctggtgaagccaggcgccagcgtgaagctgtcctgcaaggccagcggctacaccttcaccggctactggatgcactgggtgaagcagagacccggccacggcctggaatggatcggcgagatcaaccccagcaacggccggaccaactacaacgagcggttcaagagcaaggccaccctgaccgtggacaagagcagcaccaccgccttcatgcagctgtccggcctgaccagcgaggacagcgccgtgtacttctgcgccagggactactacggcaccagctacaacttcgactactggggccagggcaccacactgaccgtgagcagcggcggagggggctctggcggcggaggatctgggggagggggcagcgacatccagatgacccagagcagcagcagcttcagcgtgagcctgggcgaccgggtgaccatcacctgtaaggccaacgaggacatcaacaaccggctggcctggtatcagcagacccccggcaacagccccaggctgctgatcagcggcgccaccaacctggtgaccggcgtgcccagccggtttagcggcagcggctccggcaaggactacaccctgaccatcacaagcctgcaggccgaggacttcgccacctactactgccagcagtactggtccacccccttcaccttcggcagcggcaccgagctggaaa

SEQ ID NO: 18 - protein, 265 aa, Artificial Sequence
  EGFR scFv
MLLLVTSLLLCELPHPAFLLIPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRT

SEQ ID NO: 19 - protein, 268 aa, Artificial Sequence
  Human CD19 (huCD19) (G01S) scFv
MLLLVTSLLLCELPHPAFLLIPEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVL

SEQ ID NO: 20 - protein, 267 aa, Artificial Sequence
  Mouse CD19 (muCD19) (FMC63) scFv
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 21 - protein, 244 aa, Artificial Sequence
  CD19 scFv
DIQMTQTTSSLSALGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 22 - DNA, 735 bp, Artificial Sequence
  CD19 scFv Coding Sequence
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 23 - protein, 266 aa, Artificial Sequence
  CD20 (Leu 16) scFv
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 24 - DNA, 798 bp, Artificial Sequence
  CD20 scFv Coding Sequence
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctca

SEQ ID NO: 25 - protein, 270 aa, Artificial Sequence
  CD22 (m971) scFv
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKRT

SEQ ID NO: 26 - protein, 316 aa, Artificial Sequence
  B7H3 (hBRCA84D) scFv
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITGQPMTFPPEALWVTVGLSVCLIALLVALAFVCWRKIKQSCEEENAGAEDQDGEGEGSKTALQPLKHSDSKEDDGQEIA

SEQ ID NO: 27 - protein, 264 aa, Artificial Sequence
  L1CAM (CE7) scFv
MLLLVTSLLLCELPHPAFLLIPQVQLQQPGAELVKPGASVKLSCKASGYTFTGYWMHWVKQRPGHGLEWIGEINPSNGRTNYNERFKSKATLTVDKSSTTAFMQLSGLTSEDSAVYFCARDYYGTSYNFDYWGQGTTLTVSSGGGGSGGGGSGGGGSDIQMTQSSSSFSVSLGDRVTITCKANEDINNRLAWYQQTPGNSPRLLISGATNLVTGVPSRFSGSGSGKDYTLTITSLQAEDFATYYCQQYWSTPFTFGSGTELEIK

SEQ ID NO: 28 - protein, 259 aa, Artificial Sequence
  EphA2 (2A4) scFv
MLLLVTSLLLCELPHPAFLLIPQVQLLESGGGLVQPGGSLRLSCAASGFTFSSYTMSWVRQAPGQALEWMGTISSRGTYTYYPDSVKGRFTISRDNAKNSLYLQMNSLRAEDTAVYYCAREAIFTHWGRGTLVTVSSGGGGSGGGGSGGGGSDIQLTQSPSSLSASVGDRVTITCKASQDINNYHSWYQQKPGQAPRLLIYRANRLVDGVPDRFSGSGYGTDFTLTINNIESEDAAYYFCLKYNVFPYTFGQGTKVEIK

SEQ ID NO: 29 - protein, 259 aa, Artificial Sequence
  EpHA2 (4H5) scFv
MLLLVTSLLLCELPHPAFLLIPQVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTVSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIK

SEQ ID NO: 30 - protein, 273 aa, Artificial Sequence
  FITC (E2) scFv
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVS

SEQ ID NO: 31 - protein, 127 aa, Artificial Sequence
  GD2 (hu3F8) scFv
MLLLVTSLLLCELPHPAFLLIPSIVMTQTPKFLLVSAGDRVTITCKASQSVSNDVTWYQQKAGQSPKLLIYSASNRYSGVPDRFTGSGYGTAFTFTISTVQAEDLAVYFCQQDYSSFGGGTKLEIKR

SEQ ID NO: 32 - protein, 267 aa, Artificial Sequence
  Her2 (Herceptin) scFv
MLLLVTSLLLCELPHPAFLLIPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 33 - protein, 240 aa, Artificial Sequence
  IL13Ra2 (hu08) VlVh scFv
MLLLVTSLLLCELPHPAFLLIPDIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIKGGGGSGGGGSGGGGSEVQLVESGGGLVQPGGSLRLSCAASGFTFSRNGMSWVRQAPGKGLEWVATVSSGGSYIYYADSVKGRFTISRDNAKNSLYLQMNSLRAEDTAVYYC

SEQ ID NO: 34 - protein, 269 aa, Artificial Sequence
  IL13Ra2 hu08 VhVl scFv
MLLLVTSLLLCELPHPAFLLIPEVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSSGGGGSGGGGSGGGGSDIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIKESK

SEQ ID NO: 35 - protein, 264 aa, Artificial Sequence
  IL13Ra2 (hu07) VhVl scFv
MLLLVTSLLLCELPHPAFLLIPDIQMTQSPSSLSASVGDRVTITCTASLSVSSTYLHWYQQKPGKAPKLLIYSTSNLASGVPSRFSGSGSGTDFTLTISSLQPEDFATYYCHQYHRSPLTFGGGTKVEIKGGGGSGGGGSGGGGSEVQLVESGGGLVQPGGSLRLSCAASGFTFTKYGVHWVRQAPGKGLEWVAVKWAGGSTDYNSALMSRFTISRDNAKNSLYLQMNSLRAEDTAVYYCARDHRDAMDYWGQGTLVTVSSESK

SEQ ID NO: 36 - protein, 264 aa, Artificial Sequence
  IL13Ra2 (hu07) VhVl scFv
MLLLVTSLLLCELPHPAFLLIPEVQLVESGGGLVQPGGSLRLSCAASGFTFTKYGVHWVRQAPGKGLEWVAVKWAGGSTDYNSALMSRFTISRDNAKNSLYLQMNSLRAEDTAVYYCARDHRDAMDYWGQGTLVTVSSGGGGSGGGGSGGGGSDIQMTQSPSSLSASVGDRVTITCTASLSVSSTYLHWYQQKPGKAPKLLIYSTSNLASGVPSRFSGSGSGTDFTLTISSLQPEDFATYYCHQYHRSPLTFGGGTKVEIKESK

SEQ ID NO: 37 - protein, 269 aa, Artificial Sequence
  oaGD2 (8B6) VlVh scFv
MLLLVTSLLLCELPHPAFLLIPDVVMTQTPLSLPVSLGDQASISCRSSQSLLKNNGNTFLHWYLQKSGQSPKLLIYKVSNRLSGVPDRFSGSGSGTYFTLKISRVEAEDLGVYFCSQSTHIPYTFGGGTKLELKRGGGGSGGGGSGGGGSEVKLVESGGGLVLPGDSLRLSCATSEFTFTDYYMTWVRQPPRKALEWLGFIRNRANGYTTEYNPSVKGRFTISRDNSQSILYLQMNTLRTEDSATYYCARVSNWAFDYWGQGTTLTVSS

SEQ ID NO: 38 - protein, 270 aa, Artificial Sequence
  ROR1 (R12) scFv
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 39 - protein, 139 aa, Artificial Sequence
  CD33 (h2H12) VhVl scFv
MLLLVTSLLLCELPHPAFLLIPQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 40 - protein, 129 aa, Artificial Sequence
  CD33 (h2H12) VlVh scFv
MLLLVTSLLLCELPHPAFLLIPDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 41 - protein, 150 aa, Artificial Sequence
  Mesothelin (P4) scFv
MLLLVTSLLLCELPHPAFLLIPQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGS

SEQ ID NO: 42 - protein, 448 aa, Artificial Sequence
  VAR2CSA (ID1-DBL2Xb) scFv
MLLLVTSLLLCELPHPAFLLIPSLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKY

SEQ ID NO: 43 - protein, 133 aa, Artificial Sequence
  Anti-IL13Ra2 (IL13 zetakine) amino acid
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRF

SEQ ID NO: 44 - protein, 251 aa, Artificial Sequence
  FITCE2 scFv
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVS

SEQ ID NO: 45 - protein, 251 aa, Artificial Sequence
  FITCE2 TyrH133Ala scFv
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFASYMDVWGQGTLVTVS

SEQ ID NO: 46 - protein, 250 aa, Artificial Sequence
  FITCE2 HisH131Ala scFv
VLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGAFYSYMDVWGQGTLVTVS

SEQ ID NO: 47 - protein, 254 aa, Artificial Sequence
  FL (4M5.3) scFv
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 48 - protein, 265 aa, Artificial Sequence
  FL (4D5Flu) scFv
DYKDIQMTQSPSSLSASVGDRVTITCRASQSLVHSQGNTYLRWYQQKPGKAPKVLIYKVSNRFSGVPSRFSGSGSGTDFTLTISSLQPEDFATYYCQQSTHVPWTFGQGTKVELKRAGGGGSGGGGSGGGGSSGGGSGGGGSGGGGSEVQLVESGGGLVQPGGSLRLSCAASGFTFSDYWMNWVRQAPGKGLEWVAQIRNKPYNYETYYADSVKGRFTISRDTSKNTVYLQMNSLRAEDTAVYYCTGSYYGMDYWGQGTLVTVSS

SEQ ID NO: 49 - protein, 244 aa, Artificial Sequence
  FL (4420) scFv
DVVMTQTPLSLPVSLGDQASISCRSSQSLVHSQGNTYLRWYLQKPGQSPKVLIYKVSNRFSGVPDRFSGSGSGTDFTLKISRVEAEDLGVYFCSQSTHVPWTFGGGTKLEIGGGGSGGGGSGGGGSEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 50 - DNA, 789 bp, Artificial Sequence
  DNP scFv (VH to VL from rabbit)
cagtgtcagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtctcctcaggcggagggggctctggcggcggaggatctgggggagggggcagcccaggtgccacatttgcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttcggcggagggaccgaggtggtggtcagaggt

SEQ ID NO: 51 - protein, 263 aa, Artificial Sequence
  DNP scFv (VH to VL from rabbit) protein
QCQQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIVSSGGGGSGGGGSGGGGSPGATFAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRG

SEQ ID NO: 52 - DNA, 789 bp, Artificial Sequence
  DNP scFv (VL to VH from rabbit)
ccaggtgccacatttgcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttcggcggagggaccgaggtggtggtcagaggtggcggagggggctctggcggcggaggatctgggggagggggcagccagtgtcagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtctcctca

SEQ ID NO: 53 - protein, 263 aa, Artificial Sequence
  DNP scFv (VL to VH from rabbit) protein
PGATFAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRGGGGGSGGGGSGGGGSQCQQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIVSS

SEQ ID NO: 54 - protein, 217 aa, Artificial Sequence
  VH of DNP scFv (Ab-1; 1BAF)
DVQLQESGPGLVKPSQSQSLTCTVTGYSITSDYAWNWIRQFPGNKLEWMGYMSYSGSTRYNPSLRSRISITRDTSKNQFFLQLKSVTTEDTATYFCARGWPLAYWGQGTQVSVSEAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDC

SEQ ID NO: 55 - protein, 214 aa, Artificial Sequence
  VL of DNP scFv (Ab-1; 1BAF)
QIVLTQSPAIMSASPGEKVTMTCSASSSVYYMYWYQQKPGSSPRLLIYDTSNLASGVPVRFSGSGSGTSYSLTISRMEAEDAATYYCQQWSSYPPITFGVGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 56 - protein, 125 aa, Artificial Sequence
  VH of DNP scFv (Ab-2; XC)
QQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIV

SEQ ID NO: 57 - protein, 104 aa, Artificial Sequence
  VL of DNP scFv (Ab-2; XC)
AQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAF

SEQ ID NO: 58 - protein, 125 aa, Artificial Sequence; identical to SEQ ID NO: 56
  VH of DNP scFv (Ab-3)

SEQ ID NO: 59 - protein, 119 aa, Artificial Sequence
  VL of DNP scFv (Ab-3)
PGATFAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRG

SEQ ID NO: 60 - protein, 263 aa, Artificial Sequence; identical to SEQ ID NO: 53
  VL-linker-VH of DNP scFv (Ab-3)

SEQ ID NO: 61 - protein, 263 aa, Artificial Sequence; identical to SEQ ID NO: 51
  VH-linker-VL of DNP scFv (Ab-3)

SEQ ID NO: 62 - protein, 446 aa, Artificial Sequence
  VH-linker-VL of DNP scFv (1BAF)
DVQLQESGPGLVKPSQSQSLTCTVTGYSITSDYAWNWIRQFPGNKLEWMGYMSYSGSTRYNPSLRSRISITRDTSKNQFFLQLKSVTTEDTATYFCARGWPLAYWGQGTQVSVSEAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGGGGSGGGGSGGGGSQIVLTQSPAIMSASPGEKVTMTCSASSSVYYMYWYQQKPGSSPRLLIYDTSNLASGVPVRFSGSGSGTSYSLTISRMEAEDAATYYCQQWSSYPPITFGVGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 63 - protein, 446 aa, Artificial Sequence
  VL-linker-VH of DNP scFv (1BAF)
QIVLTQSPAIMSASPGEKVTMTCSASSSVYYMYWYQQKPGSSPRLLIYDTSNLASGVPVRFSGSGSGTSYSLTISRMEAEDAATYYCQQWSSYPPITFGVGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSDVQLQESGPGLVKPSQSQSLTCTVTGYSITSDYAWNWIRQFPGNKLEWMGYMSYSGSTRYNPSLRSRISITRDTSKNQFFLQLKSVTTEDTATYFCARGWPLAYWGQGTQVSVSEAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDC

SEQ ID NO: 64 - protein, 246 aa, Artificial Sequence
  VH-linker-VL of DNP scFv (XC)
QQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIVSSGGGGSGGGGSGGGGSAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAF

SEQ ID NO: 65 - protein, 254 aa, Artificial Sequence
  VL-linker-VH of DNP scFv (XC)
AQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRGGGGGSGGGGSGGGGSQQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIV

SEQ ID NO: 66 - DNA, 1338 bp, Artificial Sequence
  VH-linker-VL of DNP scFv (1BAF) coding sequence
gacgtacaactgcaagaatcaggtccgggactggtcaaacctagccagtctcagtccttgacgtgcaccgtgacgggctatagtataacgagtgattacgcttggaattggattcggcagtttccaggcaataagcttgagtggatgggctatatgtcttactccggctcaactaggtataatccgagccttcggtctcggatttctatcacaagagatacgagtaagaatcagttttttcttcaactcaaaagcgtcaccacggaggataccgccacatatttctgtgctaggggatggcctcttgcttattgggggcaagggacacaagtgtctgtttccgaagccaaaacaacgcccccttcagtctatccgctggcaccgggaagcgcggcacaaaccaatagtatggtaacgctcggatgtctcgtcaaggggtattttcccgagcctgtgacagtgacatggaattctgggagtcttagcagcggagtacatacttttccggcagtacttcaatccgatttgtacacgctctcctctagtgttacagttccaagctctccacgacctagtgagaccgttacatgtaacgtcgcgcatccggcctcttccactaaagtggataaaaagattgtgcccagggactgcggcggagggggctctggcggcggaggatctgggggagggggcagccaaattgtgttgacccagtccccggccataatgtccgcttctcctggcgagaaggttactatgacttgctcagcctcctccagtgtgtattatatgtactggtatcaacaaaagccgggctcttccccccggctccttatatacgacacgagtaatctggcaagtggcgtgcctgttagattttctgggtccggctctggaacttcatactccctgacaattagccgaatggaagccgaggacgcggcgacatactactgccagcaatggtcatcctatccgcctatcacttttggagtagggaccaaattggagttgaagcgggctgatgcggctcccacagttagtattttccctccgtccagtgaacaacttacctccgggggagcctccgttgtttgctttctgaacaacttttacccgaaagatataaatgtcaagtggaagatcgacggctcagagcgccaaaacggggtactcaactcatggacagatcaggatagtaaagattcaacttacagtatgtctagtaccctgacactgacgaaagatgaatacgaaagacataatagttatacctgtgaagctacacataagacttcaacctctcctattgtaaaatcattcaaccgaaacgaatgt

SEQ ID NO: 67 - DNA, 1338 bp, Artificial Sequence
  VL-linker-VH of DNP scFv (1BAF) coding sequence
caaattgtgttgacccagtccccggccataatgtccgcttctcctggcgagaaggttactatgacttgctcagcctcctccagtgtgtattatatgtactggtatcaacaaaagccgggctcttccccccggctccttatatacgacacgagtaatctggcaagtggcgtgcctgttagattttctgggtccggctctggaacttcatactccctgacaattagccgaatggaagccgaggacgcggcgacatactactgccagcaatggtcatcctatccgcctatcacttttggagtagggaccaaattggagttgaagcgggctgatgcggctcccacagttagtattttccctccgtccagtgaacaacttacctccgggggagcctccgttgtttgctttctgaacaacttttacccgaaagatataaatgtcaagtggaagatcgacggctcagagcgccaaaacggggtactcaactcatggacagatcaggatagtaaagattcaacttacagtatgtctagtaccctgacactgacgaaagatgaatacgaaagacataatagttatacctgtgaagctacacataagacttcaacctctcctattgtaaaatcattcaaccgaaacgaatgtggcggagggggctctggcggcggaggatctgggggagggggcagcgacgtacaactgcaagaatcaggtccgggactggtcaaacctagccagtctcagtccttgacgtgcaccgtgacgggctatagtataacgagtgattacgcttggaattggattcggcagtttccaggcaataagcttgagtggatgggctatatgtcttactccggctcaactaggtataatccgagccttcggtctcggatttctatcacaagagatacgagtaagaatcagttttttcttcaactcaaaagcgtcaccacggaggataccgccacatatttctgtgctaggggatggcctcttgcttattgggggcaagggacacaagtgtctgtttccgaagccaaaacaacgcccccttcagtctatccgctggcaccgggaagcgcggcacaaaccaatagtatggtaacgctcggatgtctcgtcaaggggtattttcccgagcctgtgacagtgacatggaattctgggagtcttagcagcggagtacatacttttccggcagtacttcaatccgatttgtacacgctctcctctagtgttacagttccaagctctccacgacctagtgagaccgttacatgtaacgtcgcgcatccggcctcttccactaaagtggataaaaagattgtgcccagggactgc

SEQ ID NO: 68 - DNA, 738 bp, Artificial Sequence
  VH-linker-VL of DNP scFv (XC) coding sequence
cagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtctcctcaggcggagggggctctggcggcggaggatctgggggagggggcagcgcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttc

SEQ ID NO: 69 - DNA, 762 bp, Artificial Sequence
  VL-linker-VH of DNP scFv (XC) coding sequence
gcccaagtgctgacccagactccatcgcctgtgtctgcagctgtgggaggcacagtcaccatcagttgccagtccagtgagagtgtttatggtaacagccgcttagcctggtatcagcagaaaccagggcagtctcccaagctcctgatctattatgcatccactctggcatctggggtcccttcgcggttcaaaggcagtggatctgggacacagttcactctcaccattagcgacctggagtgtgacgatgctgcctcttactactgtcaaggcggttattatagtggtaatcttgatgcgcttgctttcggcggagggaccgaggtggtggtcagaggtggcggagggggctctggcggcggaggatctgggggagggggcagccagcagctggagcagtccggaggaggagccgaaggaggcctggtcaagcctgggggatccctggaactctgctgcaaagcctctggattctccctcagtagtagctactgcatatgttgggtccgccaggctccagggaaggggctggagtggatcggatgcatttatgctggtagtagtggtagcacttactacgcgagctgggtgaatggccgattcactctctccagagacattgaccagagcacaggttgcctacaactgaacagtctgacagccgcggacacggccatgtattactgtgcgagagccccctatagtagtggctgggtcctctactttaacttgtggggcccaggcaccctggtcattgtc

SEQ ID NO: 70 - DNA, 789 bp, Artificial Sequence; identical to SEQ ID NO: 52
  VH-linker-VL of DNP scFv (Ab-3) coding sequence

SEQ ID NO: 71 - DNA, 789 bp, Artificial Sequence; identical to SEQ ID NO: 50
  DNP scFv VH-linker-VL of DNP scFv (Ab-3)

SEQ ID NO: 72 - protein, 15 aa, Artificial Sequence
  Glycine-serine linker
GGGGSGGGGSGGGGS

SEQ ID NO: 73 - protein, 20 aa, Artificial Sequence
  Glycine-serine linker
GGGGSGGGGSGGGGSGGGGS

SEQ ID NO: 74 - protein, 10 aa, Artificial Sequence
  Glycine-serine linker
GGGGSGGGGS

SEQ ID NO: 75 - protein, 5 aa, Artificial Sequence
  Glycine-serine linker
GGGGS

SEQ ID NO: 76 - DNA, 45 bp, Artificial Sequence
  Coding sequence of (G_4S)_3 linker
ggcggaggcggctccggaggaggagggtctggaggcgggggaagt

SEQ ID NO: 77 - DNA, 60 bp, Artificial Sequence
  Coding sequence of (G_4S)_4 linker
ggcgggggaggctccgggggaggcggctccggcggcgggggaagtggcgggggaggatcc

SEQ ID NO: 78 - protein, 12 aa, Artificial Sequence
  Short Spacer (IgG4 (short hinge))
ESKYGPPCPPCP

SEQ ID NO: 79 - DNA, 36 bp, Artificial Sequence
  Short Spacer (IgG4 (short hinge)) Coding Sequence
gagtctaagtacggtccaccttgccccccctgccct

SEQ ID NO: 80 - protein, 11 aa, Artificial Sequence
  Short Spacer 2, Alternative IgG4 linker
ESKYGPPCPPC

SEQ ID NO: 81 - protein, 119 aa, Artificial Sequence
  Medium Spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 229 aa, Artificial Sequence
  Long spacer with single (L235D) mutation
ESKYGPPCPPCPAPEFDGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 229 aa, Artificial Sequence
  Long spacer with double (L235D, N297Q) mutations
ESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - skipped

SEQ ID NO: 85 - protein, 22 aa, Artificial Sequence
  P2A
GSGATNFSLLKQAGDVEENPGP

SEQ ID NO: 86 - DNA, 66 bp, Artificial Sequence
  P2A Coding Sequence
ggaagtggtgcaaccaacttctctcttttgaaacaggctggagatgtcgaggagaaccctgggcct

SEQ ID NO: 87 - protein, 21 aa, Artificial Sequence
  T2A
GGGEGRGSLLTCGDVEENPGP

SEQ ID NO: 88 - DNA, 63 bp, Artificial Sequence
  T2A Coding Sequence
ggcggcggcgaagggcgaggaagtttgcttacctgcggggatgtggaggaaaaccccggtcct

SEQ ID NO: 89 - protein, 24 aa, Artificial Sequence
  E2A
GSGQCTNYALLKLAGDVESNPGPP

SEQ ID NO: 90 - protein, 25 aa, Artificial Sequence
  F2A
GSGVKQTLNFDLLKLAGDVESNPGP

SEQ ID NO: 91 - protein, 187 aa, Artificial Sequence
  Dihydrofolate reductase double mutant
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 92 - DNA, 561 bp, Artificial Sequence
  DHFRdm Coding Sequence
atggtaggttccctcaattgtatagtggctgtcagtcaaaacatgggtataggcaaaaatggcgacttcccgtggccgccgctcaggaacgaatcaagatacttccaacgcatgacgacaactagctctgtcgaagggaagcagaatctcgtcatcatgggtaagaagacgtggttctctataccagagaaaaatcgcccgttgaaagggaggataaacctcgtgctttcacgcgaattgaaggaacctccgcaaggggcccactttctgagtcgctcactcgacgatgcgttgaaactgactgaacagcctgagttggcaaacaaagtcgacatggtgtggatcgtgggaggaagcagtgtttataaagaagcaatgaaccaccccggacacctgaagcttttcgttacccgaataatgcaggactttgaatcagatacattcttccctgaaattgaccttgaaaaatataaacttctccctgaataccctggcgtgctgtccgacgtccaagaggaaaaaggtattaagtacaagtttgaggtatatgaaaaaaatgat

SEQ ID NO: 93 - protein, 323 aa, Artificial Sequence
  Truncated CD19
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 94 - DNA, 969 bp, Artificial Sequence
  CD19t Coding Sequence
atgcccccacctcgcctgctcttttttctcttgttcctcacgcccatggaagtccggcccgaggagccacttgtagtcaaggtagaagagggagacaacgcagtgctgcagtgccttaaaggcacaagtgatgggcccacccaacaactgacatggagtcgcgagtcacctcttaaacccttcctcaaattgagtctcggcttgccgggcttgggcattcatatgcgacctctggctatatggctgttcatttttaatgtttcccagcagatggggggtttttatttgtgccagccaggcccgccaagcgaaaaagcctggcagccaggatggacagtcaatgtagaaggatctggtgagctttttaggtggaacgtatctgatttggggggacttggatgcggtttgaagaaccgatcttccgagggtccgagctctcctagtgggaaactgatgtctcctaagctgtacgtttgggcgaaagaccggcccgagatttgggagggcgagccgccctgtgtgccgcctcgagactcccttaatcagtccctttcccaggacctgacgatggcacctgggagtacgctgtggcttagttgtggggtgccgcctgatagtgtctcccggggtcccctttcctggacacacgtgcatccaaaaggcccgaagtctcttttgtctttggagcttaaagacgatcgccctgctcgcgatatgtgggtgatggaaacgggattgcttctgccacgcgccaccgcccaggatgcggggaagtattactgccatcgagggaacctgacaatgagctttcatctggaaatcactgccagaccagtactgtggcactggcttttgcggacgggcggctggaaagtgagcgcagtgacactggcgtatctcattttctgcctttgtagtctcgtaggtattttgcacttgcaacgcgcgcttgtgcttcgcagaaagcgg

SEQ ID NO: 95 - protein, 135 aa, Artificial Sequence
  truncated Her2 (Her2)
MLLLVTSLLLCELPHPAFLLIPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILI

SEQ ID NO: 96 - protein, 120 aa, Artificial Sequence
  Her2tG
CHPECQPQNGSVTCFGPEADQCVACAHYKDPPFCVARCPSGVKPDLSYMPIWKFPDEEGACQPCPINCTHSCVDLDDKGCPAEQRASPLTGGGSGGGSIISAVVGILLVVVLGVVFGILI

SEQ ID NO: 97 - protein, 335 aa, Artificial Sequence
  EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 98 - protein, 22 aa, Artificial Sequence
  GM-CSF Signal Peptide
MLLLVTSLLLCELPHPAFLLIP

SEQ ID NO: 99 - protein, 20 aa, Artificial Sequence
  CD8 signal peptide
MALPVTALLLPLALLLHAAR

SEQ ID NO: 100 - protein, 6 aa, Artificial Sequence
  His tag
HHHHHH

SEQ ID NO: 101 - protein, 7 aa, Artificial Sequence
  Flag tag
DYKDDDD

SEQ ID NO: 102 - protein, 8 aa, Artificial Sequence
  Flag tag
DYKDDDDK

SEQ ID NO: 103 - protein, 7 aa, Artificial Sequence
  Flag tag
DYKDDDK

SEQ ID NO: 104 - protein, 8 aa, Artificial Sequence
  Xpress tag
DLYDDDDK

SEQ ID NO: 105 - protein, 15 aa, Artificial Sequence
  Avi tag
GLNDIFEAQKIEWHE

SEQ ID NO: 106 - protein, 26 aa, Artificial Sequence
  Calmodulin binding peptide (CBP) tag
KRRWKKNFIAVSAANRFKKISSSGAL

SEQ ID NO: 107 - protein, 6 aa, Artificial Sequence
  Polyglutamate tag
EEEEEE

SEQ ID NO: 108 - protein, 9 aa, Artificial Sequence
  HA tag
YPYDVPDYA

SEQ ID NO: 109 - protein, 9 aa, Artificial Sequence
  HA tag
YAYDVPDYA

SEQ ID NO: 110 - protein, 9 aa, Artificial Sequence
  HA tag
YDVPDYASL

SEQ ID NO: 111 - protein, 10 aa, Artificial Sequence
  Myc tag
EQKLISEEDL

SEQ ID NO: 112 - protein, 8 aa, Artificial Sequence
  Strep tag
WRHPQFGG

SEQ ID NO: 113 - protein, 8 aa, Artificial Sequence
  STREP(R) tag II
WSHPQFEK

SEQ ID NO: 114 - protein, 13 aa, Artificial Sequence
  Softag 1
SLAELLNAGLGGS

SEQ ID NO: 115 - protein, 8 aa, Artificial Sequence
  Softag 3
TQDPSRVG

SEQ ID NO: 116 - protein, 14 aa, Artificial Sequence
  V5 tag
GKPIPNPLLGLDST

SEQ ID NO: 117 - DNA, 399 bp, Artificial Sequence
  MND promoter
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 118-119 - skipped (2 entries)

SEQ ID NO: 120 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 121 - protein, 112 aa, Artificial Sequence
  CD3-zeta ICD
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122-123 - skipped (2 entries)

SEQ ID NO: 124 - DNA, 336 bp, Artificial Sequence
  CD3-zeta ICD Coding Sequence
agagtgaagttcagcagatccgccgacgctcctgcctatcagcagggacagaaccagctgtacaacgagctgaacctggggagaagagaagagtacgacgtgctggataagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctcgt

SEQ ID NO: 125 - skipped

SEQ ID NO: 126 - protein, 69 aa, Artificial Sequence
  CD8-alpha Hinge/Transmembrane domain
TTTPAPRPPTPAPTIASQPLSLRPEACRPAAGGAVHTRGLDFACDIYIWAPLAGTCGVLLLSLVITLYC

SEQ ID NO: 127 - protein, 75 aa, Artificial Sequence
  Alternative CD8 hinge/Transmembrane domain
ESKYGPAKPTTTPAPRPPTPAPTIASQPLSLRPEACRPAAGGAVHTRGLDFACDIYIWAPLAGTCGVLLLSLVIT

SEQ ID NO: 128 - protein, 21 aa, Artificial Sequence
  Alternative CD8 hinge/Transmembrane domain (residues 183-203 of UniProt ID P01732-1)
IYIWAPLAGTCGVLLLSLVIT

SEQ ID NO: 129 - protein, 48 aa, Artificial Sequence
  Alternative Human CD8 alpha chain hinge (residues 135-182 of UniProt ID P01732-1)
AKPTTTPAPRPPTPAPTIASQPLSLRPEACRPAAGGAVHTRGLDFACD

SEQ ID NO: 130 - protein, 154 aa, Artificial Sequence
  4-1BB/CD3z intracellular signaling domain
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - DNA, 462 bp, Artificial Sequence
  Coding sequence of 4-1BB/CD3z intracellular signaling domains
aaaaggggacgcaaaaaactcctttacattttcaaacagccctttatgcgacctgtacaaacaactcaggaagaggatggctgcagctgtaggtttcccgaggaagaagagggtggctgcgagctcagagtgaagttcagcagatccgccgacgctcctgcctatcagcagggacagaaccagctgtacaacgagctgaacctggggagaagagaagagtacgacgtgctggataagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgcactgcacatgcaggccctgccacctcgt

SEQ ID NO: 132 - DNA, 462 bp, Artificial Sequence
  Alternative Coding sequence of 4-1BB/CD3z intracellular signaling domains
aaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 133 - DNA, 942 bp, Artificial Sequence; identical to SEQ ID NO: 15
  EBD (L384M, M421G, G521R) Pairs with estrogen analog CMP8 Coding Sequence

SEQ ID NO: 134 - DNA, 942 bp, Artificial Sequence
  G400V/M543/L544A ER(T2) Coding Sequence
tctgctggcgatatgagagccgccaacctgtggcccagccccctgatgatcaagcggagcaagaagaacagcctggccctgagcctgaccgccgatcagatggtgtccgctctgctggacgccgagccccctatcctgtacagcgagtacgaccccaccagacccttcagcgaggccagcatgatgggcctgctgaccaacctggccgaccgggagctggtgcacatgatcaactgggccaagcgggtgcccggcttcgtggacctgaccctgcacgaccaggtccacctgctggaatgtgcctggctggaaatcctgatgatcggcctcgtgtggagaagcatggaacaccccgtgaagctgctgttcgcccccaacctgctcctggaccggaaccagggaaagtgcgtggagggcatggtggagatcttcgacatgctgctggccacctccagccggttccggatgatgaacctgcagggcgaggaattcgtgtgcctgaagtccatcatcctgctgaacagcggcgtgtacaccttcctgtcatccaccctgaagtccctggaagagaaggaccacatccaccgggtgctggacaagatcaccgacaccctgatccacctgatggccaaggctggcctgacactccagcagcagcaccagagactggcccagctgctgctgatcctgagccacatccggcacatgagcaacaagggaatggaacacctgtacagcatgaagtgcaagaacgtggtgcccctgtacgacctgctgctcgaggctgccgatgcccacagactgcacgcccctacaagcagaggcggagccagcgtggaggaaaccgaccagtctcacctggccaccgccggcagcacaagcagccacagcctgcagaagtactacatcaccggcgaggccgagggattccctgccaccgtg

SEQ ID NO: 135 - protein, 55 aa, Artificial Sequence
  CD3-epsilon ICD
KNRKAKAKPVTRGAGAGGRQRGQNKERPPPVPNPDYEPIRKGQRDLYSGLNQRRI

SEQ ID NO: 136 - protein, 770 aa, Artificial Sequence
  CA-STAT3: Mutations at A662C and N664C for constitutive activation
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDCTCILLSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 137 - DNA, 2310 bp, Artificial Sequence
  CA-STAT3: Mutations at A662C and N664C for constitutive activation Coding Sequence
atggctcagtggaaccagctgcaacagctggacaccagatacctcgagcagctccaccagctgtactctgacagcttcccaatggaactgaggcagtttctggctccctggattgagagccaggattgggcctatgccgcctccaaagaatctcacgccacactcgtgttccacaacctgctgggagagatcgaccagcagtacagccggttcctgcaagagtccaacgtgctgtaccagcacaacctgagaagaatcaagcagttcctgcagagcagatacctggaaaagcccatggaaatcgccagaattgtggcccggtgtctgtgggaagaaagcagactgctgcaaacagccgccactgctgcacagcaaggcggacaggctaatcatcctacagccgccgtggtcaccgagaaacaacagatgctggaacagcatctgcaggacgtgcggaagagagtgcaggacctggaacagaaaatgaaggtggtggaaaacctgcaggatgacttcgacttcaactacaagaccctgaaaagccagggcgacatgcaggatctgaatggcaacaaccagtccgtgaccaggcagaaaatgcagcagctcgagcagatgctcacagccctggatcagatgcggagatccattgtgtctgagctggccggactgctgtccgccatggaatacgtgcagaaaactctgaccgatgaggaactggctgactggaagcggagacagcagattgcctgtatcggcggacctcctaacatctgcctggaccggctggaaaactggatcacctctctggccgaatctcagctgcagaccagacagcaaatcaagaagctggaagaactccagcaaaaggtgtcctacaagggcgaccccatcgtgcagcatagacctatgctcgaggaacggatcgtggaactgttcagaaacctgatgaagtccgccttcgtggtggaacggcagccttgtatgcctatgcaccccgatagacccctggtcatcaagaccggcgtgcagtttaccaccaaagtccggctgctggtcaagttccccgagctgaactaccagctgaaaatcaaagtgtgcatcgacaaggactccggcgacgtggcagctctgagaggcagcagaaagttcaatatcctgggcaccaacaccaaagtgatgaacatggaagagagcaacaacggctccctgagcgccgagtttaaacacctgacactgagagaacagagatgcggcaacggcggcagagccaattgtgatgccagcctgatcgtgacagaggaactgcacctgatcaccttcgagacagaggtgtaccatcagggcctgaagattgacctggaaacccactctctgcccgtggtcgtgatcagcaacatctgtcagatgcccaacgcctgggccagcatcctgtggtacaacatgctgacaaacaaccccaagaacgtcaacttcttcaccaagcctccaatcggcacctgggatcaagtggccgaagtgctgtcttggcagttcagcagcaccaccaagaggggcctgagcattgagcagctgaccacactggcagagaaactgcttggccctggcgtgaactactccggctgtcagatcacatgggccaagttctgtaaagaaaacatggccggcaagggcttcagcttctgggtctggctggataacatcatcgacctcgtcaagaagtacattctcgccctgtggaacgagggctatatcatgggcttcatctctaaagagagagagcgggccatcctgagcacaaagcctccaggcacttttctgctgaggttctccgagtccagcaaagaaggcggcgtcacctttacctgggtcgagaaggatatcagcggcaagacccagatccagagcgtggaaccttacacaaaacagcagctgaacaacatgagcttcgccgagatcatcatggggtacaagatcatggactgtacctgcatcctgctgagccctctggtgtatctgtaccctgacatccccaaagaggaagccttcggcaagtactgcagacccgagtctcaagagcaccccgaagccgatcctggatctgccgctccttacctgaaaactaagttcatctgcgtgacccctaccacctgtagcaacaccatcgatctgcctatgagccccagaacactggactccctgatgcagttcggaaacaatggcgagggcgcagaacctagtgccggcggacaatttgagtccctgaccttcgatatggaactcaccagcgagtgcgccacctctcctatg

SEQ ID NO: 138 - protein, 794 aa, Artificial Sequence
  caSTAT5a: Mutations at H298R and S710F for constitutive activation
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAERLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNAFADAGGSSATYMDQAPSPAVCPQAPYNMYPQNPDHVLDQDGEFDLDETMDVARHVEELLRRPMDSLDSRLSPPAGLFTSARGSLS

SEQ ID NO: 139 - protein, 45 aa, Artificial Sequence
  CD3-delta ICD
GHETGRLSGAADTQALLRNDQVYQPLRDRDDAQYSHLGGNWARNK

SEQ ID NO: 140 - DNA, 2382 bp, Artificial Sequence
  caSTAT5a: Mutations at H298R and S710F for constitutive activation Coding Sequence
atggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcgcctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgattccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatttgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctca

SEQ ID NO: 141 - protein, 787 aa, Artificial Sequence
  caSTAB5b: Mutation at N642H for constitutive activation
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWHLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 142 - protein, 2361 aa, Artificial Sequence
  caSTAB5b: Mutation at N642H for constitutive activation Coding Sequence
ATGGCTGTGTGGATACAAGCTCAGCAGCTCCAAGGAGAAGCCCTTCATCAGATGCAAGCGTTATATGGCCAGCATTTTCCCATTGAGGTGCGGCATTATTTATCCCAGTGGATTGAAAGCCAAGCATGGGACTCAGTAGATCTTGATAATCCACAGGAGAACATTAAGGCCACCCAGCTCCTGGAGGGCCTGGTGCAGGAGCTGCAGAAGAAGGCAGAGCACCAGGTGGGGGAAGATGGGTTTTTACTGAAGATCAAGCTGGGGCACTATGCCACACAGCTCCAGAACACGTATGACCGCTGCCCCATGGAGCTGGTCCGCTGCATCCGCCATATATTGTACAATGAACAGAGGTTGGTCCGAGAAGCCAACAATGGTAGCTCTCCAGCTGGAAGCCTTGCTGATGCCATGTCCCAGAAACACCTCCAGATCAACCAGACGTTTGAGGAGCTGCGACTGGTCACGCAGGACACAGAGAATGAGTTAAAAAAGCTGCAGCAGACTCAGGAGTACTTCATCATCCAGTACCAGGAGAGCCTGAGGATCCAAGCTCAGTTTGGCCCGCTGGCCCAGCTGAGCCCCCAGGAGCGTCTGAGCCGGGAGACGGCCCTCCAGCAGAAGCAAGTGTCTCTGGAGGCCTGGTTGCAGCGTGAGGCACAGACACTGCAGCAGTACCGCGTGGAGCTGGCCGAGAAGCACCAGAAGACCCTGCAGCTGCTGCGGAAGCAGCAGACCATCATCCTGGATGACGAGCTGATCCAGTGGAAGCGGCGGCAGCAGCTGGCCGGGAACGGCGGGCCCCCCGAGGGCAGCCTGGACGTGCTACAGTCCTGGTGTGAGAAGTTGGCCGAGATCATCTGGCAGAACCGGCAGCAGATCCGCAGGGCTGAGCACCTCTGCCAGCAGCTGCCCATCCCCGGCCCAGTGGAGGAGATGCTGGCCGAGGTCAACGCCACCATCACGGACATTATCTCAGCCCTGGTGACCAGCACGTTCATCATTGAGAAGCAGCCTCCTCAGGTCCTGAAGACCCAGACCAAGTTTGCAGCCACTGTGCGCCTGCTGGTGGGCGGGAAGCTGAACGTGCACATGAACCCCCCCCAGGTGAAGGCCACCATCATCAGTGAGCAGCAGGCCAAGTCTCTGCTCAAGAACGAGAACACCCGCAATGATTACAGTGGCGAGATCTTGAACAACTGCTGCGTCATGGAGTACCACCAAGCCACAGGCACCCTTAGTGCCCACTTCAGGAATATGTCCCTGAAACGAATTAAGAGGTCAGACCGTCGTGGGGCAGAGTCGGTGACAGAAGAAAAATTTACAATCCTGTTTGAATCCCAGTTCAGTGTTGGTGGAAATGAGCTGGTTTTTCAAGTCAAGACCCTGTCCCTGCCAGTGGTGGTGATCGTTCATGGCAGCCAGGACAACAATGCGACGGCCACTGTTCTCTGGGACAATGCTTTTGCAGAGCCTGGCAGGGTGCCATTTGCCGTGCCTGACAAAGTGCTGTGGCCACAGCTGTGTGAGGCGCTCAACATGAAATTCAAGGCCGAAGTGCAGAGCAACCGGGGCCTGACCAAGGAGAACCTCGTGTTCCTGGCGCAGAAACTGTTCAACAACAGCAGCAGCCACCTGGAGGACTACAGTGGCCTGTCTGTGTCCTGGTCCCAGTTCAACAGGGAGAATTTACCAGGACGGAATTACACTTTCTGGCAATGGTTTGACGGTGTGATGGAAGTGTTAAAAAAACATCTCAAGCCTCATTGGAATGATGGGGCCATTTTGGGGTTTGTAAACAAGCAACAGGCCCATGACCTACTCATTAACAAGCCAGATGGGACCTTCCTCCTGAGATTCAGTGACTCAGAAATTGGCGGCATCACCATTGCTTGGAAGTTTGATTCTCAGGAAAGAATGTTTTGGCACCTGATGCCTTTTACCACCAGAGACTTCTCCATTCGGTCCCTAGCCGACCGCTTGGGAGACTTGAATTACCTTATCTACGTGTTTCCTGATCGGCCAAAAGATGAAGTATACTCCAAATACTACACACCAGTTCCCTGCGAGTCTGCTACTGCTAAAGCTGTTGATGGATACGTGAAGCCACAGATCAAGCAAGTGGTCCCTGAGTTTGTGAACGCATCTGCAGATGCCGGGGGCGGCAGCGCCACGTACATGGACCAGGCCCCCTCCCCAGCTGTGTGTCCCCAGGCTCACTATAACATGTACCCACAGAACCCTGACTCAGTCCTTGACACCGATGGGGACTTCGATCTGGAGGACACAATGGACGTAGCGCGGCGTGTGGAGGAGCTCCTGGGCCGGCCAATGGACAGTCAGTGGATCCCGCACGCACAATCG

SEQ ID NO: 143 - protein, 113 aa, Artificial Sequence
  CD3-zeta ICD
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 144 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 145 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 146 - protein, 384 aa, Homo sapiens
MPQLDSGGGGAGGGDDLGAPDELLAFQDEGEEQDDKSRDSAAGPERDLAELKSSLVNESEGAAGGAGIPGVPGAGAGARGEAEALGREHAAQRLFPDKLPEPLEDGLKAPECTSGMYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTETNWPRELKDGNGQESLSMSSSSSPA

SEQ ID NO: 147 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 148 - protein, 5 aa, Artificial Sequence
  (Gly4Ser)n linker sequence
  MISC_FEATURE (1)..(5): Can be repeated n times, wherin n is an integer of 1, 2, 3, 4, 5, 6, 7, 8, 9 or 10
GGGGS

SEQ ID NO: 149 - protein, 42 aa, Artificial Sequence
  OX40 ICD
ALYLLRRDQRLPPDAHKPPGGGSFRTPIQEEQADAHSTLAKI

SEQ ID NO: 150 - protein, 4 aa, Artificial Sequence
  (Gly3Ser)n linker sequence
  MISC_FEATURE (1)..(4): Can be repeated n times, wherin n is an integer of 1, 2, 3, 4, 5, 6, 7, 8, 9 or 10
GGGS

SEQ ID NO: 151 - protein, 4 aa, Artificial Sequence
  (GGGG)n linker sequence
  MISC_FEATURE (1)..(4): Can be repeated n times, wherin n is an integer of 1, 2, 3, 4, 5, 6, 7, 8, 9 or 10
GGGG

SEQ ID NO: 152 - protein, 12 aa, Artificial Sequence
  (GSAGSAAGSGEF)n linker sequence
  MISC_FEATURE (1)..(12): Can be repeated n times, wherin n is an integer of 1, 2, 3, 4, 5, 6, 7, 8, 9 or 10
GSAGSAAGSGEF

SEQ ID NO: 153 - protein, 48 aa, Artificial Sequence
  CD27 ICD
QRRKYRSNKGESPVEPAEPCHYSCPREEEGSTIPIQEDYRKPEPACSP

SEQ ID NO: 154 - protein, 9 aa, Artificial Sequence
  (Gly3Ser)n(Gly4Ser)n linker sequence
  MISC_FEATURE (1)..(4): Can be repeated n times, wherin n is an integer of 1, 2, 3, 4, 5, 6, 7, 8, 9 or 10
  MISC_FEATURE (5)..(9): Can be repeated n times, wherin n is an integer of 1, 2, 3, 4, 5, 6, 7, 8, 9 or 10
GGGSGGGGS

SEQ ID NO: 155 - protein, 7 aa, Artificial Sequence
  (Gly3Ser)n(Gly2Ser)n linker sequence
  MISC_FEATURE (1)..(4): Can be repeated n times, wherin n is an integer of 1, 2, 3, 4, 5, 6, 7, 8, 9 or 10
  MISC_FEATURE (5)..(7): Can be repeated n times, wherin n is an integer of 1, 2, 3, 4, 5, 6, 7, 8, 9 or 10
GGGSGGS

SEQ ID NO: 156 - protein, 9 aa, Artificial Sequence
  (Gly3Ser)n(Gly4Ser)1 linker sequence
  MISC_FEATURE (1)..(4): Can be repeated n times, wherin n is an integer of 1, 2, 3, 4, 5, 6, 7, 8, 9 or 10
GGGSGGGGS

SEQ ID NO: 157 - skipped

SEQ ID NO: 158 - protein, 20 aa, Artificial Sequence
  (Gly4Ser)4 linker sequence
GGGGSGGGGSGGGGSGGGGS

SEQ ID NO: 159 - protein, 15 aa, Artificial Sequence
  (Gly4Ser)3 linker sequence
GGGGSGGGGSGGGGS

SEQ ID NO: 160 - protein, 10 aa, Artificial Sequence
  (Gly4Ser)2 linker sequence
GGGGSGGGGS

SEQ ID NO: 161 - skipped

SEQ ID NO: 162 - protein, 5 aa, Artificial Sequence
  (Gly4Ser)1 linker sequence
GGGGS

SEQ ID NO: 163 - protein, 8 aa, Artificial Sequence
  (Gly3Ser)2 linker sequence
GGGSGGGS

SEQ ID NO: 164 - protein, 4 aa, Artificial Sequence
  (Gly3Ser)1 linker sequence
GGGS

SEQ ID NO: 165 - skipped

SEQ ID NO: 166 - protein, 6 aa, Artificial Sequence
  (Gly2Ser)2 linker sequence
GGSGGS

SEQ ID NO: 167 - protein, 11 aa, Artificial Sequence
  GGSGGGSGGSG linker sequence
GGSGGGSGGSG

SEQ ID NO: 168 - protein, 10 aa, Artificial Sequence
  GGSGGGSGSG linker sequence
GGSGGGSGSG

SEQ ID NO: 169 - skipped

SEQ ID NO: 170 - protein, 8 aa, Artificial Sequence
  GGSGGGSG linker sequence
GGSGGGSG

SEQ ID NO: 171-179 - skipped (9 entries)

SEQ ID NO: 180 - protein, 41 aa, Artificial Sequence
  CD28 ICD (residues 180-220 of UniProt ID P10747)
RSKRSRLLHSDYMNMTPRRPGPTRKHYQPYAPPRDFAAYRS

SEQ ID NO: 181 - protein, 41 aa, Artificial Sequence
  CD28 ICD
RSKRSRGGHSDYMNMTPRRPGPTRKHYQPYAPPRDFAAYRS

SEQ ID NO: 182 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 183 - protein, 4 aa, Artificial Sequence
  Degron sequence
RRRG

SEQ ID NO: 184 - protein, 5 aa, Artificial Sequence
  Degron sequence #2
RRRGN

(19) United States
(12) Patent Application Publication
(10) Pub. No.: US 2025/0074959 A1
(43) Pub. Date: Mar. 6, 2025

(54) ACTIVITY-INDUCIBLE FUSION PROTEINS HAVING A TRANSCRIPTION FACTOR AND A HEAT SHOCK PROTEIN 90 BINDING DOMAIN

(71) Applicant: Seattle Children's Hospital d/b/a Seattle Children's Research Institute, Seattle, WA (US)

(72) Inventors: Michael C. Jensen, Seattle, WA (US); Jia Wei, Seattle, WA (US); Taylor Kimiko-Ha Ishida, Seattle, WA (US)

(73) Assignee: Seattle Children's Hospital d/b/a Seattle Children's Research Institute, Seattle, WA (US)

(21) Appl. No.: 18/560,903

(22) PCT Filed: May 13, 2022
(86) PCT No.: PCT/US22/29307

§ 371 Date: Nov. 14, 2023

Related U.S. Application Data
(60) Provisional application No. 63/189,000, filed on May 14, 2021.

(51) Int. Cl. C07K 14/47; A61K 39/00; C07K 14/72; C12N 5/0783

(52) U.S. Cl. CPC C07K 14/4705; A61K 40/11; A61K 40/30; C07K 14/721; C12N 5/0636; C07K 2319/02; C07K 2319/03; C12N 2510/00

(57) ABSTRACT

Activity-inducible fusion proteins including a transcription factor and a heat-shock protein 90 (hsp90) binding domain are described. The activity of the transcription factor is regulated utilizing a drug molecule that binds the hsp90 binding domain. In the absence of the drug molecule, the transcription factor is in an inactive state but can be activated in the presence of the drug molecule. The activity-inducible transcription factors can be used to alter the activation state of immune cells, and optionally can be co-expressed with a chimeric antigen receptor (CAR).

### CROSS-REFERENCE TO RELATED APPLICATIONS

This application is a U.S. National Phase based on International Patent Application No. PCT/US2022/029307, filed on May 13, 2022, which claims priority to U.S. Provisional Patent Application No. 63/189,000 filed May 14, 2021, both of which are incorporated herein by reference in their entirety as if fully set forth herein.

### REFERENCE TO SEQUENCE LISTING

The Sequence Listing associated with this application is provided in text format in lieu of a paper copy and is hereby incorporated by reference into the specification. The name of the text file containing the Sequence Listing is 44656629_ST25.txt. The text file is 278,848 bytes, was created on Nov. 14, 2023, and is being submitted electronically via Patent Center.

### FIELD OF THE DISCLOSURE

The current disclosure provides activity-inducible fusion proteins having a transcription factor and a heat-shock protein 90 (hsp90) binding domain. The activity of the transcription factor is regulated utilizing a drug molecule that binds the hsp90 binding domain. In the absence of the drug molecule, the transcription factor fusion proteins is in an inactive state but can be activated in the presence of the drug molecule. The activity-inducible transcription factor fusion proteins can be used to alter immune cell activity, and optionally can be co-expressed with a chimeric antigen receptor (CAR).

### BACKGROUND OF THE DISCLOSURE

Using genetic engineering, significant progress has been made in activating and directing cells of the immune system to kill cancer cells and infected cells. For example, T cells have been genetically engineered to express molecules having extracellular components that bind particular target antigens and intracellular components that direct actions of the T cell when the extracellular component has bound the target antigen. As an example, the extracellular component can be designed to bind target antigens found on cancer cells or infected cells and, when bound, the intracellular component activates the T cell to destroy the bound cell. Examples of such molecules include chimeric antigen receptors (CAR).

Clinical trials with CAR-expressing T cells (CAR-T) have shown positive responses and cancer reduction in some patients. However, in others they have failed to provide prolonged anti-cancer activity. One reason for a lack of prolonged success may be that the T cells do not receive strong or diverse enough activation signals when bound to a targeted cell.

Upregulated expression of transcription factors in immune cells can increase immune cell function and lead to more effective and prolonged therapeutic effects. However, if left unregulated, negative results may occur. For example, significant toxicities can arise due to engraftment-induced cytokine storm (cytokine release syndrome), tumor lysis syndromes (TLS) and ongoing B cell cytopenias, each of which are attributable to unregulated functional outputs of activated CAR-expressing immune cells. Such toxicities can limit the applicability of CAR-based therapies.

Methods to eliminate CAR-T cells such as suicide gene-mediated elimination of these cells, for example, can ameliorate such toxicities; however, this approach risks premature attenuation of anti-tumor activity and can significantly impact curative potential. As such, there is a need to identify methods for controlling the activity of immune cells, such as gene-modified immune cells.

### SUMMARY OF THE DISCLOSURE

The current disclosure provides fusion proteins whose activation state can be controlled through the administration of drug molecules. The fusion proteins include a transcription factor and a heat shock protein 90 (hsp90) binding domain that binds the drug molecule. In the absence of the drug molecule, the hsp90 binding domain is bound by hsp90 preventing the transcription factor from dimerizing with other transcription factors required for the initiation of gene transcription. When the drug molecule is present, the drug molecule can displace the bound hsp90 from the hsp90 binding domain site and/or otherwise results in a conformational change, such that the transcription factors can initiate gene transcription. This ability to control gene expression and immune cell activation states in vivo provides an important improvement in cellular immunotherapies, including for example, CAR-based cellular immunotherapies.

The current disclosure achieves these advances by incorporating a hsp90 binding domain and a transcription factor within a fusion protein. Certain embodiments disclosed herein utilize a hormone binding domain as the hsp90 binding domain. The hormone binding domain can be an estrogen receptor binding domain (EBD). The EBD can be derived from the natural estrogen receptor but include at least one mutation such that the EBD no longer binds estrogen, but instead binds a drug molecule with a higher affinity than hsp90. Exemplary drug molecules include tamoxifen or derivatives or metabolites thereof with fewer side effects such as 4-hydroxytamoxifen (4-OHT), CMP8, or ES8.

In the absence of tamoxifen or a derivative or metabolite thereof, hsp90 binds the EBD and the transcription factor fusion protein is in the “OFF” state. Nanomolar concentrations of cytosolic tamoxifen, however, can actively out compete hsp90 for EBD binding and/or otherwise result in a conformational change, allowing, for example, transcription factor dimerization and the initiation of gene transcription.

One benefit of the current disclosure is the ability to control the activity of a fusion protein without reliance on protein stabilization/destabilization, for example through the incorporation of a degron sequence

Certain examples of the disclosure utilize constitutively activated signal transducer and activator of transcription proteins (caSTATs) as the transcription factor within the fusion proteins. caSTATs activate gene transcription that results in proliferation and activation of immune cells. Other exemplary transcription factors that can be used include transcription factor 7 (TCF7), c-Myc, B-cell lymphoma protein (BCL6), Activator protein 1 (AP-1), Nuclear factor kB (NF-kb), Forkhead box (FOX) and the Sp/KLF family of transcription factors.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIGS. 1A-1D. (1A) A diagram showing how an estrogen analog activates caSTAT-ER fusion proteins, an exemplary transcription factor of the disclosure. (1B-1D) show the different combinations of fusion proteins to independently control different transcription factors with different estrogen analogs. (1B) Diagram showing how CMP8 activates caSTAT5-EBD fusion protein. (1C) Diagram showing how 4-OHT activates caSTAT3-ER fusion protein. (1D) Diagram showing CMP8 activating caSTAT5-EBD fusion protein and 4-OHT activating caSTAT3-ER fusion protein.

FIG. 2. Dual-Glo Luciferase Assay of caSTAT5a and STAT5a transduced 293/STAT5-RE-Luc2P. Human CA-STAT5a was made by site-directed mutagenesis (H298R & S710F).

FIG. 3. Dual-Glo luciferase assay of different versions of caSTAT5aER in 293t/STAT5RE-Luc2P cells. caSTAT5a was fused with the mutated Estrogen Receptor Ligand Binding domain, ER (T2), in various ways (N-terminal or C-terminal, with or without linkers). ER (T2) contains three mutations, G400V/M543A/L544A, which makes it sensitive to 4-hydroxy tamoxifen (4-OHT) and insensitive to 17 β-oestradiol. The STAT5 reporter cells 293t/STAT5-RE-Luc2P were seeded in a 96-well plate. Plasmid DNA of STAT5aER (T2) variants were transfected into these cells in combination with pRL-SV40 at 10:1 molar ratios using Lipofectamine 2000. The pRL-SV40 was a Renilla luciferase plasmid control that was used to normalize the transfection variation. Then the cells were treated with four doses of 4-OHT. 24-h later, Dual-Glo luciferase assay was performed according to the protocol from Promega.

FIG. 4. CA-STAT5aER was activated upon 4-OHT treatment in the transduced H9 cells. The anti-STAT5 694Y antibody was used to detect phosphorylated caSTAT5 (activated form) band on the western blot.

FIG. 5. Growth curve of the transduced CD8 cells in RPMI media supplemented with or without IL2 and IL15. Cells were treated with 100 nM 4-OHT every 3-4 days and then changed to 500 nM 4-OHT on day 18.

FIG. 6. Sequences supporting the disclosure.

### DETAILED DESCRIPTION

Using genetic engineering, significant progress has been made in activating and directing cells of the immune system to kill cancer cells and infected cells. For example, T cells have been genetically engineered to express molecules having extracellular components that bind particular target antigens and intracellular components that direct actions of the T cell when the extracellular component has bound the target antigen. As an example, the extracellular component can be designed to bind target antigens found on cancer cells or infected cells and, when bound, the intracellular component activates the T cell to destroy the bound cell. Examples of such molecules include chimeric antigen receptors (CAR).

Clinical trials with CAR-expressing T cells (CAR-T) have shown positive responses and cancer reduction in some patients. However, in others they have failed to provide prolonged anti-cancer activity. One reason for a lack of prolonged success may be that the T cells do not receive strong or diverse enough activation signals when bound to a targeted cell.

Upregulated expression of transcription factors in modified immune cells can increase immune cell function and lead to more effective and prolonged therapeutic effects. However, if left unregulated, negative results may occur. For example, significant toxicities can arise due to engraftment-induced cytokine storm (cytokine release syndrome), tumor lysis syndromes (TLS) and ongoing B cell cytopenias, each of which are attributable to unregulated functional outputs of activated CAR-expressing immune cells. Such toxicities can limit the applicability of immune cell-based therapies.

Methods to eliminate CAR-T cells such as suicide gene-mediated elimination of these cells, for example, can ameliorate such toxicities; however, this approach risks premature attenuation of anti-tumor activity and can significantly impact curative potential. Additional methods to control the activity of proteins is through the use of degron sequences that can lead to the controllable degradation or stability of the protein. However, these effects are delayed, relying on cellular degradation of proteins. As such, there is a need to identify more rapid and controllable methods for controlling the activity of gene-modified immune cells.

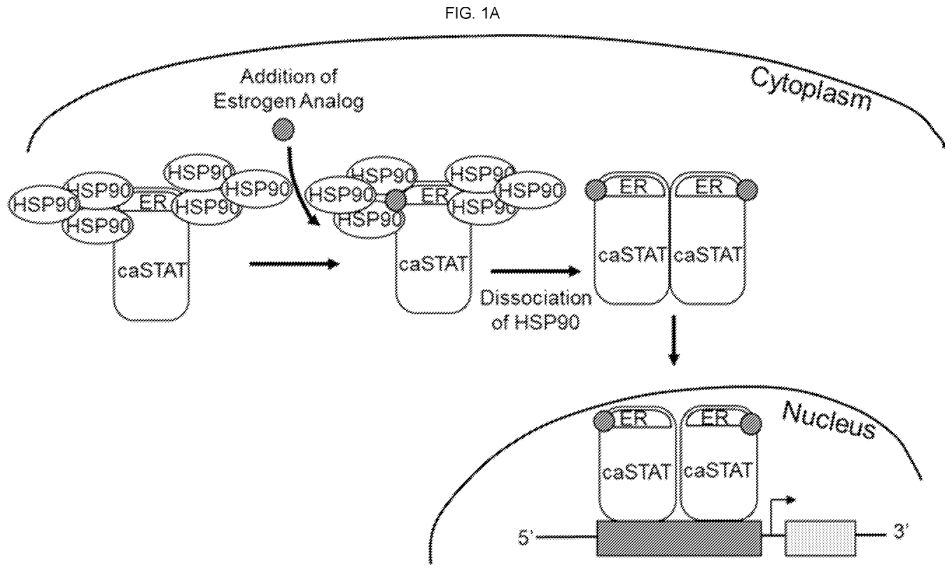

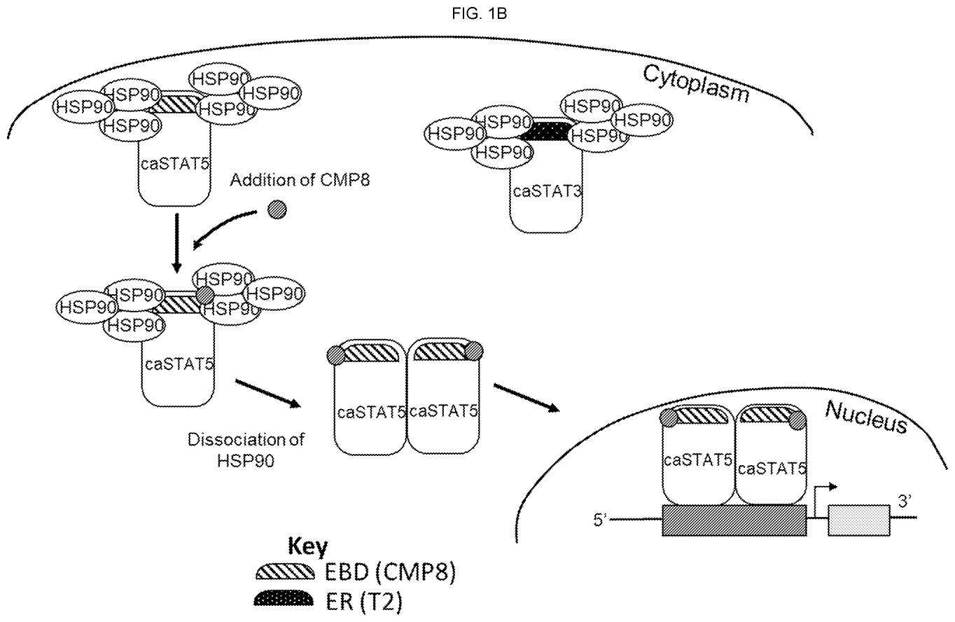

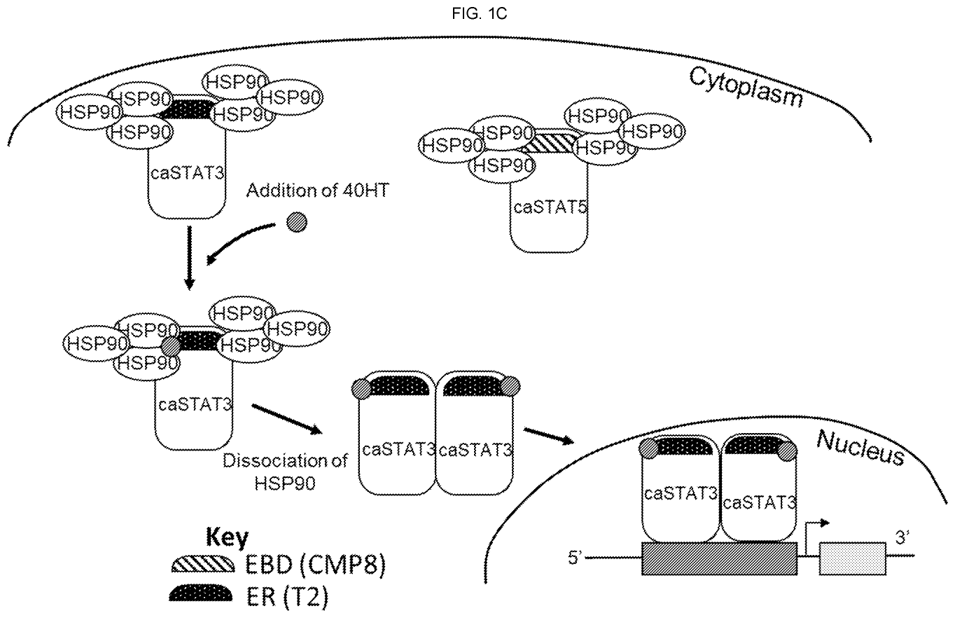

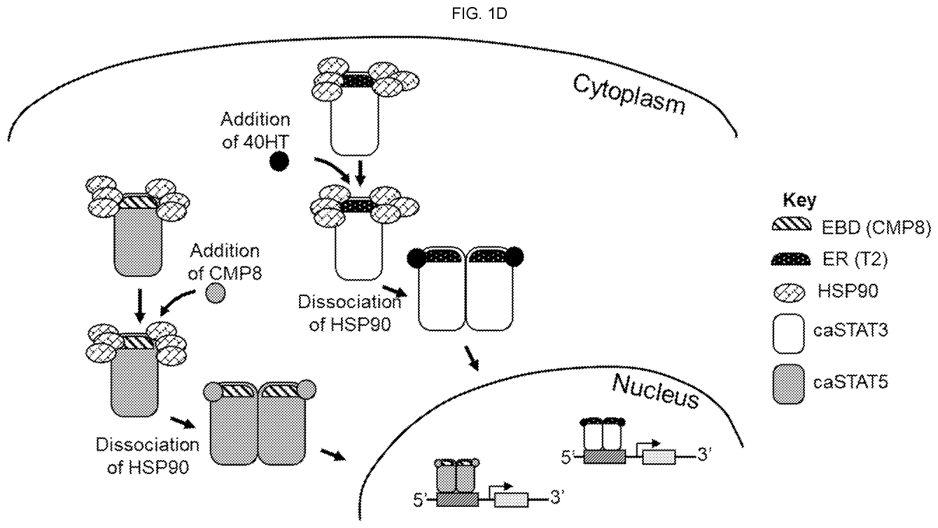

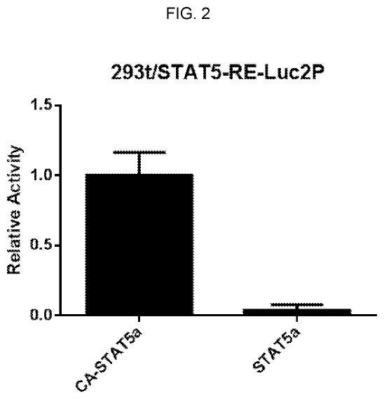

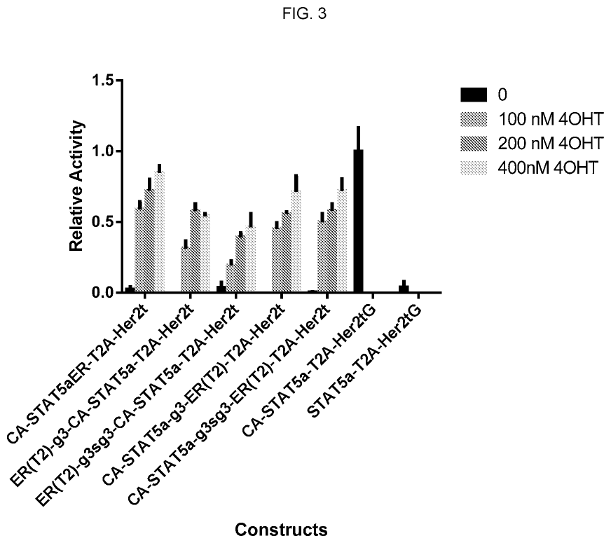

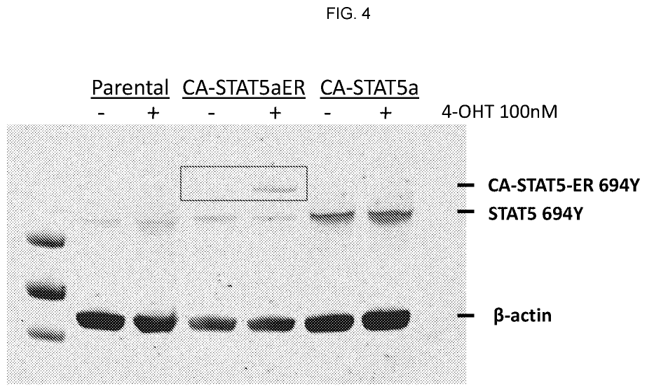

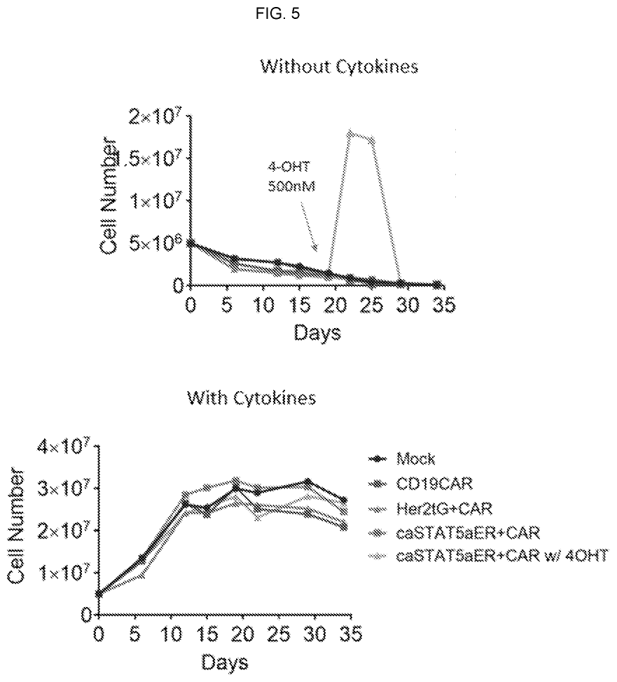

The current disclosure provides fusion proteins whose activation state can be controlled through the administration of drug molecules. The fusion proteins include a transcription factor and a heat shock protein 90 (hsp90) binding domain that binds the drug molecule. Referring to the exemplary embodiment depicted in FIG. 1, the ability of the transcription factor to dimerize and initiate gene transcription is controlled with the administration of the drug molecule. In the absence of the drug molecule, the hsp90 binding domain is bound by hsp90 preventing the transcription factor from dimerizing with other transcription factors required for the initiation of gene transcription. When the drug molecule is present, the drug molecule can displace the bound hsp90 from the hsp90 binding domain site and/or otherwise result in a conformational change, such that the transcription factors can dimerize, leading to the initiation of gene transcription. This ability to control gene expression in vivo provides an important improvement to cellular immunotherapies, such as CAR-based cellular immunotherapies.

One benefit of the current disclosure is the ability to control the activity of a fusion protein without reliance on protein stabilization/destabilization, for example through the incorporation of a degron sequence. As used herein, a degron sequence refers to an amino acid sequence recombinantly linked to a fusion protein for the purpose of controlling the stability/degradation of the protein. Degron sequences are typically linked to fusion proteins at the C-terminal end. Examples are described in, for example, US 2014/0255361, and include RRRG (SEQ ID NO: 183) and RRRGN (SEQ ID NO: 184).

Certain examples of the disclosure utilize constitutively activated signal transducer and activator of transcription proteins (caSTATs) as the transcription factor within the fusion proteins. caSTATs activate gene transcription that results in proliferation and activation of immune cells. Other exemplary transcription factors that can be used include transcription factor 7 (TCF7), c-Myc, B-cell lymphoma protein (BCL6), Activator protein 1 (AP-1), Nuclear factor kB (NF-kb), Forkhead box (FOX), and the Sp/KLF family of transcription factors.

Certain embodiments disclosed herein utilize a hormone binding domain as the hsp90 binding domain. The hormone binding domain can be an estrogen receptor binding domain (EBD). The EBD can be derived from the natural estrogen receptor but include at least one mutation such that the EBD no longer binds estrogen, but instead binds a drug molecule with a higher affinity than hsp90. Exemplary drug molecules include tamoxifen or derivatives or metabolites thereof with fewer side effects such as 4-hydroxytamoxifen (4-OHT), CMP8 or ES8.

In the absence of tamoxifen or a derivative or metabolite thereof, hsp90 binds the EBD and the transcription factor fusion protein is in the “OFF” state. Nanomolar concentrations of cytosolic tamoxifen, however, actively out compete hsp90 for EBD binding, allowing transcription factor dimerization and the initiation of gene transcription.

Hsp90 binding domains can also be derived from the binding domains for cortisol, androgens, progesterone, and aldosterone. Further, hsp90 binding domains can be derived from numerous other proteins that bind hsp90, commonly referred to as hsp90 clients. Hsp90 clients typically include hormone receptors, transcription factors, and kinases, among other types of molecules.

Aspects of the current disclosure are now described with additional detail and options as follows: (i) Transcription Factors; (ii) Drug Molecules and hsp90 Binding Domains; (iii) Fusion Proteins; (iv) Chimeric Antigen Receptors (iv-a) Ligand Binding Domains; (iv-b) Intracellular Signaling Domains; (iv-c) Transmembrane Domains; (iv-d) Linkers; (iv-e) Tags and Selectable Markers; (v) Cells Genetically Modified to Express Activity-Inducible Fusion Proteins; (vi) Methods to Modify Cells Ex Vivo and In Vivo; (vii) Production of Activity-Inducible Fusion Proteins; (viii) Modified Formulations, Modifying Formulations, and Drug Compositions; (ix) Methods of Use; (x) Kits; (xi) hsp90 Clients; (xii) Variants; (xiii) Exemplary Embodiments; (xiv) Experimental Examples; and (xv) Closing Paragraphs. These headings are provided for organizational purposes only and do not limit the scope or interpretation of the disclosure.

(i) Transcription Factors. Intracellular pathways for immune cell (e.g., T-cell) activation signals mainly include PLC-γ activation pathways, Ras-MAP kinase activation pathways, Jak-STAT pathways, and PI3K pathways. The Jak-STAT pathway is a common pathway for cytokine signaling and the PI3K pathway is part of the TCR and co-stimulatory receptor signaling pathways. After a series of cascades of signal transduction molecules, transcription factors are eventually activated and enter the nucleus to regulate the transcription of related target genes.

Signal Transducer of Activation and Transcription (STAT) proteins can include constitutively active STAT proteins (ca-STATs), meaning that the STAT is continuously expressed, independent of the presence of endogenous regulators. Several STAT proteins are known. For instance, IL-4 activates STAT6 and, indirectly, STAT5 (Lischke, et al., J Biol Chem 273, 31222-31229, 1998; Rolling et al., FEBS Lett 393, 53-56, 1996) while IL-2 activates STAT3 and STAT5 (reviewed in (Leonard and O'Shea, Annu Rev Immunol 16, 293-322, 1998)). STAT3 and STAT6 molecules are involved in B cell development and differentiation. STAT3 results in the expression of c-Fos, HIF-1α, c-Myc, Sox2, Zeb1, Bcl-2, Mcl-1, and Bcl-xL and is implicated in plasma cell differentiation (Reljic et al., J Exp Med 192, 1841-1848, 2000). STAT6 affects the choice of immunoglobulin isotype (IgE) during class switch recombination (Kaplan et al., Immunity 4, 313-319, 1996; Shimoda et al., Nature 380, 630-633, 1996). There are two known forms of STAT5, STAT5a and STAT5b, which are encoded by two different, tandemly-linked genes. They play both unique and redundant roles in the response of cells to a wide variety of growth factors (Teglund et al., Cell 93, 841-850, 1998). STAT5a results in the expression of Bcl2, Junb, Id2, NDRG1, DNAJC6, CBS, PPP2R2B, ST3GAL1, SAMD4A, SSH2, and MAP3K5 while STAT5b results in the expression of DOCK8, SNX9, LNPEP, SKAP1, PTGER1, and FOXP3. CA-STAT5a increases T cell survival and proliferation, enhances cytotoxic effects of CD8 T cells and their tumor retention. In particular examples, CA-STAT5a and CA-STAT5b activity can be induced with a small molecule drug when immune cell proliferation would be beneficial.

Transcription factor TCF7 includes a DNA binding domain, which binds one or more DNA consensus motifs, and an alpha-helix (HMG box). This gene is expressed predominantly in T-cells and plays a critical role in natural killer cell and innate lymphoid cell development. The encoded protein forms a complex with beta-catenin and activates transcription through a Wnt/β-catenin signaling pathway.

The c-Myc protein is a transcription factor that has been shown to both upregulate and downregulate a variety of target genes. Heterodimerization with its protein partner, Max, is required for sequence-specific DNA binding to a specific E box element as well as for biological activity (Oster et al., Adv. Cancer Res., 84:81-154, 2002). Transactivation of target gene promoters by c-Myc also requires binding of factors to the amino-terminal transactivation domain, such as TRRAP and Tip48/49 (Oster et al., Adv. Cancer Res., 84:81-154, 2002). c-Myc upregulates several proliferative genes, such as cyclin D, cyclin E, and cdk4 (Oster et al., Adv. Cancer Res., 84:81-154, 2002). Also, antiproliferative genes, such as cyclin-dependent kinase inhibitors (CDK-I) p21^{Cip1}, p15^{Ink4b}, p27^{Kip1}, and several of the gadd genes, can be repressed by c-Myc to facilitate cell cycle progression (Oster et al., Adv. Cancer Res., 84:81-154, 2002). The tumor suppressor, p19^{ARF} (ARF), which is induced by c-Myc, Ras and E2F, mediates p53 activation by sequestering Mdm2 and thus inhibiting the Mdm2-dependent degradation of p53 (Bates et al., Nature, 395:124-125, 1998; Pomerantz et al., Cell, 92:713-723, 1998; Zhang et al., Cell, 92:725-734, 1998; Zindy et al., Genes Dev., 12:2424-2433, 1998).

B-cell lymphoma protein (BCL6) is an evolutionarily conserved zinc finger transcription factor which contains an N-terminal POZ/BTB domain. BCL6 acts as a sequence-specific repressor of transcription and has been shown to modulate the STAT-dependent Interleukin 4 (IL-4) responses of B cells. It interacts with several corepressor complexes to inhibit transcription.

AP-1 (Activator protein 1) is a transcriptional activator in the cell and is a heterodimer composed of c-Fos and c-Jun. It responds to various stimuli by regulating gene expression, including cytokines, growth factors, stress, bacterial and viral infections; therefore AP-1 controls many cellular processes, including differentiation, proliferation and apoptosis. AP-1 up-regulates the transcription of a gene containing TPA DNA response element (TRE; 5′-TGAG/CTCA-3′). The AP-1 heterodimer is formed by a leucine zipper and initiates the expression of a gene by binding a specific conserved sequence to the gene.

c-Jun, a cellular homolog of the v-Jun oncogene, is a member of the BZip protein family. It is a major component of the AP-1 transcriptional complex, which recognizes AP-1 and CRE-like sites in gene promoters. c-Jun is a protein, required for the progression through the Gi phase of the cell cycle.

NF-kB (Nuclear factor kB) generally exists as a homo- or heterodimer. In resting cells, NF-kB dimers are dispersed in the cytoplasm by binding to their inhibitory protein IkB through a non-covalent bond. Many factors, including endoplasmic reticulum stress, can activate NF-kB. Upon activation, NF-kB enters the nucleus, binds to specific proteins on the DNA module, induces the production of specific mRNAs, and finally can transcribe, produce and release various cytokines.

FOX (Forkhead box) proteins are a family of transcription factors that play important roles in regulating the expression of genes involved in cell growth, proliferation, differentiation, and longevity. The defining feature of FOX proteins is the forkhead box, a sequence of 80 to 100 amino acids forming a motif that binds to DNA. This forkhead motif is also known as the winged helix due to the butterfly-like appearance of the loops in the protein structure of the domain. Forkhead genes are a subgroup of the helix-turn-helix class of proteins. Many other genes encoding FOX proteins have been identified. For example, the FOXF2 gene encodes forkhead box F2, one of many human homologues of the Drosophila melanogaster transcription factor forkhead. Some FOX genes are downstream targets of the hedgehog signaling pathway. Members of the class O regulate metabolism, cellular proliferation, stress tolerance and possibly lifespan.

The Sp/KLF family (specificity protein/Kruppel-like factor) is a family of transcription factors, including the Kruppel-like factors as well as Sp1, Sp2, Sp3, Sp4, Sp8, Sp9; and possibly Sp5 and Sp7. KLF14 is also designated Sp6. The Kruppel-like family of transcription factors (Klfs), have been extensively studied for their roles in cell proliferation, differentiation and survival. All KLF family members are characterized by their three Cys2 His2 zinc fingers located at the C-terminus separated by a highly conserved H/C link. DNA binding studies demonstrated that the KLFs have similar affinities for different GC-rich sites, or sites with CACCC homology, and can compete with each other for the occupation of such sites. KLFs also share a high degree of homology between the specificity protein (Sp) family of zinc-finger transcription factors and bind similar, if not the same sites, in a large number of genes. The following human genes encode Kruppel-like factors: KLF1, KLF2, KLF3, KLF4, KLF5, KLF6, KLF7, KLF8, KLF9, KLF10, KLF11, KLF12, KLF13, KLF14, KLF15, KLF16, and KLF17.

Additional examples of transcription factors include NFAT1, NFAT2, NFAT3, NFAT4, NFAT5, and V-Myb Avian Myeloblastosis Viral Oncogene Homolog (c-Myb).

(ii) Drug Molecules and hsp90 Binding Domains. Drug molecules used with activity-inducible fusion proteins disclosed herein are able to out-compete hsp90 for binding to a hsp90 binding domain present on the activity-inducible fusion protein and/or otherwise result in a conformation change that permits dimerization. In certain examples, the hsp90 binding domain is a hormone binding domain or modified form thereof.

In some alternatives, the drug molecule is a small molecule estrogen analog. Small molecule estrogen analogs include tamoxifen and salts and metabolites thereof, as well as compounds with structural similarity as described herein.

Tamoxifen is an estrogen antagonist/partial agonist that is an FDA-approved and commercially available drug. Tamoxifen has a proven safety record, favorable pharmacokinetic profile, excellent tissue distribution and a low partition coefficient between the extracellular space and cytosol. Tamoxifen is frequently administered orally as a pharmaceutically acceptable salt. For example, Tamoxifen citrate (RN 54965-24-1, M.W. 563.643) is indicated for treatment of metastatic breast cancer, and as an adjuvant for the treatment of breast cancer in women following mastectomy axillary dissection, and breast irradiation. Tamoxifen citrate is also indicated to reduce incidence of breast cancer in women at high risk for breast cancer.

Tamoxifen, CAS RN: 10540-29-1, is also known as 2-(4-((1Z)-1,2-diphenyl-1-butenyl) phenoxy)-N,N-dimethyl-ethanamine, or (Z)-2-(para-(1,2-Diphenyl-1-butenyl) phenoxy)-N, N-dimethylamine (IUPAC), and has a molecular formula of C₂₆H₂₉NO and a molecular weight (M.W.) of 371.52 g/mol.

Metabolites of tamoxifen that can be useful in some approaches described herein, include the major metabolites N-desmethyltamoxifen (RN 31750-48-8, M.W. 357.494) and 4-hydroxytamoxifen (4-OHT) (RN 68392-35-8, M.W. 387.52, Afimoxifene). These compounds are well known and described in Robinson et al. (Metabolites, pharmacodynamics, and pharmacokinetics of tamoxifen in rats and mice compared to the breast cancer patient. Drug Metab Dispos January 1991 19:36-43). Additional metabolites, useful in some approaches described herein, include cis-4-hydroxytamoxifen (RN 174592, M.W. 387.52; Afimoxifene, E-isomer), and 4′-hydroxytamoxifen ((Z)-4-(1-(4-(2-(dimethylamino) ethoxy)phenyl)-1-phenylbut-1-en-2-yl) pheno-I) as described in Crewe et al. (Metabolism of Tamoxifen by recombinant human cytochrome P-450 enzymes: Formation of the 4-hydroxy, 4′-hydroxy and N-desmethyl metabolites and isomerization of trans-4-hydroxytamoxifen, Drug Metab Dispos, 30 (8): 869-874, 2002).

Compounds with structural similarity to tamoxifen, useful in some approaches described herein, include cis-tamoxifen (RN 13002-65-8, M.W. 371.521), 4-methyltamoxifen (RN 73717-95-5, M.W. 385.548), N-desmethyltamoxifen (RN 31750-48-8, M.W. 357.494), (Z)-desethyl methyl tamoxifen (RN 15917-50-7, M.W. 357.494), (E)-desethyl methyl tamoxifen (RN 31750-45-5, M.W. 357.494), trans-4-hydoxytamoxifen (RN 68047 Jun. 3, M.W. 387.52), Afimoxifene (RN 68392-35-8, M.W. 387.52, 4-hydroxytamoxifen), Afimoxifene, E-isomer (RN 174592-47-3, M.W. 387.52), 4-chlorotamoxifen (RN 77588-46-6, M.W. 405.966), 4-fluorotamoxifen (RN 73617-96-6, M.W. 389.511), Toremifene (RN 89778-26-7, M.W. 405.966), desethyl tamoxifen (RN 19957-51-8, M.W. 343.47), (E)-desethyl tamoxifen (RN 97151 Oct. 5, M.W. 343.47), (Z)-desethyl tamoxifen (RN 97151 Nov. 6, M.W. 343.47), Miproxifene (RN 129612-87-9, M.W. 429.6), 2-(p-(beta-ethyl-alpha-phenyl styryl) phenoxy)triethylamine (RN 749-86-0, M.W. 399.575), Droloxifene (RN 82413-20-5, M.W. 387.52), 4-iodo-tamoxifen (RN 116057-68-2, M.W. 497.413), dihydrotamoxifen (RN 109640-20-2, M.W. 373.537), (E)-N,N-dimethyl-2-(4-(1-(2-methylphenyl)-2-phenyl-1-butenyl) phenoxy) ethanamine (RN 97150-96-4, M.W. 385.548), 4-hydroxytoremifene (RN 110503-62-3, M.W. 421.965); and pharmaceutically acceptable salts, hydrates or solvates thereof.

Citrate salts of tamoxifen, or citrate salts of compounds with structural similarity to tamoxifen, useful in some approaches described herein, include tamoxifen citrate (RN 54965-24-1, M.W. 563.64), 2-(p-(1,2-diphenyl-1-butenyl) phenoxy)-N, N-dimethylethylamine citrate (RN 7244-97-5, 563.64), (E)-tamoxifen citrate (RN 76487-65-5, M.W. 563.64), Toremifene citrate (RN 89778-27-8, M.W. 598.088), Droloxifene citrate (RN 97752-20-0, M.W. 579.64), 2-(p-(1,2-bis(p-methoxyphenyl)-1-butenyl) phenoxy)triethylamine citrate (RN 42920-39-8, M.W. 651.748), 2-(4-(1,2-diphenylethenyl) phenoxy)-N, N-diethyl-ethanamine 2-hydroxy-1,2,3-propanetricarboxylate (RN 40297-42-5, M.W. 563.643), 2-(p-(alpha-phenyl styryl) phenoxy)triethylamine citrate (RN 102433-95-4, M.W. 563.64), 2-(p-(2-(p-methoxyphenyl)-1-phenyl-1-butenyl) phenoxy)triethylamine citrate (1:1) (RN 42824-34-0, M.W. 637.72), 2-(p-(1-(p-methoxyphenyl)-2-phenylpropenyl) phenoxy)triethylamine citrate (RN 13554-24-0, M.W. 607.696), 2-(p-(alpha-(p-methoxyphenyl) styryl) phenoxy)triethylamine citrate monohydrate (RN 13542-71-7, M.W. 593.669), 2-(p-(p-methoxy-alpha-phenylphenethyl) phenoxy)triethylamine citrate (RN 16421-72-0, M.W. 595.685), alpha-(p-(2-(diethylamino) ethoxy)phenyl)-beta-ethyl-p-methoxy-alpha-pheny-lphenethyl alcohol citrate (1:1) (RN 35263-93-5, M.W. 639.737), 1-(p-(2-(diethylamino) ethoxy)phenyl)-2-(p-methoxyphenyl)-1-phenylethanol citrate (M.W. 611.68), alpha-p-(2-(diethyl amino) ethoxy)phenyl)-beta-ethyl-alpha-(p-hydroxyphenyl)-p-methoxyphenethy-| alcohol citrate (RN 35263-96-8, M.W. 655.737), and 2-(p-(p-methoxy-alpha-methylphenethyl) phenoxy)-triethylamine citrate (RN 15624-34-7, M.W. 533.614).

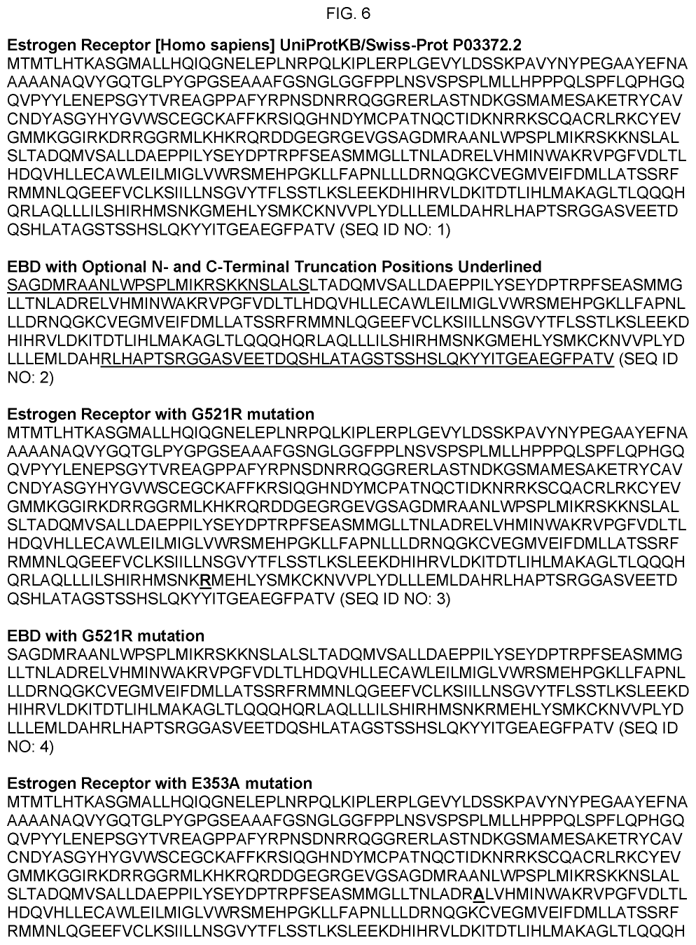

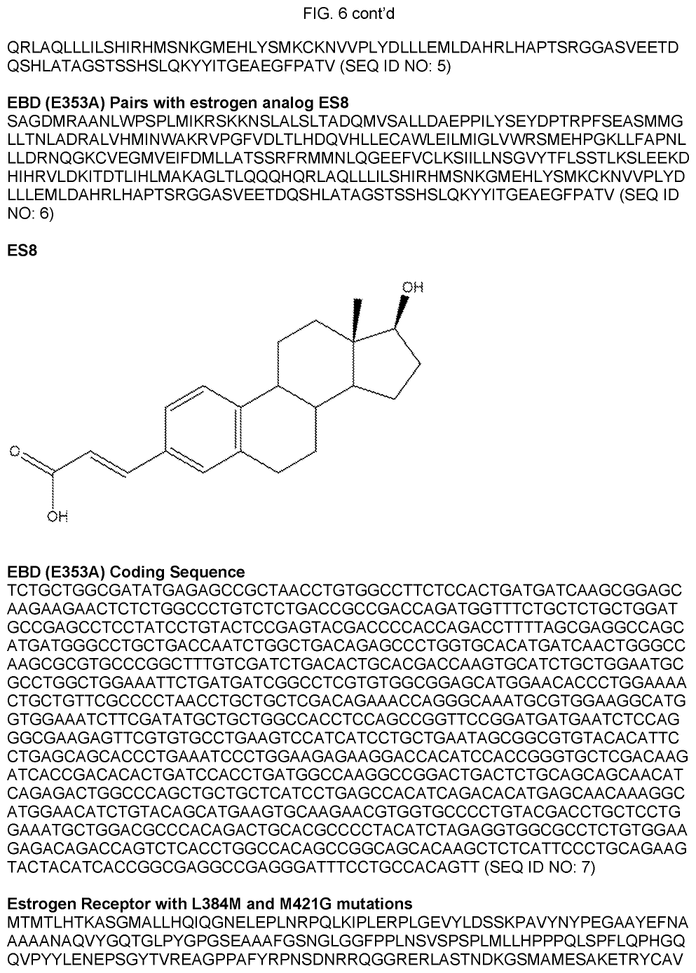

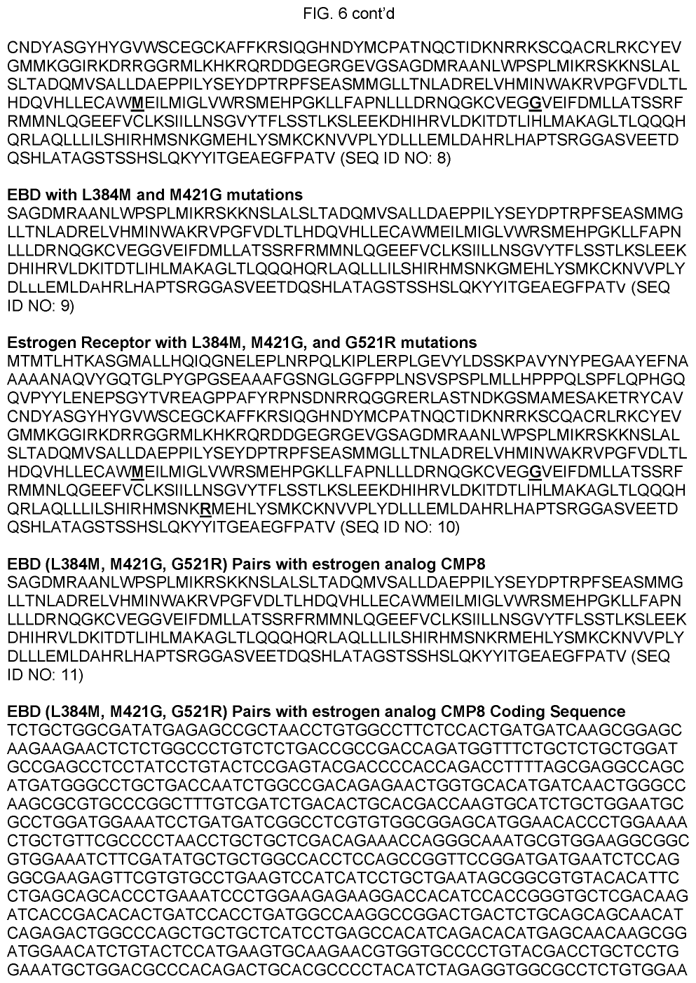

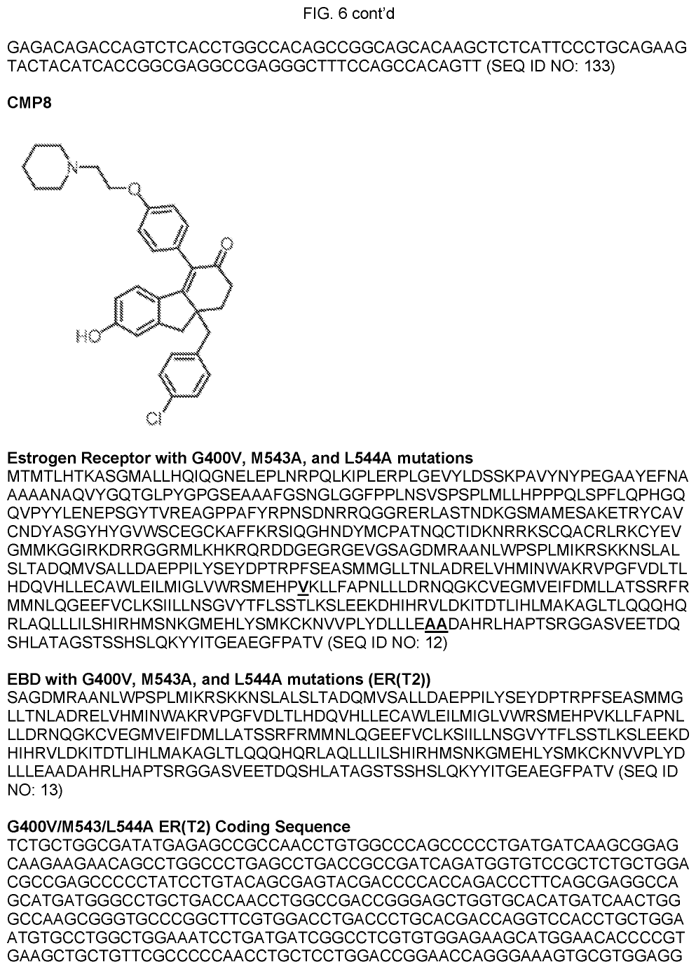

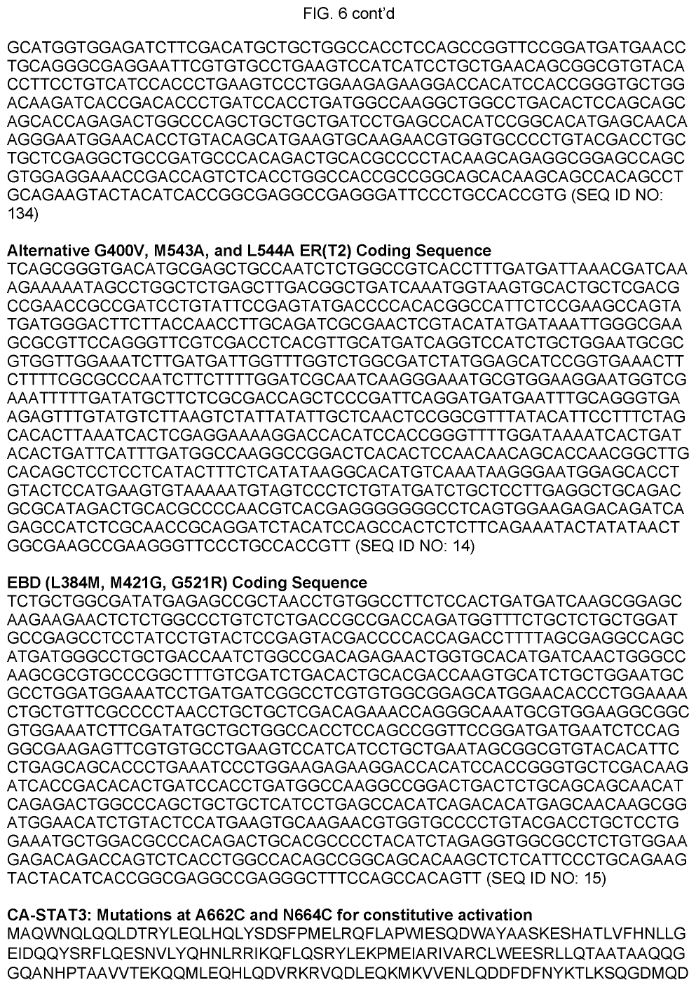

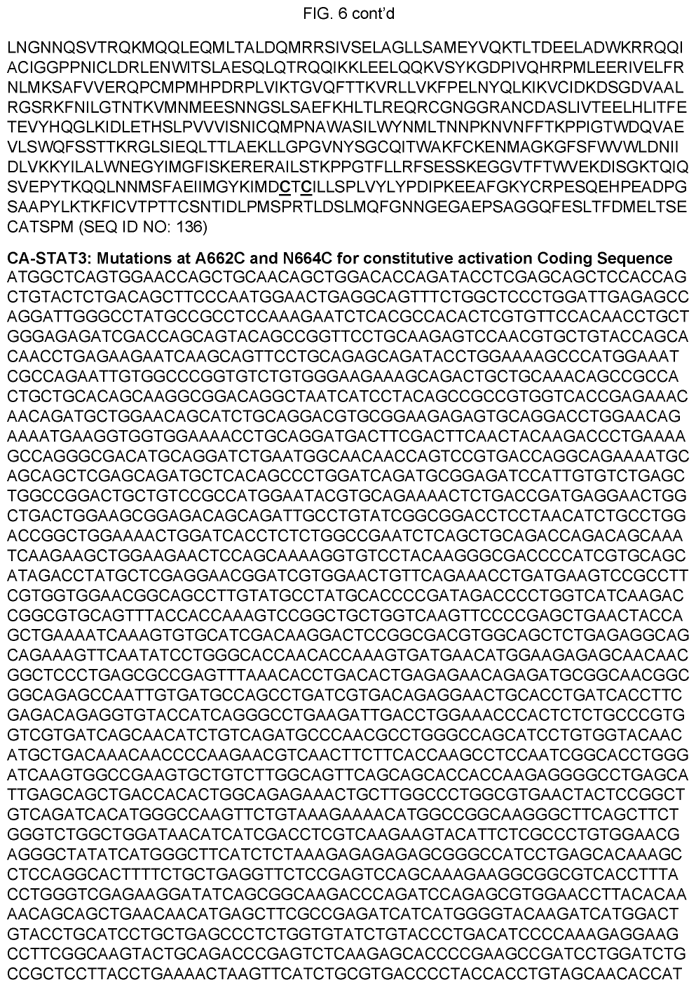

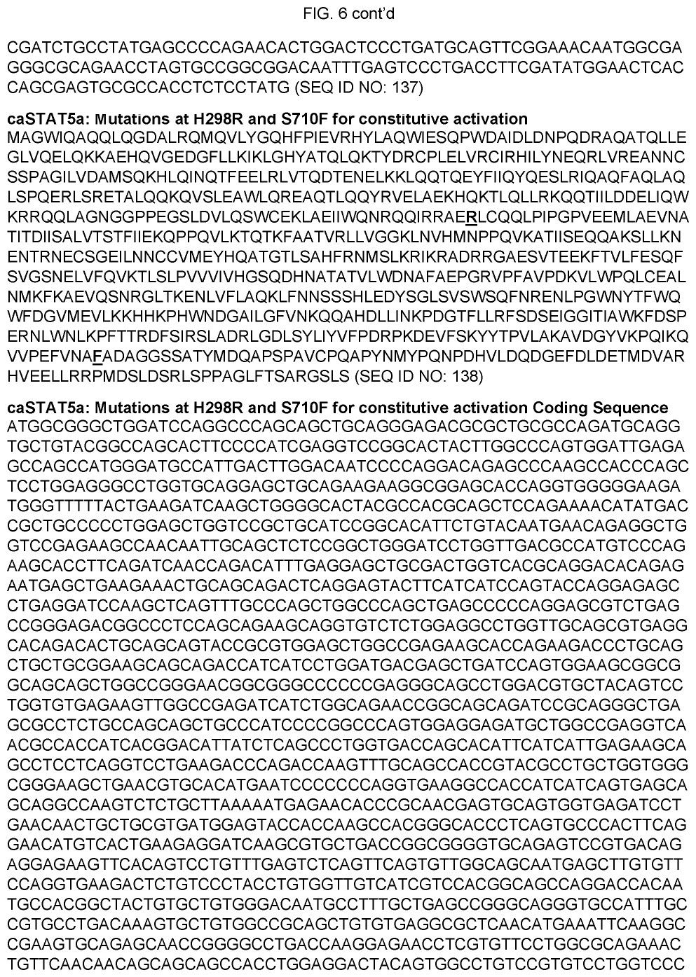

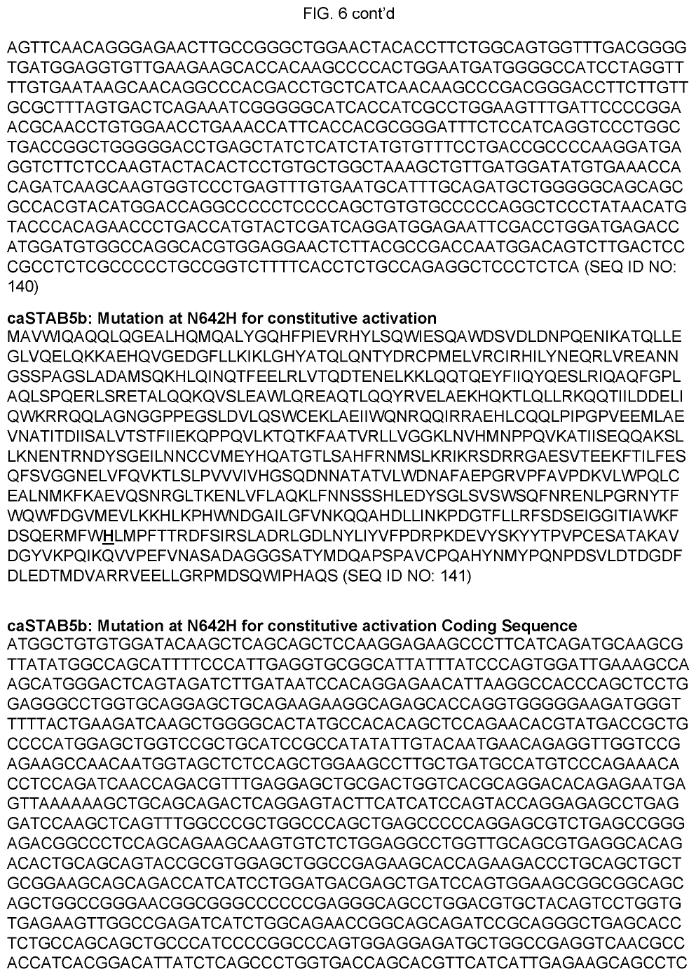

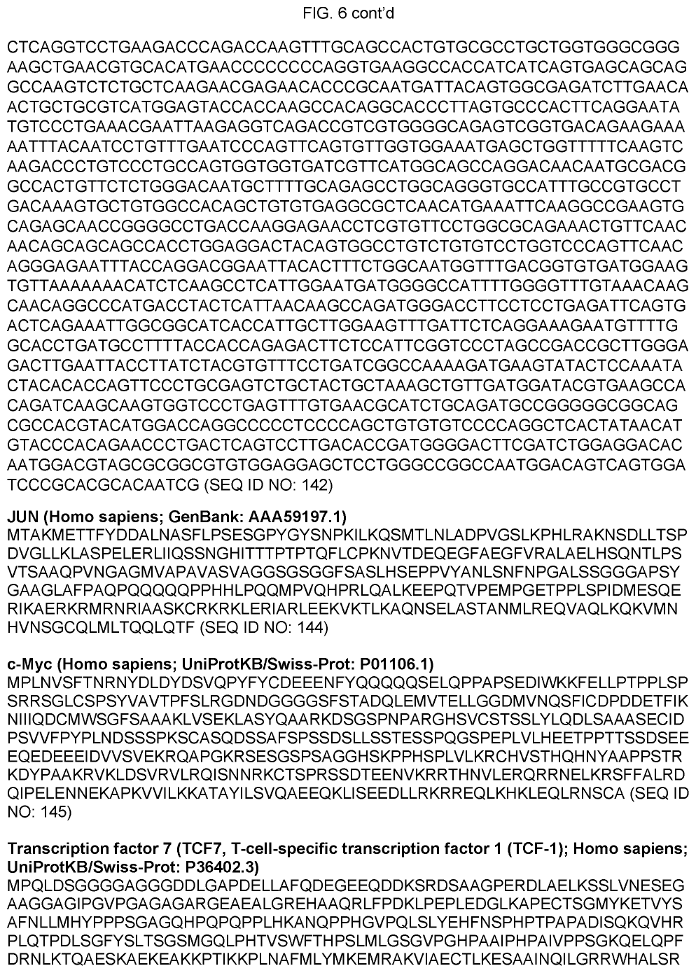

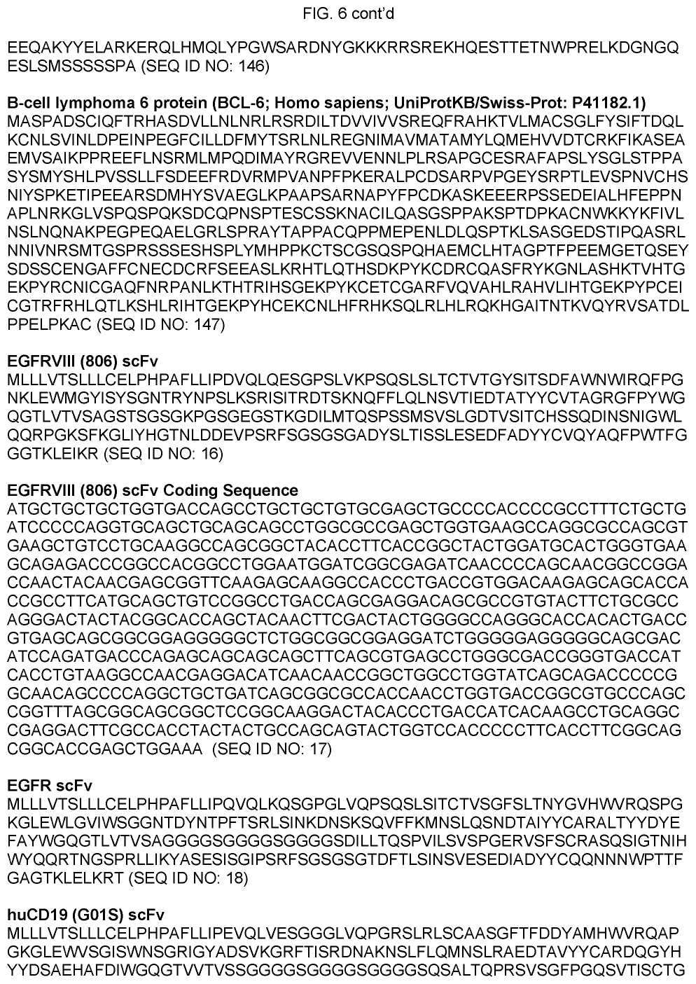

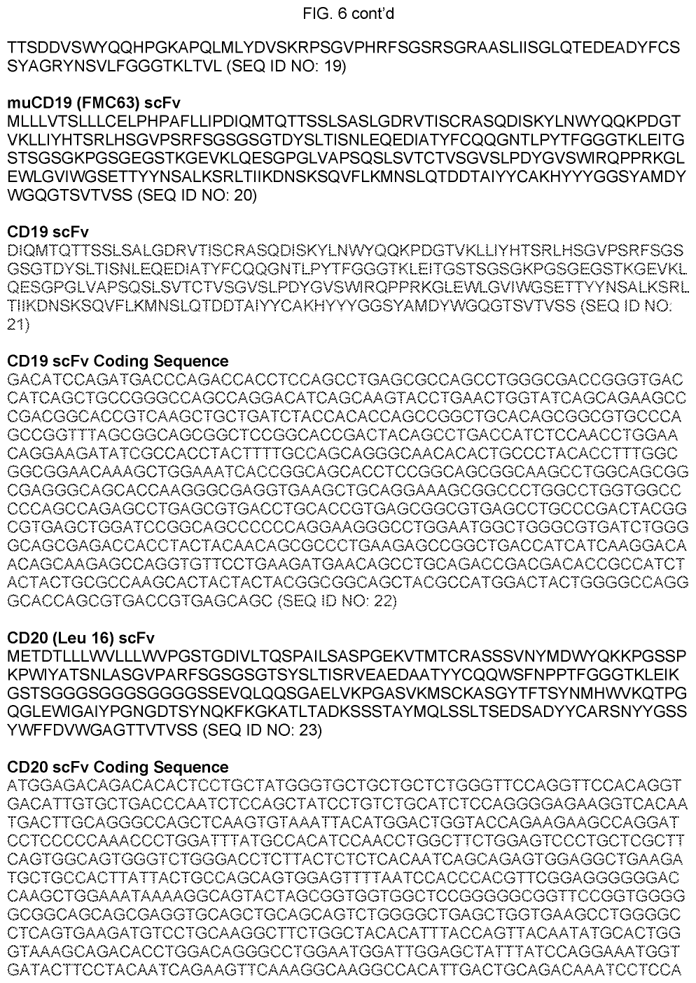

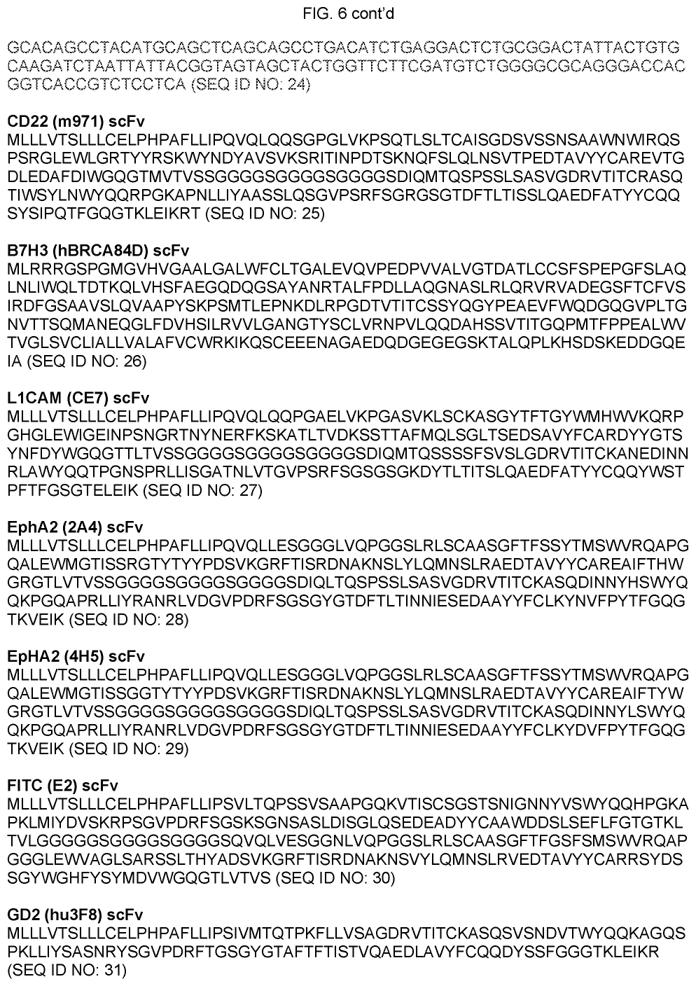

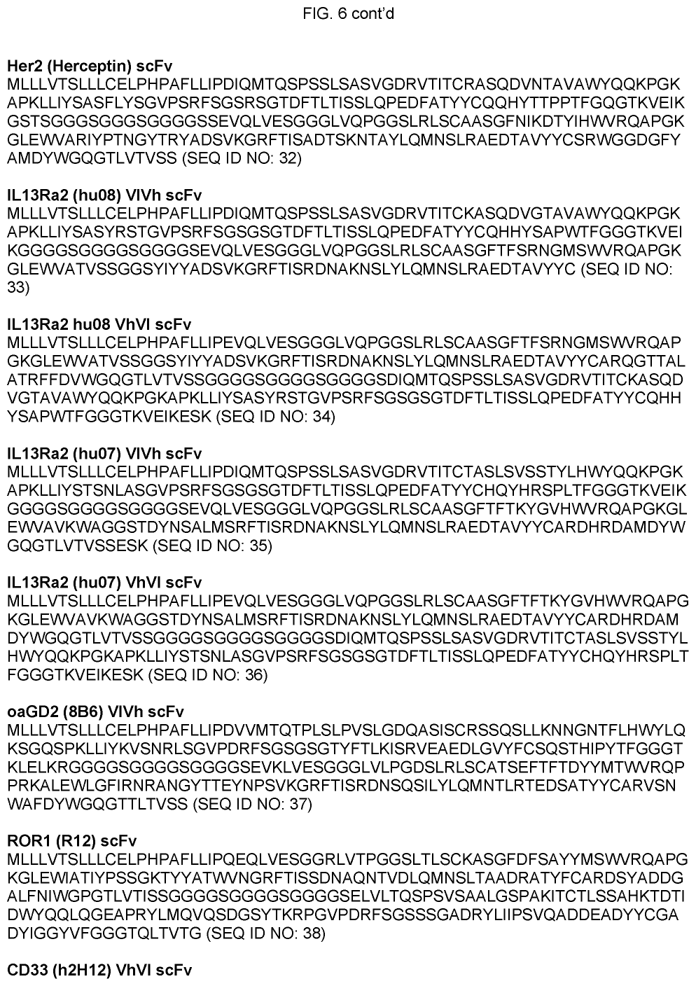

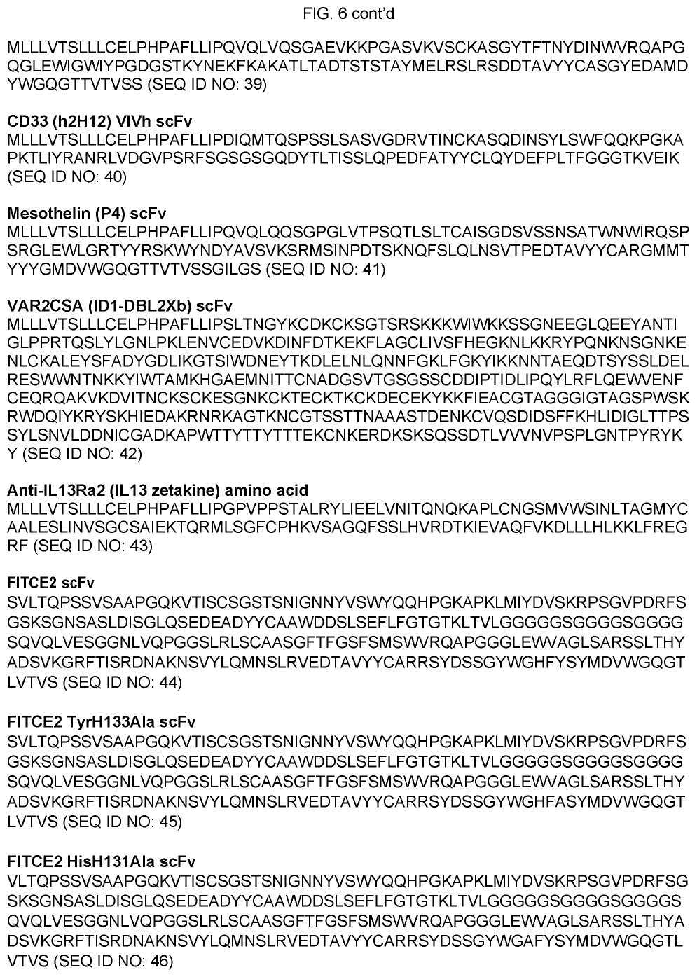

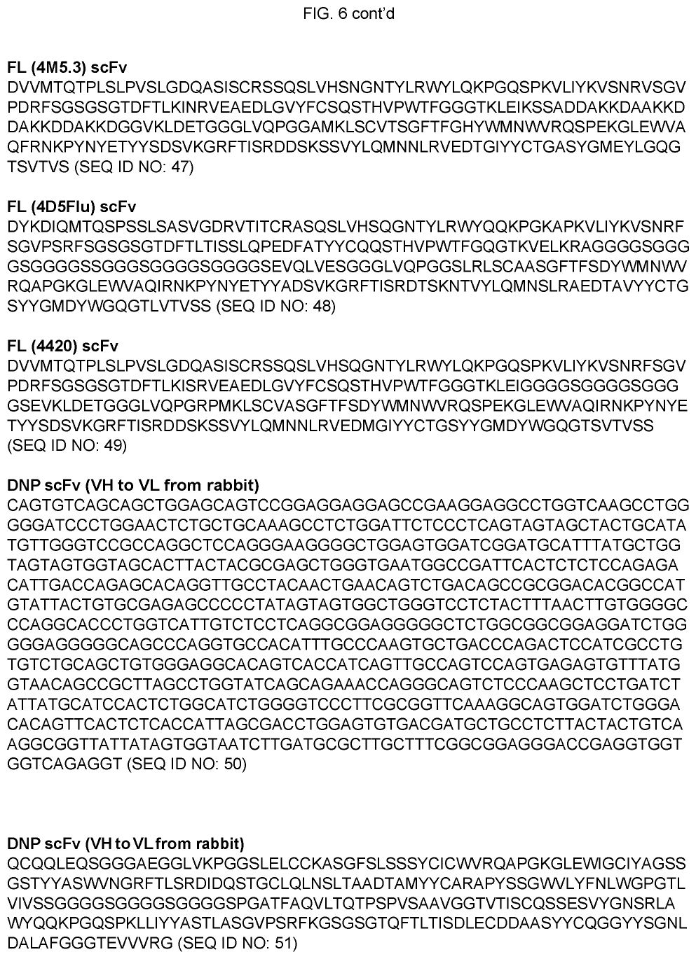

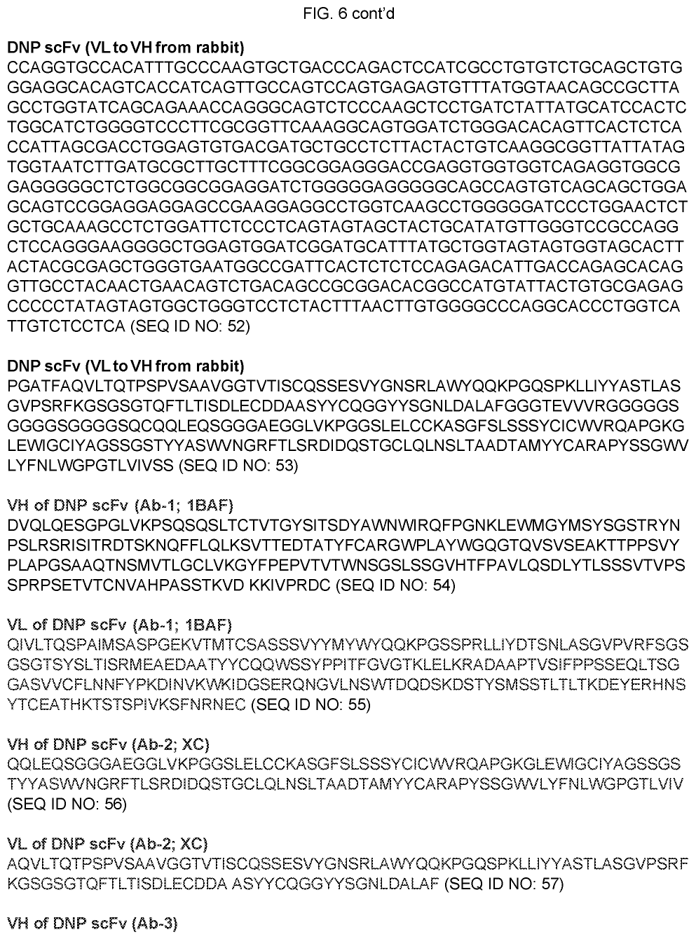

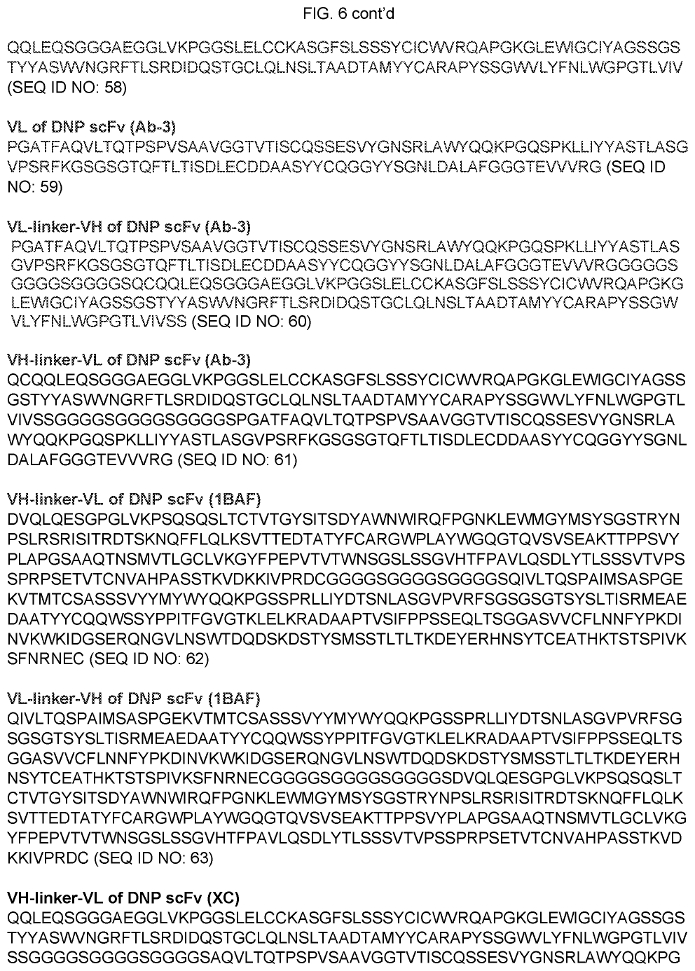

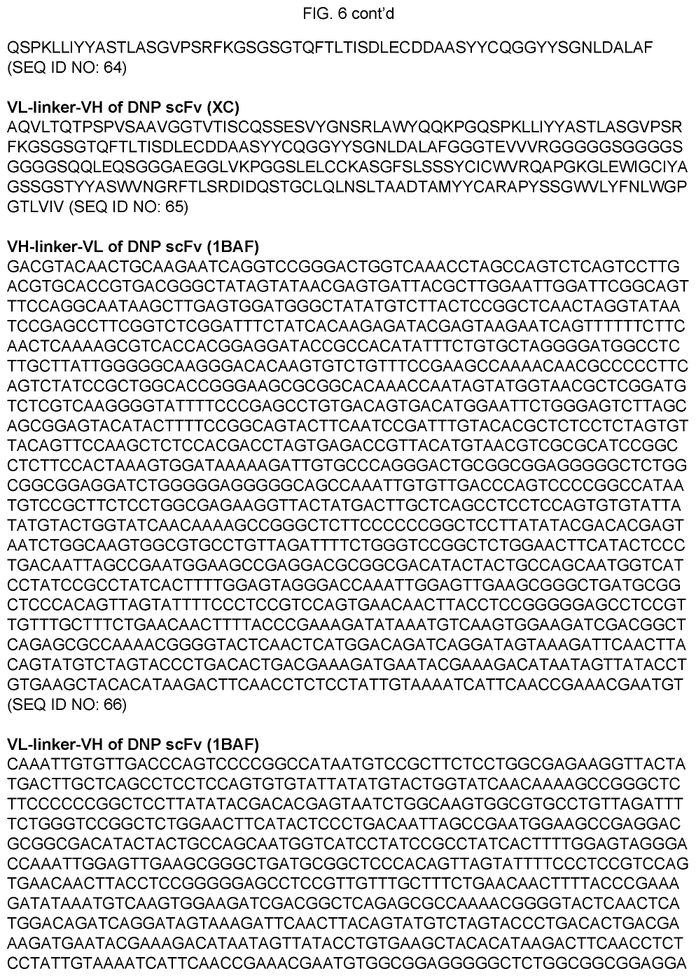

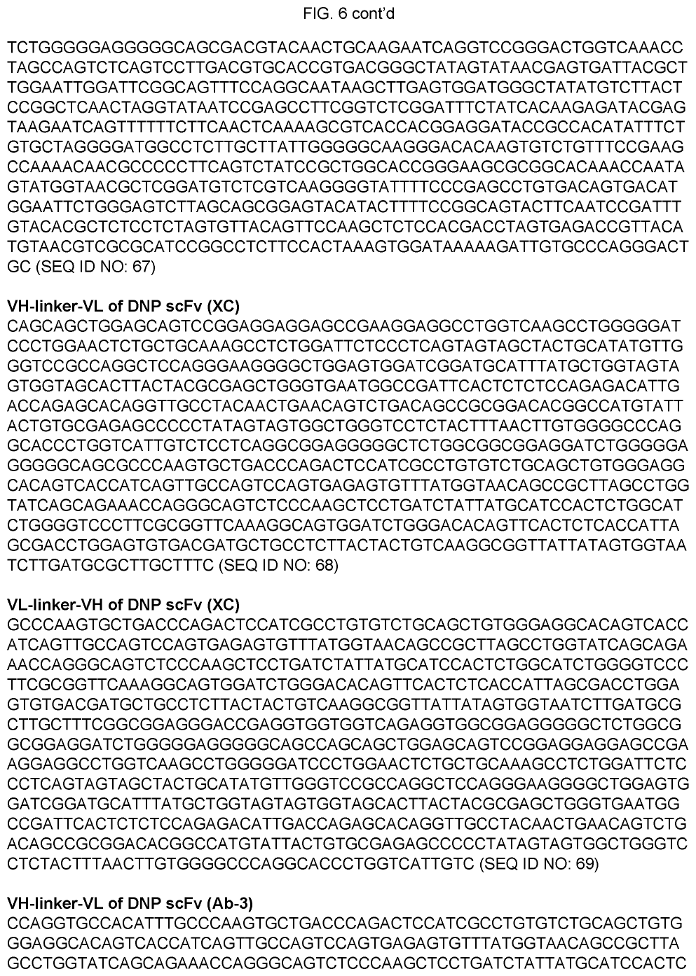

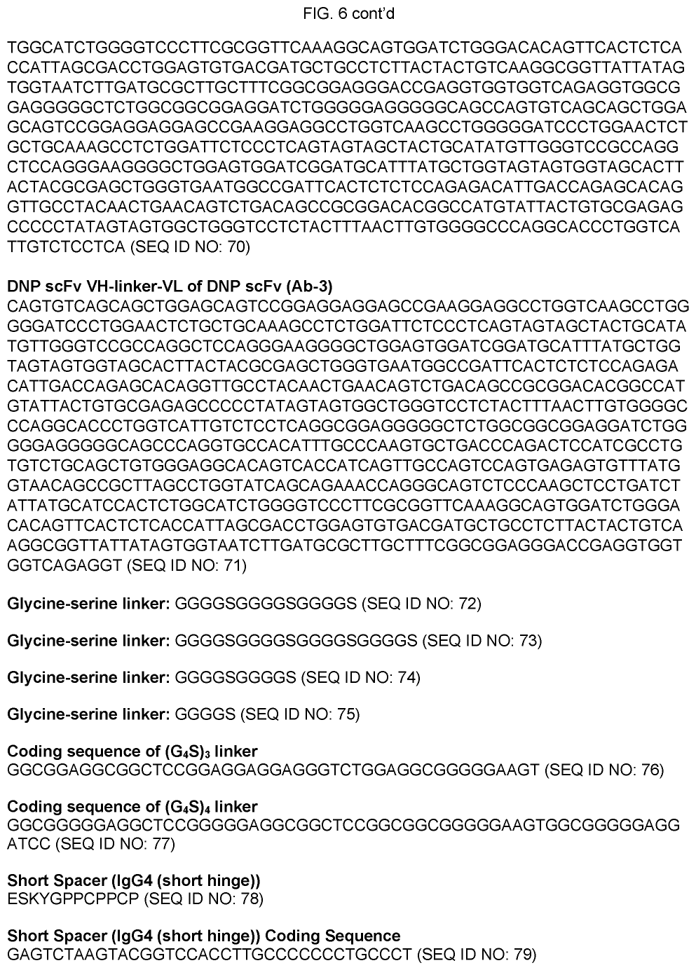

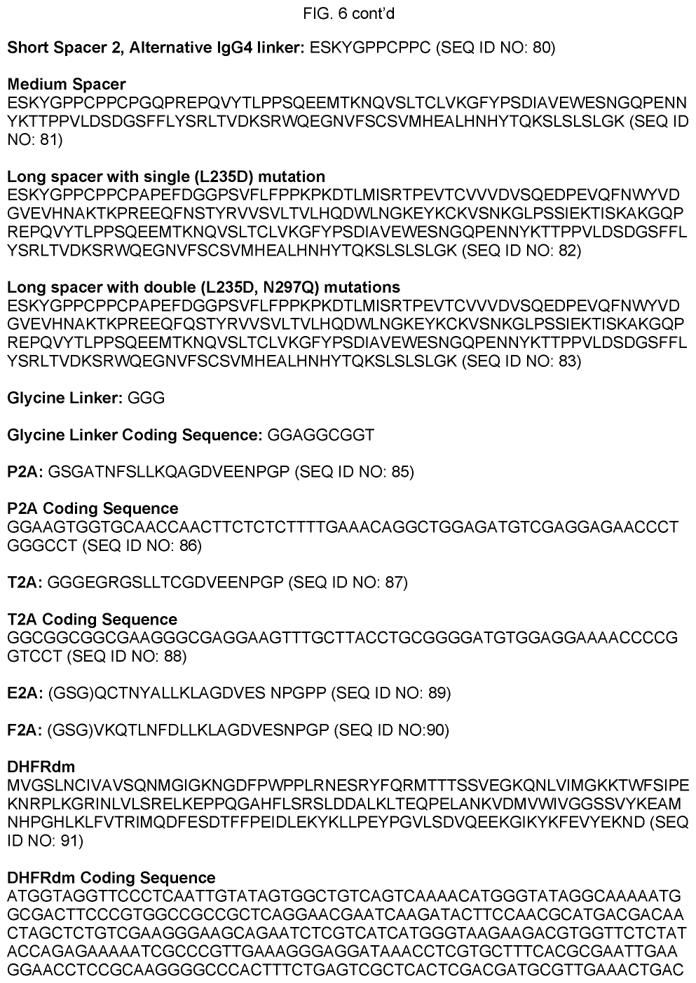

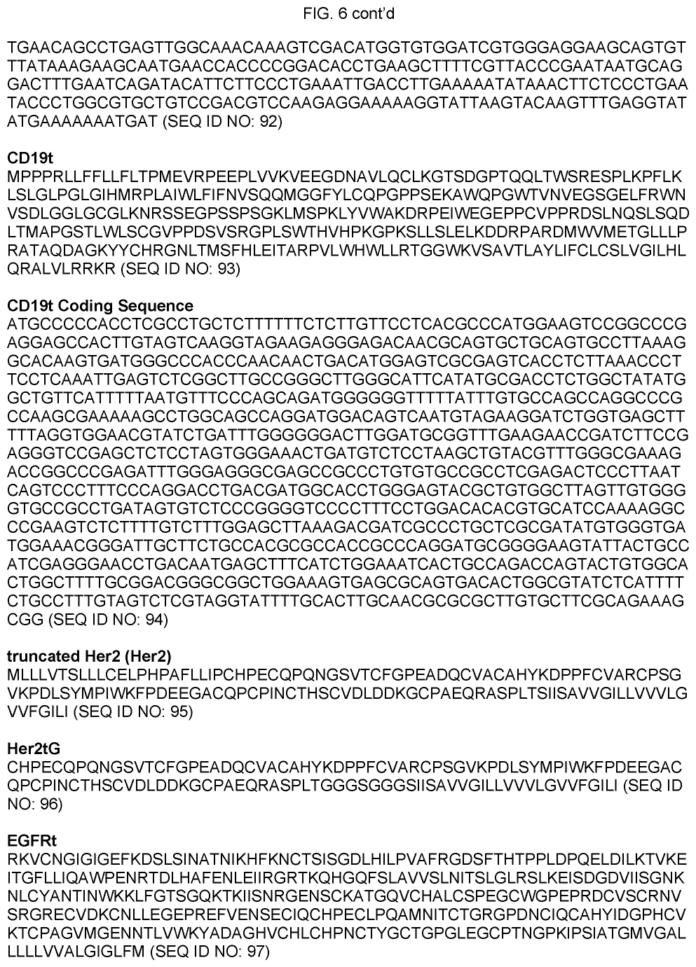

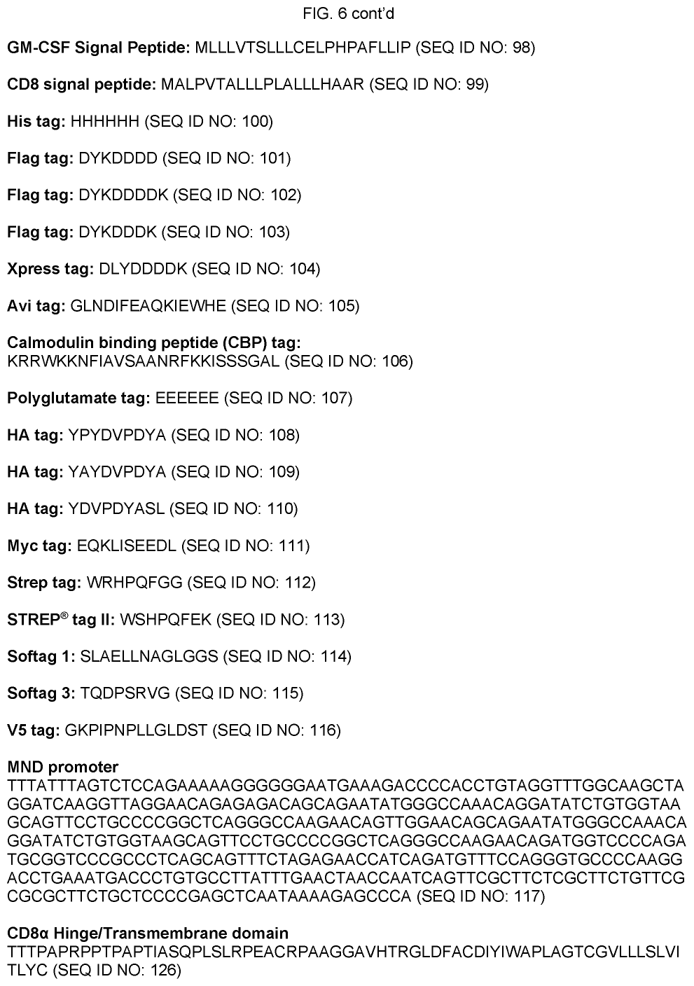

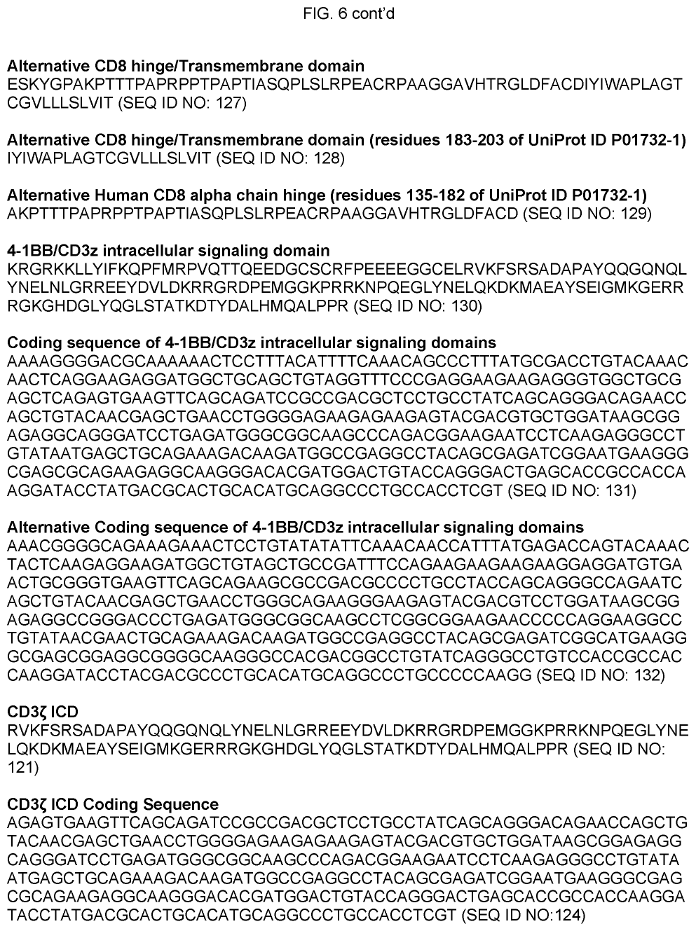

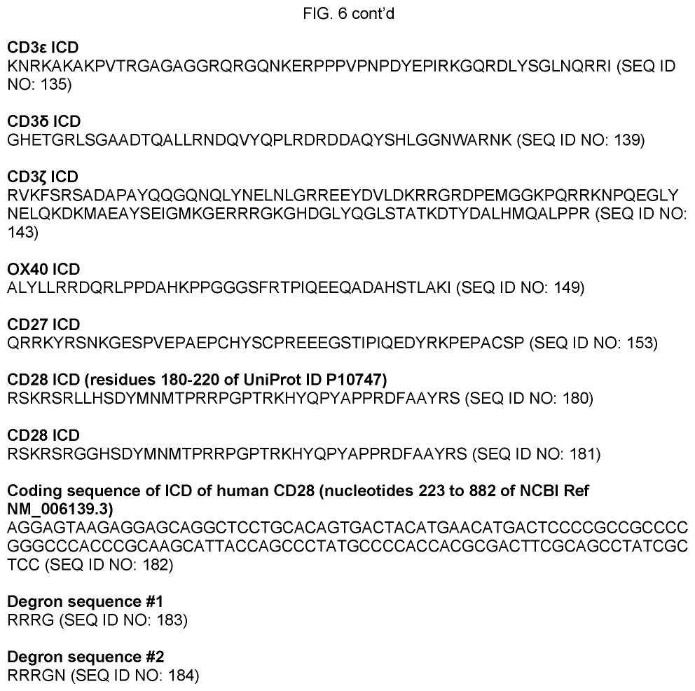

Particular embodiments utilize tamoxifen, 4-OHT, ES8, or CMP8 as the drug molecule (see, e.g., FIG. 6). Particular embodiments utilize fulvestrant or raloxifene as the drug molecule

Exemplary hormone binding domains include the estrogen receptor having at least one mutation that reduces or eliminates binding to endogenous estrogen/estradiol. The protein sequence of the estrogen receptor is provided in FIG. 6 as SEQ ID NO: 1. The ER point mutation (G521R (SEQ ID NO: 3)) ablates binding to endogenous estrogen but confers nanomolar specificity to the tamoxifen metabolite 4-OHT, fulvestrant, and other estrogen analogs. Particular embodiments herein utilize a G521R estrogen receptor binding domain (EBD) as set forth in SEQ ID NO: 4. Certain embodiments utilize a E353A mutated EBD (SEQ ID NO: 6) with the drug molecule ES8 as described in Shi & Koh, Chemistry & Biology 8 (2001) 501-510. Other embodiments can utilize EBD with 2-point mutations (L384M and M421G (SEQ ID NO: 9)) or 3-point mutations (L384M, M421G, and G521R (SEQ ID NO: 11)) as described in Gallinari et al., Chemistry & Biology, Vol. 12, 883-893 (2005) with the drug molecule CMP8. Mutations (G400V, M543A, and L544A (SEQ ID NO: 7)) also abolish estradiol binding but permit binding to tamoxifen metabolites and other estrogen analogues. Accordingly, some embodiments utilize an EBD having the sequence as set forth in SEQ ID NO: 13. For additional information regarding ER-based drug systems, see Indra et al., Nucleic Acids Research, 1999, Vol. 27, No. 22 (4324-4327) and Giacomello et al., Int. J. Dev. Biol. 45:833-838 (2001).

In some alternatives, an effective amount of the drug for allowing transcription factor activity is an amount that provides for an increase in gene expression over uninduced and/or basal activity. In other alternatives, an effective amount of the drug allows protein translation and cell activation over uninduced and/or basal activity. These amounts can be determined using known dosages and pharmacokinetic profiles of the drug. Gene expression, protein translation, and/or cell activation can be assessed utilizing measures well-known to those of ordinary skill in the art. In certain embodiments, an increase in gene expression, protein translation, and/or cell activation is an increase of at least 10%, at least 20%, at least 30%, at least 40% or at least 50%. Certain embodiments described herein can also reduce gene expression, protein translation, and/or cell activation. In these embodiments, the decrease in gene expression, protein translation, and/or cell activation is a decrease of at least 10%, at least 20%, at least 30%, at least 40% or at least 50%.

Further, in addition to the examples provided herein, other drugs can be selected based on safety record, favorable pharmacokinetic profile, tissue distribution, a low partition coefficient between the extracellular space and cytosol, and/or low toxicities.

(iii) Activity-Inducible Fusion Proteins. In particular embodiments, a fusion protein includes a transcription factor linked to an hsp90 binding domain.

In particular embodiments, the transcription factor is selected from: ca-STAT3, caSTAT5a, caSTAT5b, JUN (c-Jun), c-Myc, TCF7, BCL-6, STAT6, STAT5a, STAT5b, STAT3, c-Fos, HIF-1a, Sox2, Zeb1, Bcl-2, Mcl-1, Bcl-xL, Junb, FOXP3, Max, E2F (E2F1, EN2F2, and E2F3a), AP-1, NF-kb, FOX including FOXF2 and FoxO, Sp1, Sp2, Sp3, Sp4, Sp5, Sp6 (KLF14), Sp7, Sp8, Sp9, KLF1, KLF2, KLF3, KLF4, KLF5, KLF6, KLF7, KLF8, KLF9, KLF10, KLF11, KLF12, KLF13, KLF15, KLF16, and KLF17.

In particular embodiments, the hsp90 binding domain is a mutant estrogen receptor (ER). In particular embodiments, the mutant estrogen receptor is selected from an estrogen receptor with a G521R mutation (ERG521R), an estrogen receptor with a E353A mutation (ERE353A), an estrogen receptor with L384M and M421G mutations (ERL384M/M421G), an estrogen receptor with L384M, M421G, and G521R mutations (ERL384M/M421G/G521R), or an estrogen receptor with G400V, M543A, and L544A mutations (ERT2 or ER (T2)).

In particular embodiments, the fusion protein includes the estrogen receptor ERG521R linked to a transcription factor selected from: ca-STAT3, caSTAT5a, caSTAT5b, JUN (c-Jun), c-Myc, TCF7, BCL-6, STAT6, STAT5a, STAT5b, STAT3, c-Fos, HIF-1a, Sox2, Zeb1, Bcl-2, Mcl-1, Bcl-xL, Junb, FOXP3, Max, E2F (E2F1, EN2F2, and E2F3a), AP-1, NF-kb, FOX including FOXF2 and FoxO, Sp1, Sp2, Sp3, Sp4, Sp5, Sp6 (KLF14), Sp7, Sp8, Sp9, KLF1, KLF2, KLF3, KLF4, KLF5, KLF6, KLF7, KLF8, KLF9, KLF10, KLF11, KLF12, KLF13, KLF15, KLF16, and KLF17.

In particular embodiments, the fusion protein includes the estrogen receptor ERE353A linked to a transcription factor selected from: ca-STAT3, caSTAT5a, caSTAT5b, JUN (c-Jun), c-Myc, TCF7, BCL-6, STAT6, STAT5a, STAT5b, STAT3, c-Fos, HIF-1a, Sox2, Zeb1, Bcl-2, Mcl-1, Bcl-xL, Junb, FOXP3, Max, E2F (E2F1, EN2F2, and E2F3a), AP-1, NF-kb, FOX including FOXF2 and FoxO, Sp1, Sp2, Sp3, Sp4, Sp5, Sp6 (KLF14), Sp7, Sp8, Sp9, KLF1, KLF2, KLF3, KLF4, KLF5, KLF6, KLF7, KLF8, KLF9, KLF10, KLF11, KLF12, KLF13, KLF15, KLF16, and KLF17.

In particular embodiments, the fusion protein includes the estrogen receptor ERL384M/M421G linked to a transcription factor selected from: ca-STAT3, caSTAT5a, caSTAT5b, JUN (c-Jun), c-Myc, TCF7, BCL-6, STAT6, STAT5a, STAT5b, STAT3, c-Fos, HIF-1a, Sox2, Zeb1, Bcl-2, Mcl-1, Bcl-xL, Junb, FOXP3, Max, E2F (E2F1, EN2F2, and E2F3a), AP-1, NF-kb, FOX including FOXF2 and FoxO, Sp1, Sp2, Sp3, Sp4, Sp5, Sp6 (KLF14), Sp7, Sp8, Sp9, KLF1, KLF2, KLF3, KLF4, KLF5, KLF6, KLF7, KLF8, KLF9, KLF10, KLF11, KLF12, KLF13, KLF15, KLF16, and KLF17.

In particular embodiments, the fusion protein includes the estrogen receptor ERL384M/M421G/G521R linked to a transcription factor selected from: ca-STAT3, caSTAT5a, caSTAT5b, JUN (c-Jun), c-Myc, TCF7, BCL-6, STAT6, STAT5a, STAT5b, STAT3, c-Fos, HIF-1a, Sox2, Zeb1, Bcl-2, Mcl-1, Bcl-xL, Junb, FOXP3, Max, E2F (E2F1, EN2F2, and E2F3a), AP-1, NF-kb, FOX including FOXF2 and FoxO, Sp1, Sp2, Sp3, Sp4, Sp5, Sp6 (KLF14), Sp7, Sp8, Sp9, KLF1, KLF2, KLF3, KLF4, KLF5, KLF6, KLF7, KLF8, KLF9, KLF10, KLF11, KLF12, KLF13, KLF15, KLF16, and KLF17.

In particular embodiments, the fusion protein includes the estrogen receptor ERT2 linked to a transcription factor selected from: ca-STAT3, caSTAT5a, caSTAT5b, JUN (c-Jun), c-Myc, TCF7, BCL-6, STAT6, STAT5a, STAT5b, STAT3, c-Fos, HIF-1a, Sox2, Zeb1, Bcl-2, Mcl-1, Bcl-xL, Junb, FOXP3, Max, E2F (E2F1, EN2F2, and E2F3a), AP-1, NF-kb, FOX including FOXF2 and FoxO, Sp1, Sp2, Sp3, Sp4, Sp5, Sp6 (KLF14), Sp7, Sp8, Sp9, KLF1, KLF2, KLF3, KLF4, KLF5, KLF6, KLF7, KLF8, KLF9, KLF10, KLF11, KLF12, KLF13, KLF15, KLF16, and KLF17.

In particular embodiments, the fusion protein includes the estrogen receptor ERT2 linked to a transcription factor selected from: ca-STAT3, caSTAT5a, caSTAT5b, AP-1, c-Myc, TCF7, BCL-6, STAT6, STAT5a, STAT5b, or STAT3.

In particular embodiments, the fusion protein includes the estrogen receptor ERT2 linked to a transcription factor selected from: human ca-STAT3, human ca-STAT5a, human ca-STAT5b, or human ca-STAT6.

Linkers or spacers can be used to connect the domains of a fusion protein. In particular embodiments, linkers include linker sequence with the amino acids glycine and serine (Gly-Ser linkers). In particular embodiments, linkers include the linker sequence including (Gly₄Ser)ₙ (SEQ ID NO: 148), (Gly₃Ser)ₙ (SEQ ID NO: 150), (GGGG)ₙ (SEQ ID NO: 151), (GGG)ₙ, or (GSAGSAAGSGEF), (SEQ ID NO: 152) wherein n is an integer of 1, 2, 3, 4, 5, 6, 7, 8, 9, or 10. In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₃Ser), (Gly₄Ser), (SEQ ID NO: 154), (Gly₃Ser), (Gly₂Ser) n (SEQ ID NO: 155), or (Gly₃Ser), (Gly₄Ser)₁ (SEQ ID NO: 156). In particular embodiments, the linker is (Gly₄Ser)₄ (SEQ ID NO: 158), (Gly₄Ser)₃ (SEQ ID NO: 159), (Gly₄Ser)₂ (SEQ ID NO: 160), (Gly₄Ser)₁ (SEQ ID NO: 162), (Gly₃Ser)₂ (SEQ ID NO: 163), (Gly₃Ser)₁ (SEQ ID NO: 164), (Gly₂Ser)₂ (SEQ ID NO: 166) or (Gly₂Ser)₁, GGSGGGSGGSG (SEQ ID NO: 167), GGSGGGSGSG (SEQ ID NO: 168), or GGSGGGSG (SEQ ID NO: 170).

Junction amino acids can be present as a linker to connect the domains of fusion protein. In particular embodiments, junction amino acids are 9 amino acids or less (e.g., 1, 2, 3, 4, 5, 6, 7, 8, or 9 amino acids). In particular embodiments, a glycine-serine doublet can be used as a suitable junction amino acid linker. In particular embodiments, a single amino acid, e.g., an alanine, a glycine, can be used as a suitable junction amino acid. Gly3 as a junction amino acid sequence can also be used.

In particular embodiments, a fusion protein can include one or more tags and/or be expressed with one more selectable markers. Exemplary tags include His tag, Flag tags, Xpress tag, Avi tag, Calmodulin binding peptide (CBP) tag, Polyglutamate tag, HA tags, Myc tag, Strep tag (which refers to the original STREP® tag, STREP® tag II (IBA Institut fur Bioanalytik, Germany); see, e.g., U.S. Pat. No. 7,981,632), Softag 1, Softag 3, and V5. See FIG. 6 for exemplary sequences.

Conjugate binding molecules that specifically bind tag sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies, and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal, and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

In particular embodiments, one or more transduction markers can be co-expressed with the fusion protein, for example, using a skipping element or IRES site that allows expression of the transduction marker and other components of the fusion protein as distinct molecules. Exemplary self-cleaving polypeptides include 2A peptides from porcine teschovirus-1 (P2A), Thosea asigna virus (T2A), equine rhinitis A virus (E2A), and foot-and-mouth disease virus (F2A) (see, e.g., FIG. 6).

In particular embodiments, the transduction marker can include any cell surface displayed marker that can be detected with an antibody that binds to that marker and allows sorting of cells that have the marker. In particular embodiments, the transduction marker can include the magnetic sortable marker streptavidin binding peptide (SBP) displayed at the cell surface by a truncated Low Affinity Nerve Growth Receptor (LNGFRF) and one-step selection with streptavidin-conjugated magnetic beads (Matheson et al. (2014) PloS one 9 (10): e111437) or a truncated human epidermal growth factor receptor (EGFR) (tEGFR; see Wang et al., Blood 118:1255, 2011).

In some alternatives, the transduction marker is a truncated EGFR (EGFRt), a truncated Her2 (Her2tG), a truncated CD19 (CD19t), or the transduction marker DHFRdm.

Transduction markers can include any suitable fluorescent protein including: blue fluorescent proteins (e.g., BFP, eBFP, eBFP2); cyan fluorescent proteins (e.g., eCFP, Cerulean, CyPet); green fluorescent proteins (e.g., GFP-2, tagGFP, turboGFP, eGFP,); orange fluorescent proteins (e.g., mOrange, mKO, Kusabira-Orange); red fluorescent proteins (e.g., mKate, mPlum, DsRed monomer, mCherry, mRFP1, DsRed-Express); yellow fluorescent proteins (e.g., YFP, eYFP, Citrine, Venus); and any other suitable fluorescent proteins, including, for example, firefly luciferase.

(iv) Chimeric Antigen Receptors. Activity-inducible fusion proteins disclosed herein can optionally be co-expressed with a chimeric antigen receptor (CAR). As used herein, CAR include a synthetically designed protein including a ligand binding domain that binds to an antigen associated with a disease or disorder. The ligand binding domain is linked to one or more intracellular signaling domains of an immune cell.

(iv-a) Ligand Binding Domains. In particular embodiments, an extracellular ligand binding domain is any molecule capable of specifically binding a target antigen. Exemplary ligand binding domains include antibody binding fragments (e.g., scFv), receptors (e.g., T cell receptors), and receptor ligands (e.g., a cytokine or chemokine).

As is understood by those of ordinary skill in the art, a complete antibody includes two heavy chains and two light chains. Each heavy chain consists of a variable region and a first, second, and third constant region, while each light chain consists of a variable region and a constant region. Mammalian heavy chains are classified as α, δ, ε, γ, and μ, and mammalian light chains are classified as λ or κ. Immunoglobulins including the α, δ, ε, γ, and μ heavy chains are classified as immunoglobulin (lg) A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains μ and ε have a constant region composed of four immunoglobulin domains. The second and third constant regions are referred to as “CH2 domain” and “CH3 domain”, respectively. Each arm of the γ includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity determining regions” or “CDRs”.

CDR sets can be based on, for example, Kabat numbering (Kabat et al. (1991) “Sequences of Proteins of Immunological Interest,” 5th Ed. Public Health Service, National Institutes of Health, Bethesda, Md. (“Kabat” numbering scheme)); Chothia (Al-Lazikani et al. (1997) JMB 273:927-948 (“Chothia” numbering scheme)); Martin (Abinandan et al. (2008) Mol Immunol. 45:3832-3839 (“Martin” numbering scheme)); Gelfand (Gelfand and Kister (1995) Proc Natl Acad Sci USA. 92:10884-10888; Gelfand et al. (1998) Protein Eng. 11:1015-1025; Gelfand et al. (1996) Proc Natl Acad Sci USA. 93:3675-3678; Gelfand et al. (1998) J Comput Biol. 5:467-477 (“Gelfand” numbering scheme)); Contact (MacCallum et al. (1996) J. Mol. Biol. 262:732-745 (Contact numbering scheme)); IMGT (Lefranc et al. (2003) Dev Comp Immunol 27 (1): 55-77 (“IMGT” numbering scheme)); AHo (Honegger and Plückthun (2001) J Mol Biol 309 (3): 657-670 (“AHo” numbering scheme)); North (North et al. (2011) J Mol Biol. 406 (2): 228-256 (“North” numbering scheme)); or other numbering schemes.

Software programs and bioinformatical tools, such as ABodyBuilder (Leem et al. (2016) MAbs 8 (7): 1259-1268), PIGSPro (Lepore et al. (2017) Nucleic Acids Res 45 (W1): W17-W23), Kotai Antibody Builder (Yamashita et al. (2014) Bioinformatics 30 (22): 3279-3280), Rosetta Antibody (Weitzner et al. (2017) Nature Protocols 12:401-416), Paratome (Kunik et al. (2012) Nucleic Acids Res 40: W521-W524), Antibody i-Patch (Krawczyk et al. (2013) Protein Eng Des Sel 26 (10): 621-629), and proABC-2 (Ambrosetti et al. (2020) Bioinformatics 36 (20): 5107-5108 can also be used to determine CDR sequences.

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain. References to “V_{L}” or “VL” refer to the variable region of an immunoglobulin light chain.

Antibodies that specifically bind a cell surface molecule can be prepared using methods of obtaining monoclonal antibodies, methods of phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined. Many relevant antibodies are also publicly known and commercially available.

In some alternatives, antibodies specifically bind to a cancer cell or virally-infected cell surface molecule and do not cross react with nonspecific components such as bovine serum albumin or other unrelated antigens.

The term “antibody fragment” refers to at least one portion of an antibody, that retains the ability to specifically binding an antigen. Examples of antibody fragments include Fab, Fab′, F(ab′)₂, Fv fragments, single chain variable (scFv) antibody fragments, disulfide-linked Fvs (sdFv), a Fd fragment including VH and constant CH1 domains, linear antibodies, single domain antibodies such as sdAb (either VL or VH), camelid variable heavy only (VHH) domains, multi-specific antibodies formed from antibody fragments such as a bivalent fragment including two Fab fragments linked by a disulfide bridge at the hinge region, and an isolated CDR or other epitope binding fragments of an antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). An antigen binding fragment can also be incorporated into single domain antibodies, maxibodies, minibodies, intrabodies, diabodies, triabodies, tetrabodies, v-NAR and bis-scFv (see, e.g., Hollinger and Hudson (2005) Nature Biotechnology 23:1126-1136).

In particular embodiments, a binding domain can include humanized forms of non-human (e.g., murine) antibodies or antigen binding fragments thereof. A humanized antibody includes an antibody in which the constant and variable framework region of one or more human immunoglobulins is fused with the binding region, e.g., the CDR, of an animal (non-human) immunoglobulin. Such humanized antibodies are designed to maintain the binding specificity of the non-human antibody from which the binding regions are derived but avoid an immune reaction against the non-human antibody. In particular embodiments, a binding domain can include a fully human antibody or antibody fragment thereof, where the whole molecule is of human origin or includes an amino acid sequence identical to a human form of the antibody or immunoglobulin.

The term “scFv” refers to an engineered fusion protein including the VH and VL of an antibody linked via a linker and capable of being expressed as a single chain polypeptide. The scFv retains the specificity of the intact antibody from which it is derived. In particular embodiments, a linker connecting the variable regions can include glycine-serine linkers, including, for example, those shown as SEQ ID NOs: 72-75 or described elsewhere herein. In particular embodiments, an scFv may have the VL and VH variable regions in either order, e.g., with respect to the N-terminal and C-terminal ends of the polypeptide, the scFv may include VL-linker-VH or may include VH-linker-VL.

There are also numerous ways to identify and select particular TCR for use within CAR. For example, the sequences of numerous TCR that bind particular antigen fragments are known and publicly available.

TCR can also be identified for use with a particular antigen by, for example, isolating T cells that bind a particular antigen/MHC complex and sequencing the TCR chains binding the complex. TCR genes encoding TCR can be readily cloned by, for example, the 5′ RACE procedure using primers corresponding to the sequences specific to the TCR α-chain gene and the TCR β-chain gene.

In particular embodiments, it may be necessary to pair TCR chains following sequencing (i.e., to perform paired chain analysis). Various methods can be utilized to pair chains, when necessary. For example, chain pairing may be assisted in silico by computer methods, such as immunology gene alignment software available from IMGT, JOINSOLVER, VDJSolver, SoDA, iHMMune-align, or other similar tools for annotating VDJ gene segments. Assays such as PairSEQ® (Adaptive Biotechnologies Corp., Seattle, WA) have also been developed.

In particular embodiments, an engineered TCR includes a single chain T cell receptor (scTCR) including Vα/β and Cα/β chains (e.g., Vα-Cα, Vβ-Cβ, Vα-Vβ) or including Vα-Cα, Vβ-Cβ, Vα-Vβ pair specific for a target of interest (e.g., peptide-MHC complex).

Cancer antigens are proteins that are produced by cancer cells and viral antigens are proteins produced by virally-infected cells. Ligand binding domains of CAR disclosed herein can be selected to bind cancer antigens or viral antigens. In some alternatives, cancer or viral antigens are selectively expressed or overexpressed on the cancerous or infected cells as compared to other cells of the same tissue type. In some alternatives, a cancer or viral antigen is a cell surface molecule that is found on cancer cells or virally-infected cells and is not substantially found on normal tissues, or restricted in its expression to non-vital normal tissues.

Exemplary cancer antigens include carcinoembryonic antigen (CEA), prostate specific antigen, Prostate Stem Cell antigen (PSCA), PSMA, Her2/neu, estrogen receptor, progesterone receptor, ephrinB2, CD19, CD20, CD22, CD23, CD123, CS-1, CE7, hB7H3, ROR1, mesothelin, c-Met, GD-2, MAGE A3 TCR, EGFR, EGFRvlll, EphA2, IL13Ra2, L1CAM, oaGD2, GD2, B7H3, CD33, FITC, VAR2CSA, MUC16, PD-L1, ERBB2, folate receptor (FOLR), CD56; glypican-2, disialoganglioside, EpCam, L1-CAM, Lewis Y, WT-1, Tyrosinase related protein 1 (TYRP1/gp75); GD2, B-cell maturation antigen (BCMA), CD24, SV40 T, carboxy-anhydrase-IX (CAIX); and CD133 Other examples are known to those of ordinary skill in the art.

Particular embodiments utilize ligand binding domains that specifically bind HER2, CE7, hB7H3, EGFR, EGFRvIII, CD19, CD20, CD22, EphA2, IL13Ra2, L1CAM, oaGD2, B7H3, CD33, Mesothelin, ROR1, FITC or VAR2CSA.

In particular embodiments, an scFv utilized with the teaching of this disclosure includes an huCD19 (G01S) scFv, a muCD19 (FMC63) scFv, a CD20 (Leu 16) scFv, a CD22 (m971) scFv, a B7H3 (hBRCA84D) scFv, an L1CAM (CE7) scFv, an EGFR scFv, an EGFRVIII (806) scFv, an EphA2 (2A4) scFv, an EpHA2 (4H5) scFv, an FITC (E2) scFv, a GD2 (hu3F8) scFv, a Her2 (Herceptin) scFv, an IL13Ra2 (hu08) VIVh scFv, an IL 13Ra2 hu08 VhV1 scFv, an IL13Ra2 (hu07) VhV1 scFv, an IL13Ra2 (hu07) VhVI scFv, an oaGD2 (8B6) VIVh, a ROR1 (R12) scFv, a CD33 (h2H12) VhVI scFv, a CD33 (h2H12) VIVh scFv, a mesothelin (P4) scFv, a VAR2CSA (ID1-DBL2Xb) scFv, or an IL13Ra2 (IL13 zetakine) amino acid sequence. See FIG. 10 for sequences of these exemplary scFv.

Binding domains that bind the following exemplary viral antigens can also be used: coronaviral antigens: the spike(S) protein; cytomegaloviral antigens: envelope glycoprotein B and CMV pp65; Epstein-Barr antigens: EBV EBNAI, EBV P18, and EBV P23; hepatitis antigens: the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens: immediate early proteins and glycoprotein D; HIV antigens: gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens: hemagglutinin and neuraminidase; Japanese encephalitis viral antigens: proteins E, M-E, M-E-NS1, NS1, NS1-NS2A and 80% E; measles antigens: the measles virus fusion protein; rabies antigens: rabies glycoprotein and rabies nucleoprotein; respiratory syncytial viral antigens: the RSV fusion protein and the M2 protein; rotaviral antigens: VP7sc; rubella antigens: proteins E1 and E2; and varicella zoster viral antigens: gpl and gpll. See Fundamental Virology, Second Edition, eds. Fields, B. N. and Knipe, D. M. (Raven Press, New York, 1991) for additional examples of viral antigens.

In particular embodiments, the binding domain is specific for a B-cell ligand, wherein the binding domain is specific for CDId, CD5, CD19, CD20, CD21, CD22, CD23/Fc epsilon RII, CD24, CD25/IL-2 R alphaCD27/TNFRSF7, CD32, CD34, CD35, CD38, CD40 (TNFRSF5), CD44, CD45, CD45.1, CD45.2, CD54 (ICAM-1), CD69, CD72, CD79, CD80, CD84/SLAMF5, LFA-1, CALLA, BCMA, B-cell receptor (BCR), IgMs, IgD, B220/CD45R, Clq R1/CD93, CD84/SLAMF5, BAFF R/TNFRSF13C, B220/CD45R, B7-1/CD80, B7-2/CD86, TNFSF7, TNFRSF5, ENPP-1, HVEM/TNFRSF14, BLIMP 1/PRDMI, CXCR4, DEP-1/CD148 or EMMPRIN/CD147.

CAR binding domains can also bind other immune cell antigens found on, e.g., natural killer T (NKT) cells, natural killer cells (also known as K cells and killer cells), tumor-infiltrating lymphocytes (TILs), marrow-infiltrating lymphocytes (MILs), MAIT cells, macrophages, monocytes, and/or dendritic cells. These cells and exemplary cell surface antigens are described elsewhere herein.

Binding domains described herein can also bind haptens. Haptens include any small molecule which, when combined with a larger carrier such as a protein, elicits the production of antibodies which bind specifically to it (in the free or combined state). Haptens can include peptides, other larger chemicals, and aptamers. In some embodiments, a hapten can be any hapten provided in the hapten database accessible on the World Wide Web under the URL crdd.osdd.net/raghava/haptendb/.

In some embodiments, a hapten is fluorescein, urushiol, quinone, biotin, or dinitrophenol, and/or derivatives thereof. Exemplary scFv that bind hapten are provided in FIG. 6 and include forms of FITCE2 scFv, FITCE2 TyrH133Ala scFv, FITCE2 HisH131Ala scFv, FL (4M5.3) scFv, FL (4D5Flu) scFv, FL (4420) scFv, and DNP scFv.

In particular embodiments, the binding domain can target a small molecule ligand linked to a targeting moiety. In particular embodiments, a small molecule ligand includes a folate, DUPA, an NK-1R ligand, a CAIX ligand, a ligand of gamma glutamyl transpeptidase, an NKG2D ligand, or a CCK2R ligand, each of which is a small molecule ligand that binds specifically to cancer cells (i.e., the receptor for these ligands is overexpressed on cancers compared to normal tissues). In particular embodiments, the targeting moiety includes fluorescein, fluorescein isothiocyanate (FITC), NHS and/or fluorescein. In particular embodiments, the binding domain is specific for the targeting moiety. In particular embodiments, the binding domain includes an E2 anti-fluorescein antibody or antibody fragment.

(iv-b) Intracellular Signaling Domains. An intracellular component of a protein includes one or more intracellular signaling domains. In particular embodiments, the intracellular signaling domain generates a signal that promotes an immune effector function of a CAR modified cell. In particular embodiments, the intracellular signaling domain generates a stimulatory and/or co-stimulatory signal based on ligand binding. Examples of immune effector function include cytolytic activity and helper activity, including the secretion of cytokines. Intracellular signaling domain signals can also lead to immune cell proliferation, activation, differentiation, and the like.

A signaling domain refers to the functional portion of a protein which acts by transmitting information within the cell to regulate cellular activity via defined signaling pathways by generating second messengers or functioning as effectors by responding to such messengers. Stimulation refers to a primary response induced by binding of a stimulatory molecule (e.g., a CAR) or co-stimulatory molecule with its cognate ligand, thereby mediating a signal transduction event, such as signal transduction via appropriate signaling domains of the CAR. Stimulation can mediate altered expression of certain molecules.

An intracellular signaling domain can include the entire intracellular portion of the signaling domain or a functional fragment thereof. In particular embodiments, an intracellular signaling domain can include a primary intracellular signaling domain. In particular embodiments, primary intracellular signaling domains include those derived from the molecules responsible for primary stimulation, or antigen dependent stimulation. In particular embodiments, the intracellular signaling domain can include a costimulatory intracellular domain.

A primary intracellular signaling domain can include a signaling motif which is known as an immunoreceptor tyrosine-based activation motif or ITAM. Examples of ITAM containing primary cytoplasmic signaling sequences include those derived from CD3ζ, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

In particular embodiments, a CD3ζ (CD247) stimulatory domain can include amino acid residues from the cytoplasmic domain of the T cell receptor zeta chain, or functional fragments thereof, that are sufficient to functionally transmit an initial signal necessary for cell activation. In particular embodiments, a CD3Z stimulatory domain can include a human CD3Z stimulatory domain or functional fragments thereof. In particular embodiments, a CD34 stimulatory domain includes SEQ ID NO: 121. In particular embodiments, a CD32 stimulatory domain is encoded by SEQ ID NO: 124. In particular embodiments, in the case of an intracellular signaling domain that is derived from a CD37 molecule, the intracellular signaling domain retains sufficient CD3Z structure such that it can generate a signal under appropriate conditions.

In particular embodiments, the intracellular signaling domain can include a costimulatory intracellular domain. In particular embodiments, costimulatory intracellular signaling domains include those derived from molecules responsible for costimulatory signals, or antigen independent stimulation. In particular embodiments, a costimulatory intracellular signaling domain can be the intracellular portion of a costimulatory molecule. A costimulatory molecule refers to a cognate binding partner on an immune cell that specifically binds with a costimulatory ligand, thereby mediating a costimulatory response by the immune cell, such as proliferation. Costimulatory molecules include cell surface molecules other than antigen receptors or their ligands that contribute to an efficient immune response. A costimulatory molecule can be represented in the following protein families: TNF receptor proteins, immunoglobulin-like proteins, cytokine receptors, integrins, signaling lymphocytic activation molecules (SLAM proteins), and activating NK cell receptors. Examples of such molecules include: an MHC class I molecule, B and T cell lymphocyte attenuator (BTLA, CD272), a Toll ligand receptor, CD27, CD28, 4-1BB (CD137), OX40, GITR, CD30, CD40, ICOS (CD278), BAFFR, HVEM (LIGHTR), ICAM-1, lymphocyte function-associated antigen-1 (LFA-1; CD11a/CD18), CD2, CDS, CD7, CD287, LIGHT, NKG2C, NKG2D, SLAMF7, NKp80 (KLRF1), NKp30, NKp44, NKp46, CD160 (BY55), B7-H3 (CD276), CD19, CD4, CD8a, CD8B, IL2RB, IL2Ry, IL7Ra, ITGA4, VLA1, CD49a, IA4, CD49d, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, and the like.

In particular embodiments, a costimulatory intracellular signaling domain includes 4-1BB (CD137, TNFRSF9). 4-1BB refers to a member of the tumor necrosis factor receptor (TNFR) superfamily. In particular embodiments, a 4-1BB costimulatory domain includes a human 4-1BB costimulatory domain or a functional fragment thereof. In particular embodiments, a 4-1BB costimulatory domain includes SEQ ID NO: 120.

In particular embodiments, a costimulatory intracellular signaling domain includes CD28. CD28 is a T cell-specific glycoprotein involved in T cell activation, the induction of cell proliferation and cytokine production, and promotion of T cell survival. In particular embodiments, a CD28 costimulatory domain includes a human CD28 costimulatory domain or a functional fragment thereof. In particular embodiments, a human CD28 costimulatory domain includes SEQ ID NO: 180. In particular embodiments, a human CD28 costimulatory domain is encoded by SEQ ID NO: 182.

In particular embodiments, an intracellular signaling domain includes a combination of one or more stimulatory domains and one or more costimulatory domains described herein. In particular embodiments, an intracellular signaling domain includes a 4-1BB costimulatory domain and a CD34 stimulatory domain. In particular embodiments, an intracellular signaling domain including a 4-1BB costimulatory domain and a CD32 stimulatory domain is set forth in SEQ ID NO: 130. In particular embodiments, an intracellular signaling domain including a 4-1BB costimulatory domain and a CD32 stimulatory domain is encoded by a sequence set forth in SEQ ID NO: 132 or SEQ ID NO: 131.

(iv-c) Transmembrane Domains. CAR can be designed to include a transmembrane domain that links an extracellular component of the CAR to an intracellular component of the CAR when expressed. A transmembrane domain can anchor a CAR to a cell membrane. A transmembrane domain can include one or more additional amino acids adjacent to the transmembrane region, e.g., one or more amino acids associated with the extracellular region of the protein from which the transmembrane was derived (e.g., 1, 2, 3, 4, 5, 6, 7, 8, 9, 10, 11, 12, 13, 14, 15 amino acids, or more of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15 amino acids, or more of the intracellular region). In particular embodiments, the transmembrane domain may be from the same protein that the signaling domain, costimulatory domain, or hinge domain is derived from. In particular embodiments, the transmembrane domain is not derived from the same protein that any other domain of a fusion protein is derived from. In particular embodiments, the transmembrane domain can be selected or modified by amino acid substitution to avoid binding of or to minimize interactions with other domains in the fusion protein.

In particular embodiments, a transmembrane domain has a three-dimensional structure that is thermodynamically stable in a cell membrane, and generally ranges in length from 15 to 30 amino acids. The structure of a transmembrane domain can include an alpha helix, a beta barrel, a beta sheet, a beta helix, or any combination thereof.

The transmembrane domain may be derived either from a natural or from a recombinant source. Where the source is natural, the domain may be derived from any membrane-bound or transmembrane protein. In particular embodiments, the transmembrane domain is capable of signaling to the intracellular domain(s) whenever a fusion protein having an extracellular ligand binding domain has bound to a target. In particular embodiments, a transmembrane domain may include at least the transmembrane region(s) of: the α, β, or ζ chain of the T-cell receptor; CD28; CD27; CD38; CD45; CD4; CD5; CD8; CD9; CD16; CD22; CD33; CD37; CD64; CD80; CD86; CD134; CD137; and/or CD154. In particular embodiments, a transmembrane domain may include at least the transmembrane region(s) of: KIRDS2; OX40; CD2; LFA-1; ICOS; 4-1BB; GITR; CD40; BAFFR; HVEM; SLAMF7; NKp80; NKp44; NKp30; NKp46; CD160; CD19; IL2Rß; IL2Ry; IL7Ra; ITGA1; VLA1; CD49a; ITGA4; IA4; CD49D; ITGA6; VLA-6; CD49f; ITGAD; CDI Id; ITGAE; CD103; ITGAL; CDI Ia; ITGAM; CDI Ib; ITGAX; CDI Ic; ITGB1; CD29; ITGB2; CD18; ITGB7; TNFR2; DNAM1; SLAMF4; CD84; CD96; CEACAM1; CRT AM; Ly9; CD160; PSGL1; CD100; SLAMF6 (NTB-A, Lyl08); SLAM; BLAME; SELPLG; LTBR; PAG/Cbp; NKG2D; and/or NKG2C. In particular embodiments, a transmembrane domain may include a transmembrane domain from CD28 or the CD8a chain. In particular embodiments, a CD8 transmembrane domain includes SEQ ID NO: 126, 127, or 128.

In particular embodiments, the transmembrane domain can include predominantly hydrophobic residues such as leucine and valine. In particular embodiments, the transmembrane domain can include a triplet of phenylalanine, tryptophan and valine found at each end of the transmembrane domain. In particular embodiments, a CD28 or CD8 hinge is juxtaposed on the extracellular side of the transmembrane domain.

(iv-d) Linkers. As used herein, a linker within a CAR can be any portion of a CAR that serves to connect two subcomponents or domains of the CAR. In particular embodiments, linkers can provide flexibility for different components of the CAR. Linkers in the context of linking the domains of a fusion protein are described above. Linkers can also include spacer regions and junction amino acids. In certain examples, when a more rigid linker is required, proline-rich linkers can be used.

Spacer regions are a type of linker region that are used to create appropriate distances and/or flexibility from other linked components.

In particular embodiments, the length of a spacer region can be customized for individual purposes. For example, a spacer region can be customized for individual cellular markers on targeted cells to optimize cell recognition and destruction following fusion protein binding. In certain examples, the spacer can be of a length that provides for increased responsiveness of a CAR expressing cell following antigen binding, as compared to in the absence of the spacer. In particular embodiments, a spacer region length can be selected based upon the location of a cellular marker epitope, affinity of a binding domain for the epitope, and/or the ability of the CAR modified cells to destroy target cells ex vivo and/or in vivo in response to cellular marker recognition. Spacer regions can also allow for high expression levels in CAR modified cells. In particular embodiments, an extracellular spacer region of a CAR is located between a transmembrane domain and the extracellular binding domain.

Exemplary spacers include those having 10 to 250 amino acids, 10 to 200 amino acids, 10 to 150 amino acids, 10 to 100 amino acids, 10 to 50 amino acids, or 10 to 25 amino acids. In particular embodiments, a spacer region is 12 amino acids, 20 amino acids, 21 amino acids, 26 amino acids, 27 amino acids, 45 amino acids, or 50 amino acids. In particular embodiments, a long spacer is greater than 119 amino acids, an intermediate spacer is 13-119 amino acids, and a short spacer is 10-12 amino acids.

In particular embodiments, a spacer region includes an immunoglobulin hinge region. An immunoglobulin hinge region may be a wild-type immunoglobulin hinge region or an altered wild-type immunoglobulin hinge region. In particular embodiments, an immunoglobulin hinge region is a human immunoglobulin hinge region. An immunoglobulin hinge region may be an IgG, IgA, IgD, IgE, or IgM hinge region. An IgG hinge region may be an IgG1, IgG2, IgG3, or IgG4 hinge region. In particular embodiments, the spacer region can include all or a portion of a hinge region sequence from IgG1, IgG2, IgG3, IgG4 or IgD alone or in combination with all or a portion of a CH2 region; all or a portion of a CH3 region; or all or a portion of a CH2 region and all or a portion of a CH3 region. As used herein, a “wild type immunoglobulin hinge region” refers to a naturally occurring upper and middle hinge amino acid sequences interposed between and connecting the CH1 and CH2 domains (for lgG, IgA, and IgD) or interposed between and connecting the CH1 and CH3 domains (for IgE and IgM) found in the heavy chain of an antibody.

Exemplary spacers include IgG4 hinge alone, IgG4 hinge linked to CH2 and CH3 domains, or IgG4 hinge linked to the CH3 domain. In particular embodiments, the spacer includes an IgG4 linker as set forth in SEQ ID NOs: 78 or 80. Hinge regions can be modified to avoid undesirable structural interactions such as dimerization with unintended partners. Other examples of hinge regions that can be used in fusion proteins described herein include the hinge region present in extracellular regions of type 1 membrane proteins, such as CD8a, CD4, CD28, and CD7, which may be wild-type or variants thereof. In particular embodiments, a hinge includes a CD8a hinge set forth in SEQ ID NO: 129.

In particular embodiments, a spacer region includes a hinge region of a type II C-lectin interdomain (stalk) region or a cluster of differentiation (CD) molecule stalk region. A “stalk region” of a type II C-lectin or CD molecule refers to the portion of the extracellular domain of the type II C-lectin or CD molecule that is located between the C-type lectin-like domain (CTLD; e.g., similar to CTLD of natural killer cell receptors) and the hydrophobic portion (transmembrane domain). For example, the extracellular domain of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids between the stalk region and the transmembrane region or the CTLD. In another example, the 233 amino acid human NKG2A protein (UniProt ID P26715.1) has a hydrophobic portion (transmembrane domain) ranging from amino acids 71-93 and an extracellular domain ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

(iv-e) Tags and Selectable Markers. Tags and selectable markers that can optionally be used within CAR are described elsewhere herein.

(v) Cells Genetically Modified to Express Activity-Inducible Fusion Proteins. The present disclosure includes cells genetically modified to express an activity-inducible fusion protein. As used herein, the term “genetically modified” or “genetically engineered” refers to the addition of extra genetic material in the form of DNA or RNA into the cell. The terms “genetically modified cells” and “modified cells” are used interchangeably. In particular embodiments, a cell genetically modified to express an activity-inducible fusion protein includes an immune effector cell. An “immune effector cell” includes any cell of the immune system that has one or more effector functions (e.g., cytotoxic cell killing activity, secretion of cytokines, induction of antibody-dependent cell cytotoxicity (ADCC) and/or complement-dependent cytotoxicity (CDC). Immune effector cells are a subtype of immune cells.

Immune cells of the disclosure can be autologous/autogeneic (“self”) or non-autologous (“non-self,” e.g., allogeneic, syngeneic or xenogeneic). “Autologous” refers to cells from the same subject. “Allogeneic” refers to cells of the same species that differ genetically to a cell in comparison. “Syngeneic” refers to cells of a different subject that are genetically identical to the cell in comparison. “Xenogeneic” refers to cells of a different species to the cell in comparison. In particular embodiments, modified cells of the disclosure are autologous or allogeneic.

In particular embodiments, genetically modified cells include lymphocytes. In particular embodiments, genetically modified cells include T cells, B cells, natural killer (NK) cells, monocytes/macrophages, or HSPC.

Most T cells have a T-cell receptor (TCR) composed of two separate peptide chains (the α- and β-TCR chains). γd T cells represent a small subset of T cells that possess a distinct T cell receptor (TCR) made up of one γ-chain and one d-chain.

CD3 is expressed on all mature T cells. T cells can further be classified into cytotoxic T cells (CD8+ T cells, also referred to as CTLs) and helper T cells (CD4+ T cells).

Cytotoxic T cells destroy virally infected cells and tumor cells and are also implicated in transplant rejection. These cells recognize their targets by binding to antigen associated with MHC class I, which is present on the surface of nearly every cell of the body.

Central memory T cells (TCM) refer to antigen experienced CTL that express CD62L or CCR7 and CD45RO and does not express or has decreased expression of CD45RA as compared to naive cells.

Effector memory T cells (TEM) refer to an antigen experienced T-cell that does not express or has decreased expression of CD62L as compared to central memory cells and does not express or has decreased expression of CD45RA as compared to a naive cell. In particular embodiments, effector memory T cells are negative for expression of CD62L and CCR7, compared to naive cells or central memory cells, and have variable expression of CD28 and CD45RA. Effector T cells are positive for granzyme B and perforin as compared to memory or naive T cells.

Helper T cells assist other immune cells such as activating of cytotoxic T cells and macrophages and facilitating the maturation of B cells, among other functions. Helper T cells become activated when they are presented with peptide antigens by MHC class II molecules that are expressed on the surface of antigen presenting cells (APCs). Once activated, they divide rapidly and secrete cytokines that regulate or assist in the active immune response.

Natural killer T (NKT) cells are a subset of T cells that co-express an αβ T-cell receptor, but also express a variety of molecular markers that are typically associated with natural killer cells, such as NK1.1 (CD161), CD16, and/or CD56.

Natural killer cells (also known as K cells and killer cells) express CD8, CD16 and CD56 but do not express CD3. NK cells also express activating receptors such as NKp46 and inhibitory receptors such as NKG2A that regulate NK cell cytotoxic function against tumor and virally infected cells.

Tumor-infiltrating lymphocytes (TILs) refers to immune cells that have moved from the blood into a tumor and can function to recognize and kill cancer cells. Marrow-infiltrating lymphocytes (MILs) are antigen-experienced immune cells that travel to and remain in the bone marrow. Mucosal-associated invariant T (MAIT) cells are innate-like T cells which are found in the mucosa, blood, and secondary lymphoid organs (SLO), and display effector phenotype. MAIT cells display a semi-invariant T cell receptor (TCR) and are restricted by the major histocompatibility complex related molecule, MR1.

Macrophages (and their precursors, monocytes) reside in every tissue of the body where they engulf apoptotic cells, pathogens and other non-self-components. Monocytes/macrophages express CD11b, F4/80, CD68, CD11c, IL-4Ra, and/or CD163.

Immature dendritic cells (i.e., pre-activation) engulf antigens and other non-self-components in the periphery and subsequently, in activated form, migrate to T cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic stem cells (HSC) refer to undifferentiated hematopoietic cells that are capable of self-renewal and differentiation into all other hematopoietic cell types. HSC are CD34+.

Hematopoietic progenitor cells (HPC) are derived from HSC and are capable of further differentiation into mature cell types. HPC can self-renew or can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T cells, B cells, and NK cells. HPC are CD24^{lo} Lin⁻CD117⁺.

HSPC refer to a cell population having HSC and HPC. HSPC cell populations can be positive for CD34, CD43, CD45RO, CD45RA, CD59, CD90, CD109, CD117, CD133, CD166, HLA DR, or a combination thereof.

Induced pluripotent stem cells (iPSCs) refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing or contacting with reprogramming factors.

(vi) Methods to Modify Cells Ex Vivo and In Vivo. The present disclosure provides methods for collecting, enriching for, culturing, and modifying cells to express an activity-inducible fusion protein ex vivo and/or genetically modifying immune cells in vivo utilizing cell-targeted delivery methods.

In particular embodiments, lymphocytes are isolated from a sample such as blood or a blood-derived sample, an apheresis or a leukapheresis product. Exemplary samples include whole blood, peripheral blood mononuclear cells (PBMCs), bone marrow, thymus, cancer tissue, lymphoid tissue, spleen, or other appropriate sources.

Sources of HSPC include, for example, peripheral blood (see U.S. Pat. Nos. 5,004,681; 7,399,633; and U.S. Pat. No. 7,147,626; Craddock, et al., 1997, Blood 90 (12): 4779-4788; Jin, et al., 2008, Journal of Translational Medicine 6:39; Pelus, 2008, Curr. Opin. Hematol. 15 (4): 285-292; Papayannopoulou, et al., 1998, Blood 91 (7): 2231-2239; Tricot, et al., 2008, Haematologica 93 (11): 1739-1742; and Weaver et al., 2001, Bone Marrow Transplantation 27 (2): S23-S29).

Methods regarding collection, anti-coagulation and processing, etc. of blood samples can be found in, for example, Alsever, et al., 1941, N.Y. St. J. Med. 41:126; De Gowin, et al., 1940, J. Am. Med. Ass. 114:850; Smith, et al., 1959, J. Thorac. Cardiovasc. Surg. 38:573; Rous and Turner, 1916, J. Exp. Med. 23:219; and Hum, 1968, Storage of Blood, Academic Press, New York, pp. 26-160.

In particular embodiments, collected cells are washed, centrifuged, and/or incubated in the presence of one or more reagents, for example, to remove unwanted components, enrich for desired components, lyse or remove cells sensitive to particular reagents. The isolation can include one or more of various cell preparation and separation steps, including separation based on one or more properties, such as size, density, sensitivity or resistance to particular reagents, and/or affinity, e.g., immunoaffinity, to antibodies or other binding partners.

In particular embodiments, one or more of the cell populations enriched, isolated and/or selected from a sample by the provided methods are cells that are positive for (marker+) or express high levels (marker^{hi}) of one or more particular markers, such as surface markers, or that are negative for (marker-) or express relatively low levels (marker^{lo}) of one or more markers.

In particular embodiments, T cells can be isolated from peripheral blood mononuclear cells (PBMCs) by lysing the red blood cells and depleting the monocytes, for example, by centrifugation through a PERCOLL™ gradient. In particular embodiments, a specific subpopulation of T cells, expressing CD3, CD28, CD4, CD8, CD45RA, and CD45RO is further isolated by positive or negative selection techniques. In particular embodiments, cell sorting and/or selection occurs via negative magnetic immunoadherence or flow cytometry using a cocktail of monoclonal antibodies directed to cell surface markers present on the cells negatively selected. For example, to enrich for CD4⁺ cells by negative selection, a monoclonal antibody cocktail that typically includes antibodies to CD14, CD20, CD11b, CD16, HLA-DR, and CD8 can be used.

Following isolation and/or enrichment, cells can be expanded to increase the number of cells. In particular embodiments, T cells can be activated and expanded before or after genetic modification to express an activity-inducible fusion protein, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US 2006/0121005.

Generally, the T cells are expanded by contact with a surface having attached thereto an agent that stimulates a CD3 TCR complex associated signal and a ligand that stimulates a co-stimulatory molecule on the surface of the T cells. In particular embodiments, PBMCs or isolated T cells are contacted with a stimulatory agent and costimulatory agent, such as anti-CD3 and anti-CD28 antibodies, generally attached to a bead or other surface, in a culture medium with appropriate cytokines (see Berg et al., Transplant Proc. 30 (8): 3975-3977, 1998; Haanen et al., J. Exp. Med. 190 (9): 13191328, 1999; Garland et al., J. Immunol Meth. 227 (1-2): 53-63, 1999). In particular embodiments, the T cells may be activated and stimulated to proliferate with feeder cells and appropriate antibodies and cytokines using methods such as those described in U.S. Pat. Nos. 6,040,177; 5,827,642; and WO 2012/129514.

In particular embodiments, artificial APC (aAPC) can be made by engineering K562, U937, 721.221, T2, and C1R cells to direct the stable expression and secretion of a variety of co-stimulatory molecules and cytokines. aAPCs are described in WO 03/057171 and US 2003/0147869.

In particular embodiments, HSPCs can be isolated and/or expanded following methods described in, for example, U.S. Pat. Nos. 7,399,633; 5,004,681; US 2010/0183564; WO2006/047569; WO2007/095594; WO 2011/127470; and WO 2011/127472; Vamum-Finney, et al., 1993, Blood 101:1784-1789; Delaney, et al., 2005, Blood 106:2693-2699; Ohishi, et al., 2002, J. Clin. Invest. 110:1165-1174; Delaney, et al., 2010, Nature Med. 16 (2): 232-236; and Chapter 2 of Regenerative Medicine, Department of Health and Human Services, August 2006, and the references cited therein. The collection and processing of other cell types described herein are known by one of ordinary skill in the art.

In particular embodiments, the isolating, incubating, expansion, and/or engineering steps are carried out in a sterile or contained environment and/or in an automated fashion, such as controlled by a computer attached to a device in which the steps are performed. Final formulation of modified cells into modified formulations for administration is described elsewhere herein.

Targeted viral vectors and/or nanoparticles can also be used to genetically-modify immune cells in vivo. Viral vectors that can be used to deliver fusion protein-encoding genes to cells are described elsewhere herein, and numerous targeted (e.g., pseudotyped) viral vectors are known in the art.

Exemplary cell-targeted nanoparticles include a cell targeting ligand (e.g., CD3, CD4, CD8, CD34) on the surface of the nanoparticle wherein the cell targeting ligand results in selective uptake of the nanoparticle by a selected cell type. The nanoparticle then delivers gene modifying components that result in expression of the activity-inducible fusion protein.

Exemplary nanoparticles include liposomes (microscopic vesicles including at least one concentric lipid bilayer surrounding an aqueous core), liposomal nanoparticles (a liposome structure used to encapsulate another smaller nanoparticle within its core); and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum and silica).

For in vivo delivery and cellular uptake, nanoparticles can have a neutral or negatively-charged coating and a size of 130 nm or less. Dimensions of the nanoparticles can be determined using, e.g., conventional techniques, such as dynamic light scattering and/or electron microscopy.

(vii) Production of Activity-Inducible Fusion Proteins. An activity-inducible fusion protein according to the present disclosure can be produced by any methods known in the art. This discussion applies equally to CAR, when CAR are used as an aspect of the disclosure. In particular embodiments, an activity-inducible fusion protein is produced using recombinant DNA techniques. A nucleic acid encoding the several regions of the activity-inducible fusion protein can be prepared and assembled into a complete coding sequence by standard techniques of molecular cloning. The resulting coding regions can be inserted into an expression vector and used to transform a cell or cell line.

The term “gene” refers to a nucleic acid sequence (used interchangeably with polynucleotide or nucleotide sequence) that encodes an activity-inducible fusion protein, components of an activity-inducible fusion protein, or a molecule co-expressed with an activity-inducible fusion protein as described herein. This definition includes various sequence polymorphisms, mutations, and/or variants wherein such alterations do not substantially affect the function of the encoded protein. The term “gene” may include not only coding sequences but also regulatory regions such as promoters, enhancers, and termination regions. Gene sequences encoding a molecule can be DNA or RNA that directs the expression of the activity-inducible fusion protein. These nucleic acid sequences may be a DNA strand sequence that is transcribed into RNA or an RNA sequence that is translated into protein.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids. Thus, a gene codes for a protein if transcription and translation of mRNA corresponding to that gene produces the protein in a cell or other biological system. A “gene encoding a protein” includes all nucleotide sequences that are degenerate versions of each other and that code for the same amino acid sequence or amino acid sequences of substantially similar form and function.

Polynucleotide gene sequences encoding more than one portion of an expressed activity-inducible fusion protein can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

In an exemplary nucleic acid construct employed in the present disclosure, the promoter is operably linked to the nucleic acid sequence encoding an activity-inducible fusion protein, i.e., they are positioned so as to promote transcription of mRNA from the DNA encoding the activity-inducible fusion protein. The promoter can be of genomic origin or synthetically generated. The promoters may or may not be associated with enhancers, wherein the enhancers may be naturally associated with the particular promoter or associated with a different promoter. A variety of promoters for use in cells are well-known in the art (e.g., a CD4 promoter). The promoter can be constitutive or inducible, where induction is associated with a specific cell type or a specific stage of development, for example. Alternatively, a number of well-known viral promoters are also suitable. Promoters of interest include: a viral simian virus 40 (SV40) (e.g., early or late) promoter; a Moloney murine leukemia virus (MoMLV) long terminal repeat (LTR) promoter; a Rous sarcoma virus (RSV) LTR promoter; a herpes simplex virus (HSV) (thymidine kinase) promoter; a glyceraldehyde 3-phosphate dehydrogenase (GAPDH) promoter; heat shock protein 70 kDa (HSP70) promoter; a Ubiquitin C (UBC) promoter; or a phosphoglycerate kinase-1 (PGK) promoter.

In certain embodiments, a signal sequence directing a CAR to the surface membrane can be used and can include an endogenous signal sequence of the N-terminal component of the CAR. Optionally, in some instances, it may be desirable to exchange this sequence for a different signal sequence. However, the signal sequence selected should be compatible with the secretory pathway of the CAR-expressing cells so that the CAR is presented on the surface of its expressing cell. Particular embodiments disclosed herein utilize a GM-CSF signal peptide or a CD8 signal peptide.

Similarly, a termination region may be provided by the naturally occurring or endogenous transcriptional termination region of the nucleic acid sequence encoding the C-terminal component of the activity-inducible fusion protein. Alternatively, the termination region may be derived from a different source. For the most part, the source of the termination region is generally not considered to be critical to the expression of a recombinant protein and a wide variety of termination regions can be employed without adversely affecting expression.

As will be appreciated by one of skill in the art, in some instances, a few amino acids at the ends of an activity-inducible fusion protein can be deleted, usually not more than 10, more usually not more than 5 residues, for example. Also, it may be desirable to introduce a small number of amino acids at the borders, usually not more than 10, more usually not more than 5 residues. The deletion or insertion of amino acids may be as a result of the needs of the construction, providing for convenient restriction sites, ease of manipulation, improvement in levels of expression, or the like. In addition, the substitute of one or more amino acids with a different amino acid can occur for similar reasons.

In any of the embodiments described herein, a polynucleotide can include a sequence that encodes a self-cleaving polypeptide between the polynucleotide segment encoding the activity-inducible fusion protein or CAR and a polynucleotide encoding a selection (e.g., transduction) marker (e.g., EGFRt, Her2tG, CD19t, or DHFRdm). Exemplary nucleic acid sequences encoding 2A peptides are set forth in, for example, Kim et al. (PLOS One 6: e18556 (2011)) and Donnelly et al. (J. Gen. Virol. 82:1027-1041 (2001)).

Desired genes encoding activity-inducible fusion proteins can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, mammalian artificial chromosomes (Vos, 1998, Curr. Op. Genet. Dev. 8:351-359), liposomes (Tarahovsky and Ivanitsky, 1998, Biochemistry (Mosc) 63:607-618), ribozymes (Branch and Klotman, 1998, Exp. Nephrol. 6:78-83), triplex DNA (Chan and Glazer, 1997, J. Mol. Med. 75:267-282), etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressed by the cell and, in certain instances, preferably heritable and expressed in its cell progeny.

In particular embodiments, a gene encoding an activity-inducible fusion protein can be introduced into cells in a vector. A “vector” is a nucleic acid molecule that is capable of transporting another nucleic acid. Vectors may be, e.g., plasmids, cosmids, viruses, or phage. An “expression vector” is a vector that is capable of directing the expression of a protein encoded by one or more genes carried by the vector when it is present in the appropriate environment.

Viral vectors can be derived from numerous viruses. “Lentivirus” refers to a genus of retroviruses that are capable of infecting dividing and non-dividing cells and typically produce high viral titers. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

Additional examples of viral vectors include those derived from foamy viruses, adenoviruses (e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), adeno-associated virus (AAV; see, e.g., U.S. Pat. No. 5,604,090; Kay et al., 2000; Nakai et al., z1998), alphaviruses, cytomegaloviruses (CMV), flaviviruses, herpes viruses (e.g., herpes simplex), influenza viruses, papilloma viruses (e.g., human and bovine papilloma virus; see, e.g., U.S. Pat. No. 5,719,054), poxviruses, vaccinia viruses, etc. See Kozarsky and Wilson, 1993; Rosenfeld, et al., 1991; Rosenfeld, et al., 1992; Mastrangeli, et al., 1993; Walsh, et al., 1993; and Lundstrom, 1999. Examples include modified vaccinia Ankara (MVA) and NYVAC, or strains derived therefrom. Other examples include avipox vectors, such as a fowlpox vectors (e.g., FP9) or canarypox vectors (e.g., ALVAC and strains derived therefrom). For additional information regarding viral vectors for gene delivery, see Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19:673-686; Miller, et al., 1993, Meth. Enzymol. 217:581-599); Naldini et al. (1996) Science 272 (5259): 263-267; Naldini et al. (1996) Proceedings of the National Academy of Sciences 93 (21): 11382-11388; Zufferey et al. (1997) Nature biotechnology 15 (9): 871-875; Dull et al. (1998) Journal of virology 72 (11): 8463-8471; U.S. Pat. Nos. 6,013,516; and 5,994,136).

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9 (8): 805-7; Ramirez et al., Nucl Acids Res, 2012, 40 (12): 5560-8; Kim et al., Genome Res, 2012, 22 (7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14 (l): 49-55; Beurdeley et al., Nat Commun, 2013, 4:1762; Scharenberg et al., Curr Gene Ther, 2013, 13 (4): 291-303; Gaj et al., Nat Methods, 2012, 9 (8): 805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Cells that have been successfully genetically modified to express an activity-inducible fusion protein ex vivo can be sorted based on, for example, expression of a transduction marker, and further processed.

(viii) Modified Formulations, Modifying Formulations, and Drug Compositions. Formulations described herein can include ex vivo genetically modified cells (i.e., modified formulations) or can include viral vectors or nanoparticles that result in in vivo genetic modification of cells to express an activity-inducible fusion protein (modifying formulations). As used herein, compositions include a drug molecule that binds an hsp90 binding domain present on an expressed activity-inducible fusion protein and/or results in a conformation change of the activity-inducible fusion protein.

A “pharmaceutical” formulation or composition includes an active compound for administration (e.g., a genetically modified cell, viral vector, nanoparticle, or drug molecule) within a pharmaceutically-acceptable carrier.

The phrase “pharmaceutically acceptable” refer to those compounds, materials, and/or dosage forms which are, within the scope of sound medical judgment, suitable for use in contact with the tissues of human beings and animals without excessive toxicity, commensurate with a reasonable benefit/risk ratio. In certain instances, pharmaceutically-acceptable carriers have been approved by a relevant regulatory agency (e.g., the United States Food and Drug Administration (US FDA)).

Depending on the context and active compound for delivery, “pharmaceutically acceptable carriers” includes any adjuvant, excipient, glidant, diluent, preservative, dye/colorant, surfactant, wetting agent, dispersing agent, suspending agent, stabilizer, isotonic agent, solvent, surfactant, or emulsifier which meets the requirements noted above. Exemplary pharmaceutically acceptable carriers are disclosed in Remington's Pharmaceutical Sciences, 18th Ed. Mack Printing Company, 1990. Moreover, formulations and compositions can be prepared to meet sterility, pyrogenicity, general safety, and purity standards as required by the US FDA Office of Biological Standards and/or other relevant foreign regulatory agencies.

Exemplary pharmaceutically-acceptable carriers include saline, buffered saline, physiological saline, water, Hanks' solution, Ringer's solution, Nonnosol-R (Abbott Labs), PLASMA-LYTE AR (Baxter Laboratories, Inc., Morton Grove, IL), glycerol, ethanol, and combinations thereof. In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols, amino acids, organic sugars or sugar alcohols, PEG, sulfur-containing reducing agents, bovine serum albumin, gelatin or immunoglobulins, polyvinylpyrrolidone, and saccharides.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens, catechol, resorcinol, cyclohexanol, and 3-pentanol.

Therapeutically effective amounts of cells within modified formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In modified formulations disclosed herein, cells are generally in a volume of a liter or less, 500 ml or less, 250 ml or less, or 100 ml or less. Hence the density of administered cells is typically greater than 10⁴ cells/ml, 10⁷ cells/ml, or 108 cells/ml.

Therapeutically effective amounts of active ingredients (vectors, nanoparticles) within modifying formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts of drug molecules within compositions can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

In particular embodiments, modified formulations can include one or more genetically modified cell type (e.g., modified T cells, NK cells, or stem cells) or genetically modified cells that express one or more activity-inducible fusion protein types. The different populations of genetically modified cells can be provided in different ratios. Further, modifying formulations can deliver nucleic acids resulting in the genetic modification of more than one cell type and/or the expression of different activity-inducible fusion proteins.

Certain modified formulations include immune cells that express more than one activity-inducible fusion protein type. For example, an immune cell (e.g., a T cell) can be modified to express different transcription factors wherein the different transcription factors initiate transcription of different genes. In this scenario, the activity-inducible fusion proteins having transcription factors can also include different hsp90 binding domains, so that their activation states can be individually controlled by administration of different drug molecules (e.g., small molecule estrogen analogs).

Certain formulations can result in the expression of multiple activity-inducible fusion proteins, wherein the activity-inducible fusion proteins can be individually activated or inactivated through inclusion of different EBD. The different EBD can include EBD (E353A), activatable by the administration of ES8, EBD (L384M, M421G, G521R), activatable by the administration of CMP8, and EBD ERT2, activatable by the administration of 4-OHT. Numerous additional combinations are possible, based on the content of the current disclosure.

Exemplary combinations of fusion proteins expressing different transcription factors and different hsp90 binding domains include (i) ca-STAT3 and ER (T2) with caSTAT5a and EBD (L384M, M421G, G521R); (ii) caSTAT5a and ER (T2) with TCF7 and EBD (L384M, M421G, G521R); (iii) TCF7 and ER (T2) with c-Myc and EBD (L384M, M421G, G521R); or (iv) caSTAB5b and ER (T2) with TCF7 and EBD (L384M, M421G, G521R). This approach of utilizing different transcription factors with different hsp90 binding domains is referred to herein as a transcription factor combination therapy.

Use of systems where combinations of multiple transcription factors fused with varying estrogen receptors can be used. This can allow for the modular activation of one of the transcription factors upon introducing the respective estrogen analog, while maintaining the other(s) at an inactive state until the introduction of their activating estrogen analog. If the transcription factors utilized in the systems have different functions, or subsequent effects on cell behavior, this may allow for a more efficient and effective therapeutic response. A particular embodiment includes caSTAT5 fused to ER (T2) and caSTAT3 fused with EBD (CMP8)—or vice versa—in CAR T cell systems. caSTAT3 has been reported to inhibit proliferation of T cells, whereas caSTAT5 is pro-proliferative, however both have shown they increase T cell survival. In the example provided above, 4-OHT may be introduced when the T cell faces a tumor challenge, thus activating caSTAT5, and inducing pro-proliferative effects. Alternatively, CMP8 may be introduced after the previously described event, activating caSTAT3, and helping the T cell to recover from the challenge while discouraging exhaustion.

In certain examples, formulations result in the expression of an activity-inducible fusion protein and a co-stimulatory immune molecule (e.g., CD28, 4-1BB, OX40, ICOS) wherein the activity-inducible fusion protein and the co-stimulatory immune molecule each include a different hsp90 binding domain. In certain examples, formulations result in the expression of an activity-inducible fusion protein and two different co-stimulatory immune molecules (e.g., CD28, 4-1BB, OX40, ICOS) wherein the activity-inducible fusion protein and the different co-stimulatory immune molecules each include a different hsp90 binding domain. In certain examples, formulations result in the expression of two activity-inducible fusion protein types and two different co-stimulatory immune molecules (e.g., CD28, 4-1BB, OX40, ICOS) wherein the two activity-inducible fusion protein types have different transcription factors and different hsp90 binding domains while the different co-stimulatory immune molecules each include the same hsp90 binding domain. In certain examples, formulations result in the expression of two activity-inducible fusion protein types and two different co-stimulatory immune molecules (e.g., CD28, 4-1BB, OX40, ICOS) wherein the two activity-inducible fusion protein types have different transcription factors and different hsp90 binding domains and the different co-stimulatory immune molecules each include a different hsp90 binding domain. In this example, the hsp90 binding domains of the co-stimulatory molecules can match that of a transcription factor or be distinct from the hsp90 binding domains of the transcription factor.

Modified formulations can also include different immune cells expressing different activity-inducible fusion proteins. For example, certain individually modified immune cells express only one type of activity-inducible fusion protein but are formulated with immune cells modified to express different types of activity-inducible fusion protein (e.g., different transcription factors associated with different EBD/drug molecule combinations). The immune cells can be of the same type (all T cells) or can include a mixture of different types (e.g., T cells, NK cells, and/or HSPC).

Modifying formulations can also be prepared to lead to in vivo populations of immune cells having these characteristics (e.g., expression of different activity-inducible fusion protein types by a single immune cell; expression of a different activity-inducible fusion protein types by different immune cells; expression of a same activity-inducible fusion protein type by different types of immune cells; and/or expression of different activity-inducible fusion protein types by different types of immune cells; inclusion of activity-inducible co-stimulatory or inhibitory molecules).

Formulations and compositions can be prepared for administration by, e.g., injection, infusion, perfusion, lavage, or ingestion. The formulations and compositions can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

In some instances, it can be useful to cryopreserve modified cell formulations of the disclosure. As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to ameliorate or prevent cell damage due to freezing at low temperatures or warming to room temperature. Cryoprotective agents and optimal cooling rates can protect against cell injury. Cryoprotective agents which can be used include dimethyl sulfoxide (DMSO) (Lovelock and Bishop, Nature, 1959; 183:1394-1395; Ashwood-Smith, Nature, 1961; 190:1204-1205), glycerol, polyvinylpyrrolidine (Rinfret, Ann. N.Y. Acad. Sci., 1960; 85:576), and polyethylene glycol (Sloviter and Ravdin, Nature, 1962; 196:48). In particular embodiments, the cooling rate is 1° to 3° C./minute. After at least two hours, the cells reach a temperature of −80° C. and can be placed directly into liquid nitrogen (−196° C.) for permanent storage such as in a long-term cryogenic storage vessel.

(ix) Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with (i) modified formulations and/or modifying formulations, and (ii) drug compositions disclosed herein. Treating subjects includes delivering therapeutically effective amounts. Therapeutically effective amounts include those that provide effective amounts, prophylactic treatments and/or therapeutic treatments without undue toxicity.

An “effective amount” is the amount of a formulation or composition necessary to result in a desired physiological effect. Effective amounts are often administered for research purposes. Effective amounts disclosed herein can cause chromium or cytokine release in an assay of cell activation.

A “prophylactic treatment” includes a treatment administered to a subject who does not display signs or symptoms of a condition (e.g., cancer or an infection) or displays only early signs or symptoms of the condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment or therapeutic treatment are not mutually exclusive, and in particular embodiments, administered dosages may accomplish more than one treatment type.

Therapeutically effective amounts can be achieved by administering single or multiple doses during the course of a treatment regimen (e.g., daily, every other day, every 3 days, weekly, every 2 weeks, monthly, every 2 months, every 4 months, every 6 months, yearly, etc.).

As indicated, the formulations and compositions can be administered by injection, transfusion, implantation or transplantation. Modifying formulations and drug compositions can also be administered orally or via inhalation. In particular embodiments, formulations and compositions are administered parenterally. The phrases “parenteral administration” and “administered parenterally” refer to modes of administration other than enteral and topical administration, usually by injection, and includes, intravascular, intravenous, intramuscular, intraarterial, intrathecal, intracapsular, intratumoral, intraperitoneal, and subcutaneous, injection and infusion. In particular embodiments, the formulations and compositions described herein are administered to a subject by direct injection into a tumor, lymph node, or site of disease. In particular embodiments, drug compositions are administered orally.

In certain examples the disclosure provides methods of performing cellular immunotherapy in a subject having a disease or disorder including: administering a modified or modifying formulation that results in in vivo expression of an activity-inducible fusion protein whose activation state is constitutively “OFF”. In these examples, the methods further include administering a drug composition that allows activation of the activity-inducible fusion protein upon administration of the drug composition. In some alternatives, the drug composition is delivered prior to, at the same time as the modified or modifying formulation, or at later time points after the modified or modifying formulation has been administered.

In some alternatives, the drug composition is administered with the modified or modifying formulation, and if a toxic effect of the formulation is observed the drug composition is withdrawn until the toxic effects diminish. After the symptoms of toxicity diminish, the drug composition can be administered again.

In certain examples the disclosure provides methods of performing cellular immunotherapy in a subject having a disease or disorder including: administering a modified or modifying formulation that results in in vivo expression of at least two types of activity-inducible fusion proteins whose activation state is constitutively “OFF”. The two types of activity-inducible fusion proteins include different transcription factors and have different hsp90 binding domains that bind different drug molecules. In these examples, the methods further include selectively administering one or more of the different drug molecule compositions to selectively allow activation of different activity-inducible fusion proteins upon drug administration. In some alternatives, one or more of the drug compositions are delivered prior to, at the same time as the modified or modifying formulation, or at later time points after the modified or modifying formulation has been administered.

In certain examples the disclosure provides methods of performing cellular immunotherapy in a subject having a disease or disorder including: administering a modified or modifying formulation that results in in vivo expression of at least one activity-inducible fusion protein whose activation state is constitutively “OFF” and at least one co-stimulatory molecule whose activation state is constitutively “OFF”. The activity-inducible fusion protein and the co-stimulatory molecule have different hsp90 binding domains that bind different drug molecules. In these examples, the methods further include selectively administering one or more of the different drug molecule compositions to selectively allow activation of the activity-inducible fusion protein upon drug administration and/or the co-stimulatory molecule. In some alternatives, one or more of the drug compositions are delivered prior to, at the same time as the modified or modifying formulation, or at later time points after the modified or modifying formulation has been administered.

In particular embodiments, combinations of multiple transcription factors fused with varying estrogen receptors can be administered to a subject. This can allow for the modular activation of one of the transcription factors upon introducing the respective estrogen analog, while maintaining the other(s) at an inactive state until the introduction of their activating estrogen analog. If the transcription factors utilized in the systems have different functions, or subsequent effects on cell behavior, this may allow for a more efficient and effective therapeutic response. A particular embodiment includes administering caSTAT5 fused to ER (T2) and caSTAT3 fused with EBD (CMP8)—or vice versa—in CAR T cell systems. caSTAT3 has been reported to inhibit proliferation of T cells, whereas caSTAT5 is pro-proliferative, however both have shown they increase T cell survival. As described previously, 4-OHT may be administered when the T cell faces a tumor challenge, thus activating caSTAT5, and inducing pro-proliferative effects. Alternatively, CMP8 may be administered after the previously described event, activating caSTAT3, and helping the T cell to recover from the challenge while discouraging exhaustion.

In some alternatives, one or more drug compositions are administered with the modified or modifying formulation, and if a toxic effect of the formulation is observed one or more of the drug compositions is withdrawn until the toxic effects diminish. After the symptoms of toxicity diminish, one or more drug compositions can be administered again. Toxicity can be observed based on, for example, levels of TNFα or IFNγ that exceed a clinically-relevant threshold.

In some alternatives, the drug composition(s) is administered with the modified or modifying formulation but once the subject has a decrease in cancer cells or virally-infected cells, the drug composition is not administered for a period of time to allow the modified cells to rest. Administration of the drug composition can also be stopped when a cancer is in remission or an infection has been cleared.

Cancers that can be treated by modified or modifying formulations and drug compositions disclosed herein include: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B cell lymphoma, T cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma. Other exemplary cancers that can be treated according to the disclosure include hematopoietic tumors of lymphoid lineage, for example T cell and B cell tumors, including: T cell disorders such as T-prolymphocytic leukemia (T-PLL), including of the small cell and cerebriform cell type; large granular lymphocyte leukemia (LGL) of the T cell type; Sezary syndrome (SS); adult T cell leukemia lymphoma (ATLL); hepatosplenic T cell lymphoma; peripheral/post-thymic T cell lymphoma (pleomorphic and immunoblastic subtypes); angioimmunoblastic T cell lymphoma; angiocentric (nasal) T cell lymphoma; anaplastic (Ki 1+) large cell lymphoma; intestinal T cell lymphoma; and T-lymphoblastic lymphoma/leukemia (T-Lbly/T-ALL).

Optional CAR with ligand binding domains that bind the following exemplary cancer antigens can be selected based on the cancer experienced by a subject: bladder cancer antigens: MUC16, PD-L1, EGFR; breast cancer antigens: HER2, ERBB2, ROR1, PD-L1, EGFR, MUC16, FOLR, CEA; cholangiocarcinoma antigens: mesothelin, PD-L1, EGFR; colorectal cancer antigens: CEA, PD-L1, EGFR; glioblastoma antigens: EGFR variant III (EGFRvlll), IL13Ra2; lung cancer antigens: ROR1, PD-L1, EGFR, mesothelin, MUC16, FOLR, CEA, CD56; Merkel cell carcinoma antigens: CD56, PD-L1, EGFR; mesothelioma antigens: mesothelin, PD-L1, EGFR; neuroblastoma antigens: ROR1, glypican-2, CD56, disialoganglioside, PD-L1, EGFR; ovarian cancer antigens: EpCam, L1-CAM, MUC16, folate receptor (FOLR), Lewis Y, ROR1, mesothelin, WT-1, PD-L1, EGFR, CD56; melanoma antigens: Tyrosinase related protein 1 (TYRP1/gp75); GD2, PD-L1, EGFR; multiple myeloma antigens: B-cell maturation antigen (BCMA), PD-L1, EGFR; pancreatic cancer antigens: mesothelin, CEA, CD24, ROR1, PD-L1, EGFR, MUC16; prostate cancer antigens: PSMA, WT1, Prostate Stem Cell antigen (PSCA), SV40 T, PD-L1, EGFR; renal cell carcinoma antigens: carboxy-anhydrase-IX (CAIX); PD-L1, EGFR; and stem cell cancer antigens: CD133, PD-L1, EGFR. Other examples are known to those of ordinary skill in the art. These groupings can be utilized to create combination therapies.

Particular CAR combination therapies include CAR with binding domains that bind (i) CD19, CD22, and/or BAFF-R (e.g., CD19 and CD22) for the treatment of acute lymphoblastic leukemia (ALL); (ii) Her2, B7H3, EGFR, and/or IL13Ra2 for the treatment of brain tumors; and (iii) CD33 and CD123 for the treatment of acute myeloid leukemia (AML).

In certain examples, a cancerous sample from a subject can be characterized for the presence of certain biomarkers or cell surface markers. For example, breast cancer cells from a subject can be positive or negative for each of Her2Neu, Estrogen receptor, and/or the Progesterone receptor. A tumor antigen or cell surface molecule that is found on the individual subject's tumor cells as well as a CAR with a binding domain that binds the antigen is selected. Combinations may also be selected to create a CAR combination therapy.

In particular embodiments, therapeutically effective amounts of formulations and drug compositions provide anti-cancer effects. Anti-cancer effects include a decrease in the number of malignant cells, decrease in the number of metastases, a decrease in tumor volume, an increase in life expectancy, induced chemo- or radio-sensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

Infections that can be treated by disclosed formulations and compositions include bacterial, viral, fungal, parasitic, and arthropod infections. In particular embodiments, the infections are chronic. In particular embodiments, bacterial infections can include infections caused by Staphylococcus spp., Streptococcus spp., Campylobacter jejuni, Clostridium botulinum, Clostridium difficile, Escherichia coli, Listeria monocytogenes, Salmonella, Vibrio, Chlamydia trachomatis, Neisseria gonorrhoeae, and Treponema pallidum. In particular embodiments, viral infections can include infections caused by rhinovirus, influenza virus, respiratory syncytial virus (RSV), coronavirus (e.g., MERS, SARS, SARS-COV-2), herpes simplex virus-1 (HSV-1), varicella-zoster virus (VZV), hepatitis A, norovirus, rotavirus, human papillomavirus (HPV), hepatitis B, human immunodeficiency virus (HIV), herpes simplex virus-2 (HSV-2), Epstein-Barr virus (EBV), West Nile virus (WNV), enterovirus, hepatitis C, human T-lymphotrophic virus-1 (HTLV-1), and Merkel cell polyomavirus (MCV). In particular embodiments, fungal infections can include infections caused by Trychophyton spp. and Candida spp. In particular embodiments, parasitic infections can include infections caused by Giardia, toxoplasmosis, E. vermicularis, Trypanosoma cruzi, Echinococcosis, Cysticercosis, Toxocariasis, Trichomoniasis, and Amebiasis. In particular embodiments, arthropod infections can include infections spread by arthropods infected with viruses or bacteria, including California encephalitis, Chikungunya, dengue, Eastern equine encephalitis, Powassan, St. Louis encephalitis, West Nile, Yellow Fever, Zika, Lyme disease, and babesiosis.

In particular embodiments, therapeutically effective amounts of formulations and drug compositions provide anti-infection effects. Anti-infection effects include a decrease in: the amount or level of infective pathogen, fatigue, loss of appetite, weight loss, fevers, night sweats, chills, aches and pains, diarrhea, bloating, abdominal pain, skin rashes, coughing, and/or a runny nose.

In particular embodiments, administration of drug compositions is stopped to provide an anti-side effect. An anti-side effect can reduce or eliminate a negative effect of formulation administration such as engraftment-induced cytokine storm (cytokine release syndrome), tumor lysis syndromes (TLS) or B cell cytopenia.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of disease, stage of disease, previous or concurrent therapeutic interventions, idiopathy of the subject and route of administration.

Therapeutically effective amounts of modified formulations to administer can include greater than 10² cells, greater than 10³ cells, greater than 10⁴ cells, greater than 10⁵ cells, greater than 10⁶ cells, greater than 10⁷ cells, greater than 108 cells, greater than 109 cells, greater than 10¹⁰ cells, or greater than 10¹¹.

Useful doses to administer within modifying formulations or drug compositions can range from, for example, 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

(x) Kits. The current disclosure also includes kits assembled with materials useful to practice aspects of the disclosure. The kits can include, for example, cells (e.g., immune cells), nucleic acids encoding an activity-inducible fusion protein, transfection reagents, assay reagents, drug molecules, buffers, cell nutrients and expansion media, cell sorting molecules (e.g., Dynabeads), tubes, wells, and small molecule estrogen analogs (e.g., tamoxifen, 4-OHT, ES8, CMP8).

(xi) hsp90 Clients. Activity-inducible fusion proteins disclosed herein can include a binding domain derived from an alternative hsp90 client molecule or domain thereof. Hsp90 client proteins described herein are grouped according to transcription factors, kinases, and “other” as denoted in picard.ch/downloads/hsp90 interactors.

Examples of hsp90 client transcription factors include 12 (S)-HETE receptor; AF9/MLLT3; all vertebrate steroid receptors (GR, MR, ERa, ERB, PR, AR); AGL24; ATF3; BBX; BCL-6; Bclaf1; BES1; BrZ7; BZR1; C20orf194; CAR; CEBPE; Cwt1; CXXC1; cytoplasmic v-erbA; DLX6; DMRTA1; EcR; FOXD4L6; FOXM1; FOXP2; GTF2IRD2; Hap1; HCFC1; HMGA1, HMGA2; HNF4A; HP1BP3; HSF-1; HsfA1, HsfA2, HsfB1; IRF2; IRF3; ISX; LFY; MAFG; Mal63; MaIR; MAX; Met1; MeWRKY20; MKX; mod (mdg4); c-Myc; Nanog; NFIC; FRKB; Notch1 (ICN1); NR1H3; NR112; Oct4; p53; p73; PAS family members: Dioxin receptor (=AhR), Sim, HIF-1a, HIF-2a, HIF-3a; PCGF6; POGK; PPARa, PPARB, PPARy; PRDM1; PREB; PXR; REST; REV-ERBa; RlmA (of Aspergillus); SETDB1; SIM2; SLFN11; SOC1; SOX11; Sp1; SREBF1; SREBP1; SREBP2; Stat2; Stat3 (also in caveolin-1 complexes in rafts); Stat5; SUP; TADA2A; TBX22; TCF25; TDP-43; TEAD2; TFDP3; THAP4; TonEBP/OREBP; TRIM32; Tup1; Twist1; Ure2; USP1; VDR; VP16; water mold Achlya steroid (antheridiol) receptor; WT1; ZBED4; ZBTB17; ZBTB20; ZC3H7B; ZNF215; ZNF509; and ZNF74.

Examples of hsp90 client kinases include ACVR1B; ACVR1C; ACVR2B; Akt/PKB; AKT2; ALK; ALK1, ALK5; ALPK1; AMHR2; AMPKa, AMPKy; ARAF; ASK1; ATM; AURKC; Aurora B; AXL; Bcr-Abl; BCR-FGFR1; BGLF4 of EBV; BLK; BMPR1A; BMX; BTK; c-Abl; c-Kit; c-Mos; CAMK1G; CAMK2A; CAMK2B; CAMK2D; CAMK2G; CAMK4; CAMKK1; CAMKK2; CAMKV; casein kinase IIα catalytic subunit; Cdc2 (=Cdk1); CDK11B; CDK14; CDK15; CDK18; Cdk2, Cdk4, Cdk6, Cdk9, Cdk11; CDK3; CheA (E. coli); Chk1; Cla4; CLK2; CLK3; Cot=Tpl-2; CSF1R; CSNK1A1; DCLK2; DDR1; DDR2; Death-associated kinases DAPK, DAPK2, DAPK3; DLK; DMPK; DYRK1B; DYRK2; DYRK4; eEF-2 kinase; EGF receptor (mutant and wt); elF2-a kinases HRI, Gcn2, Perk, PKR; Eml4-Alk; EPHA1; EphA2; EPHA4; EPHB1; EPHB6; ErbB2; ERBB3; ERBB4; ERK5; FASTK; FGFR1; FGFR3 and FGFR4; Flt3; FLT4; FOP2-FGFR1; FRK; Fused; FYN; Gal1; GRK2 and GRK6; GRK4; GRK7; GSK3A; GSK3B; HCK; HER3; HIPK4; HopBF1 effectors; ICK; INSRR; Insulin receptor; Insulin-like growth factor 1 receptor; Integrin-linked kinase; IP6K2; IRAK-1; IRAK2; IRAK3; Ire1a; ITK; IKB kinases (IKK) a, B, y, ¿; JAK1; JNK; KSR; LATS1, LATS2; LCK; LIMK1; LIMK2; Lkb1; LMTK3; LRRK2; LYN; MAP2K5; MAP2K7; MAP3K12; MAP3K15; MAP3K2; MAP3K6; MAP3K9; MAP4K1; MAP4K2; MAP4K4; MAPKKK (MEKK) YODA; MAPK15; MAPK4; MAPK6; MAPK7; MAST1; MAST2; MATK; MEK; MEKK1 and MEKK3; MERTK; MET; Mik1; MINK1; MLK3; MLKL; MOK, MAK, MRK; MpkA (of Aspergillus); Mps1; mTOR; MUSK; MYLK2; MYLK3; MYLK4; NEK11; NEK8; NEK9; NIK; NPM-AIk; NPR2; NTRK1; NTRK2; NTRK3; NUAK2; Nucleophosmin-Anaplastic Lymphoma Kinase; p38; p90RSK; PAK6; PASK; Pbs2; PDGFRB; PDIK1L; PDK1; PGK1; PI4KIIB; Pim-1; PIM2; PIM3; Pink1; PKCA, PKCsand other PKCs; PKM2; PKN1; PKN2; platelet-derived growth factor receptor a; Plk1; Plk3; Pnck; pp60v-src, c-src; PRKAA2; PRKACB; PRKCA; PRKCB; PRKCG; PRKCH; PRKCI; PRKCQ; PRKCZ; PRKD1; PRKD2; PRKDC; PRKG2; PRKX; PRKY; PSKH1; PSKH2; PTK2; PTK2B; PTK6; PTK6; Raf-1, B-Raf, Ste11; RET; RET/PTC1; RIP1; RIP3; Ron; ROR2; RPS6KA1; RPS6KA2; RPS6KA3; RPS6KA5; RPS6KA6; RPS6KB1; RPS6KC1; RPS6KL1; Ryk; SGK-1; SGK2; SGK223; SGK3; SIt2; src related tyrosine kinases: fer, fes, fgr, fps, Ick, yes; SRPK1; SRPK3; SSCMK1; STK32B; STK32C; STK33; STK38; STK38L; STYK1; SYK; TAK1; TAOK3; TBK1; TESK1; TESK2; TGFβ receptors I and II; TIE1; TNK1; TNK2; TNNI3K; TP53RK; TrkAl and III; TrkB; TSSK1B; TSSK2; TSSK3; TSSK4; TSSK6; Tyk2; TYRO3; Ulk1; VEGFR1, VEGFR2; Wee1, Swe1; WNK4; and ZAP-70.

Examples of hsp90 client molecules denoted as “other” include Act1 (=TRAF3IP2); Adenosine A2A receptor; a2C adrenergic receptor; AID; AIP56; Aldo-keto reductase 1B10; ANAPC2; ANKMY2; Annexin A2; ANP receptor; ANP32C/D; Apaf-1; apoB; APOBEC-3B,-3C,-3G; Arb1; ARD1; Argonaute-1 (Ago1); Argonaute-2 (=Ago2=GERp95); Argonaute-4 (Ago4); ARMC5; ArtAB; ASB17; ASB2; ASB3; ASB4; ASB6; ATG8 (GABARAP) proteins; Axin 1; BCAP (PIK3AP1); BALF5 of EBV; Bcl-2; Bcl-xL; Beclin 1; Bid; BIN2; BLM helicase; Bms1; BPIFB4; BRAT1; BRCA1; BRCA2; BRMS1; BTRC; c-IAP1; calcineurin (Cna2; catalytic subunit); calmodulin; calmodulin methyltransferase; calpain-1; calponin; CARM1; Caspase-8; β-catenin; CB2 cannabinoid receptor; Ccp1; CCDCl17; CD38 type III; CD79a; Cdc13; Cdc14; Cdc25a and Cdc25c; Cdk5 activator p35; CPEB1, CPEB2, CPEB3; CFTR (nascent and mutant polypeptide); ChAT; CheZ (E. coli); Chl1; Chronophin; Cineole synthase 1; Clathrin heavy chain; CLC-1 chloride channel; CLC-2 chloride channel; Clostridium toxin CDT; Clostridium toxin iota; Clusterin; COG complex; COI1; Complement C9; Cry toxins; CTA1=CtxA1; Ctf13/Skp1 component of CBF3; CUL1; CUL2; CUL3; CUL4A; CUL4B; Cup; cyclin B; cyclophilin D (mitochondrial); Cyr1; cytoskelettal proteins: actin, tubulin (including ciliary (4-tubulin), myosin (including Myo3B); DBC2; DEDD; Dengue virus protein E; Dengue virus proteins NS1/2B/3/4B/5; DET1; Diphtheria toxin A; DNA helicase Ssl2; DNA polymerase a; DNA polymerase λ; DNA polymerase n; DnaA (E. coli); DNMT1; Dsn1; DTX4; E6\hat{ }E7; EBAX-1; Emc2; ENC1; eNOS, nNOS (?); ether-a-gogo-related potassium channel (ERG=HERG=KCNH2); EZH2; F1F0-ATP synthase; FANCA; FBXL12; FBXL13; FBXL14; FBXL15; FBXL18; FBXL2; FBXL3; FBXL6; FBXL8; FBXO10; FBXO17; FBXO18; FBXO24; FBXO25; FBXO27; FBXO28; FBXO3; FBXO34; FBXO38; FBXO4; FBXO40; FBXO6; FBXO9; FBXW11; FBXW2; FBXW5; FBXW7; FGAMS; Fibronectin; FliN, Flil (E. coli); FLIPs and FLIPL; Folliculin; free By subunit of G protein; FtsZ; G2E3; GAN; Gln1; GLT-1; GluR1; glutathione S-transferase subunit 3 (KS type); Guanylate cyclase, soluble; Gao, Ga12; Glucocerebrosidase; GREB1; HAX-1; HDAC1; HDAC6; HECTD3; Hepatitis B virus core protein; Hepatitis C virus protein NS3; Hepatitis E virus capsid protein; HERC4; HERC6; Histones H1, H2A, H2B, H3 and H4; HMGCR; Hsp27; Humanin; Huntingtin; Importin 4 (IPO4); Importin a1; Importin-α6 (KPNA5); Ino80; Inositol 1,4,5-trisphosphate receptor 3; Integrin a2; Integrin a4; Integrin aL; IL-1B; IRS-2; Japanese encephalitis virus E protein; JIpA; KAP1; KAT5; KBTBD4; KBTBD7; KCNA5; KCNA6; KCNG1; KCNS3; KCNQ4; KCTD8; KDM3A/JMJD1A; KDM4B/JMJD2B; KEAP1; KIAA0317; Kir6.2; KLHL1; KLHL10; KLHL13; KLHL14; KLHL15; KLHL22; KLHL23; KLHL25; KLHL26; KLHL29; KLHL32; KLHL34; KLHL36; KLHL38; KLHL6; knob complexes (in the membrane of Plasmodium-infected erythrocytes); KSHV K1; KSR1; KSR2; L protein of HRSV; Lamin A/C; LAMP-2A; LANA of KS-HSV; LAP; LARP4B; Legumain; LGALS3BP; LIS1; LNX1; LOC440248; LOX1 (OLR1); LOXL2; Lpl1 (S. aureus); LRP1 (=CD91); LRP5; LRSAM1; LSD1; LSM8; macromolecular aminoacyl-tRNA synthetase complex; Macrophage scavenger receptor; MAP1B; MARCH9; Mdm2; MDM4; MeCatalase1; Mg2+-dependent phosphatidate phosphohydrolase; MIF; misfolded VHL; MMP2, MMP3, MMP9; μ-opioid receptor; MRE11/Rad50/NBS1 (MRN) complex; MRP1; Msps/XMAP215/ch-TOG; MTA1; MTG8; MUC1; Myoglobin; N-myc downstream-regulated gene 1 (NRDG1); N-WASP; Na⁺-K⁺—Cl⁻ cotransporter 1; NadA; NAP1; NB-LRR proteins: RPM1 and RPS2, Nod1, Nod2, NALP2, NALP3, NALP4, NALP12, IPAF, RPP4; NCC; NDRG2; NELF-E; Nervous necrosis virus capsid protein; Neuraminidase; Neuropeptide Y; NHE1; NHLRC1; Nibrin; NleH1 and NleH2; NMNAT2; Norovirus capsid protein VP1; Nox1, Nox2, Nox3, Nox5; NS1; Nsl1; nsP3 and nsP4 of Chikungunya virus; Nucleoprotein (NP) of MERS-COV; Nup62; OGT; OsCERK1; P protein (rabies virus); P1 (picornaviral capsid precursor protein P1); p14ARF; P2X7 purinergic receptor; p300; P450 CYP2E1; PARK2; PARK7 (DJ-1); PB1 and PB2 subunits of influenza RNA pol.; PCGF1; PCGF3; PCNA; Peli1; perilipin; PfCRT; PIDD; Piwi; PIWIL2; PLCy; PLN; polysomal ribonuclease 1 (PMR1); PPAT; PRDM14; PRMT5; pro-Dcp1; prolactin receptor; prostacyclin synthase; proteasome; PRPF8; PRPF19; PTPN22; Ptx; R-protein I-2; R2TP complex through Pih1; Rab-aGDI; Rab3a; Rab11a; RAB40A; Rac/Rop GTPase Rac1 (rice); Rac1; Rad51; Rad52; RAG1; Ral-binding protein 1 (RalBP1); RanBP9; Rapsyn; Raptor; RCBTB1; RCBTB2; reovirus protein σ1; REV1; reverse transcriptase of hepatitis B virus; RFWD3; RGS11; RGS6; RGS7; RGS9; RHOBTB1; ribosomal protein L2 (E. coli); ribosomal proteins S3 and S6; ricin catalytic A chain; RIG-I; RNA-dep. RNA polymerase (of Bamboo mosaic virus); RNF10; RNF111; RNF19B; RNF40; RNGTT; Rnr4; Rpb1; SCAP; SDF2; SENP3; SERCA2a; SERT (SLC6A4); SF3B3; SH3RF2; Sicily; SIR2 (SIR2RP1 in Leishmania); SIRT1; SIRT2; SKP2; SKP2 complexes; SLC6A14; SMYD1, SMYD2, SMYD3; snoRNP complexes; SNRNP200; SOCS6; SPSB1; SPSB3; SREC-I; STING; SUR1 (subunit of β-cell ATP-sensitive potassium channel); survivin; SV40 large T-antigen; Swr1; α-synuclein; Tab2/3; Tas3; Tau protein; Tax; TCL1A; telomerase; TFR1; thiopurine S-methyltransferase; thrombin receptor (PAR-1); thromboxane synthase; TilS; TIMP2; TIR1; Tissue plasminogen activator (tPA); Titin; TLR4/MD-2 complex; TLR7; TLR9; Tm-22; TNFAIP3; TOM40; TRIM10; TRIM17; TRIM2; TRIM36; TRIM37; TRIM41; TRIM49; TRIM56; TRIM7; TRIM73; TRIM74; TRIM8; Triosephosphate isomerase; Trithorax (and ortholog MLL); Trx1; TrxR; TSG101; Tyrosine hydroxylase; UCH-L1; UHRF1; Ulp1; uPA; Ura2; URI complex; Uroporphyrinogen decarboxylase (HemE) [in cyanobacteria]; Us11 (of HSV-1); USP19; Utp21; Vaccinia core protein 4a; vFLIP (of KSHV); Vimentin; VIP1; VPS18; VPS41; WASF3; WSB2; WTAP; WWP1; XPO1; XPORT; XRCC1; ZEITLUPE; and ZMYND10.

(xii) Variants. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (IIe), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, IIe, Phe, Met, and Trp; Group 11 (aliphatic): Gly, Ala, Val, Leu, and IIe; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: IIe (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); IIe (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within +2 is preferred, those within +1 are particularly preferred, and those within +0.5 are even more particularly preferred.

As outlined above, amino acid substitutions may be based on the relative similarity of the amino acid side-chain substituents, for example, their hydrophobicity, hydrophilicity, charge, size, and the like.

Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in a cell activation study. Functional variants and functional fragments of intracellular domains (e.g., intracellular signaling domains) transmit activation or inhibition signals comparable to a wild-type reference when in the activated state of the current disclosure. Functional variants and functional fragments of binding domains bind their cognate antigen or ligand at a level comparable to a wild-type reference.

In particular embodiments, a binding domain VH region can be derived from or based on a VH of a known antibody and can optionally contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the known antibody. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a VL region in a binding domain is derived from or based on a VL of a known antibody and optionally contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known antibody. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

These considerations similarly apply to TCR chains and other derived binding domains.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

### (XIII) EXEMPLARY EMBODIMENTS

1. An activity-inducible fusion protein including a transcription factor and an hsp90 binding domain.

2. The activity-inducible fusion protein of embodiment 1, wherein the transcription factor initiates gene transcription resulting in immune cell activation.

3. The activity-inducible fusion protein of embodiment 2, wherein the transcription factor is STAT5a and the STAT5a initiates gene transcription of Bcl2, Junb, NDRG1, Id2, DNAJC6, CBS, PPP2R2B, ST3GAL1, SAMD4A, SSH2, and MAP3K5.

4. The activity-inducible fusion protein of embodiment 2, wherein the transcription factor is STAT5b and STAT5b initiates gene transcription of DOCK8, SNX9, LNPEP, SKAP1, PTGER1, and FOXP3.

5. The activity-inducible fusion protein of embodiment 2, wherein the transcription factor is STAT3 and STAT3 initiates gene transcription of c-Fos, HIF-1a, c-Myc, Sox2, Zeb1, Bcl-2, Mcl-1, and Bcl-xL.

6. The activity-inducible fusion protein of embodiment 2, wherein the immune cell activation is evidenced by increased proliferation, cytokine release, and/or target cell killing.

7. The activity-inducible fusion protein of embodiment 1 or 2, wherein the transcription factor is selected from ca-STAT3, caSTAT5a, caSTAT5b, JUN (c-Jun), c-Myc, TCF7, BCL-6, STAT6, STAT5a, STAT5b, STAT3, c-Fos, HIF-1a, Sox2, Zeb1, Bcl-2, Mcl-1, Bcl-xL, Junb, FOXP3, Max, E2F (E2F1, EN2F2, and E2F3a), AP-1, NF-kb, FOX including FOXF2 and FoxO, Sp1, Sp2, Sp3, Sp4, Sp5, Sp6 (KLF14), Sp7, Sp8, Sp9, KLF1, KLF2, KLF3, KLF4, KLF5, KLF6, KLF7, KLF8, KLF9, KLF10, KLF11, KLF12, KLF13, KLF15, KLF16, and KLF17 8. The activity-inducible fusion protein of embodiment 7, wherein the transcription factor is selected from ca-STAT3, caSTAT5a, caSTAT5b, STAT6, AP-1, c-Myc, TCF7, or BCL-6.

9. The activity-inducible fusion protein of embodiment 7, wherein the transcription factor is selected from human ca-STAT3, human caSTAT5a, human caSTAT5b, human STAT6, human AP-1, human c-Myc, human TCF7, or human BCL-6.

10. The activity-inducible fusion protein of embodiment 8, wherein ca-STAT3 includes the sequence as set forth in SEQ ID NO: 136, caSTAT5a includes the sequence as set forth in SEQ ID NO: 138, and/or caSTAT5b includes the sequence as set forth in SEQ ID NO: 141 or wherein ca-STAT3 includes a sequence having at least 90% sequence identity to the sequence as set forth in SEQ ID NO: 136, caSTAT5a includes a sequence having at least 90% sequence identity to the sequence as set forth in SEQ ID NO: 138, and/or caSTAT5b includes a sequence having at least 90% sequence identity to the sequence as set forth in SEQ ID NO: 141.

11. The activity-inducible fusion protein of any of embodiments 1-10, wherein the hsp90 binding domain binds hsp90 with a lower affinity than it binds a drug molecule.

12. The activity-inducible fusion protein of any of embodiments 1-11, wherein the hsp90 binding domain includes a hormone binding domain.

13. The activity-inducible fusion protein of embodiment 12, wherein the hormone binding domain is an engineered estrogen receptor binding domain (EBD).

14. The activity-inducible fusion protein of embodiment 13, wherein the EBD includes the binding domain portion of the estrogen receptor and a set of mutations selected from G521R; E353A; L384M and M421G; L384M, M421G, and G521R; or G400V, M543A, and L544A.

15. The activity-inducible fusion protein of embodiment 14, wherein the EBD has the sequence as set forth in SEQ ID NO: 4, SEQ ID NO: 6, SEQ ID NO: 9, SEQ ID NO: 11, or SEQ ID NO: 13 or has a sequence having at least 90% sequence identity to the sequence as set forth in SEQ ID NO: 4, SEQ ID NO: 6, SEQ ID NO: 9, SEQ ID NO: 11, or SEQ ID NO: 13.

16. The activity-inducible fusion protein of embodiment 13, wherein the EBD is ERT2 and the transcription factor is selected from ca-STAT3, caSTAT5a, caSTAT5b, STAT6, AP-1, c-Myc, TCF7, or BCL-6.

17. The activity-inducible fusion protein of embodiment 16, wherein the EBD is ERT2 and the transcription factor is selected from human ca-STAT3, human caSTAT5a, human caSTAT5b, human STAT6, human AP-1, human c-Myc, human TCF7, or human BCL-6.

18. The activity-inducible fusion protein of any of embodiments 11-17, wherein the drug molecule includes a small molecule estrogen analog.

19. The activity-inducible fusion protein of embodiment 18, wherein the small molecule estrogen analog includes tamoxifen, a salt of tamoxifen, a metabolite of tamoxifen, or a compound that is structurally similar to tamoxifen.

20. The activity-inducible fusion protein of embodiments 18 or 19, wherein the small molecule estrogen analog includes tamoxifen, 4-OHT, ES8, or CMP8.

21. The activity-inducible fusion protein of any of embodiments 1-20, wherein the fusion protein includes a linker that links the transcription factor to the hsp90 binding domain.

22. The activity-inducible fusion protein of embodiment 20, wherein the linker includes (Gly₄Ser)ₙ (SEQ ID NO: 148), (Gly₃Ser), (SEQ ID NO: 150), (GGGG), (SEQ ID NO: 151), (GGG)ₙ, or (GSAGSAAGSGEF), (SEQ ID NO: 152) wherein n is an integer of 1, 2, 3, 4, 5, 6, 7, 8, 9, or 10.

23. The activity-inducible fusion protein of any of embodiments 1-22, wherein the activity-inducible fusion protein is co-expressed with a chimeric antigen receptor (CAR), wherein when expressed by a cell, the CAR includes:
an extracellular component and an intracellular component linked by a transmembrane domain, wherein
   the extracellular component includes a ligand binding domain and
   the intracellular component includes an intracellular signaling domain.

24. The activity-inducible fusion protein of embodiment 23, wherein the ligand binding domain binds a cancer antigen or a viral antigen.

25. The activity-inducible fusion protein of embodiments 23 or 24, wherein the ligand binding domain includes an scFv that binds HER2, CE7, hB7H3, EGFR, EGFRvIII, CD19, CD20, CD22, EphA2, IL13Ra2, L1CAM, oaGD2, B7H3, CD33, Mesothelin, ROR1, FITC or VAR2CSA.

26. The activity-inducible fusion protein of embodiment 25, wherein the scFv has a sequence as set forth in SEQ ID NO: 16, SEQ ID NO: 18, SEQ ID NO: 19, SEQ ID NO: 20, SEQ ID NO: 21, SEQ ID NO: 23, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 342, or SEQ ID NO: 43 or a sequence having at least 90% sequence identity to the sequence as set forth in SEQ ID NO: 16, SEQ ID NO: 18, SEQ ID NO: 19, SEQ ID NO: 20, SEQ ID NO: 21, SEQ ID NO: 23, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 342, or SEQ ID NO: 43.

27. The activity-inducible fusion protein of embodiment 23, wherein the ligand binding domain binds an immune cell antigen.

28. The activity-inducible fusion protein of embodiment 27, wherein the immune cell antigen is expressed by a B cell, a T cell, a natural killer cell, a natural killer T cell, a MAIT cell, a myeloid cell, a macrophage, a monocyte, or a dendritic cell.

29. The activity-inducible fusion protein of embodiment 23, wherein the ligand binding domain binds a hapten.

30. The activity-inducible fusion protein of embodiment 29, wherein the hapten includes fluorescein, urushiol, quinone, biotin, or dinitrophenol.

31. The activity-inducible fusion protein of embodiments 29 or 30, wherein the ligand binding domain is an scFv having the sequence as set forth in SEQ ID NO: 44, SEQ ID NO: 45, SEQ ID NO: 46, SEQ ID NO: 47, SEQ ID NO: 48, SEQ ID NO: 49, SEQ ID NO: 51, SEQ ID NO: 53, SEQ ID NO: 60, SEQ ID NO: 61, SEQ ID NO: 62, SEQ ID NO: 63, SEQ ID NO: 64, or SEQ ID NO: 65 or a sequence having at least 90% sequence identity to the sequence as set forth in SEQ ID NO: 44, SEQ ID NO: 45, SEQ ID NO: 46, SEQ ID NO: 47, SEQ ID NO: 48, SEQ ID NO: 49, SEQ ID NO: 51, SEQ ID NO: 53, SEQ ID NO: 60, SEQ ID NO: 61, SEQ ID NO: 62, SEQ ID NO: 63, SEQ ID NO: 64, or SEQ ID NO: 65.

32. The activity-inducible fusion protein of any of embodiments 23-31, wherein the extracellular component further includes a spacer region.

33. The activity-inducible fusion protein of embodiment 32, wherein the spacer region includes an IgG4 hinge.

34. The activity-inducible fusion protein of any of embodiments 23-33, wherein the intracellular signaling domain includes a CD3Z signaling domain.

35. The activity-inducible fusion protein of any of embodiments 23-34, wherein the intracellular signaling domain includes a 4-1BB signaling domain.

36. The activity-inducible fusion protein of any of embodiments 23-35, wherein the intracellular signaling domain includes a CD37 signaling domain and a 4-1BB signaling domain.

37. The activity-inducible fusion protein of any of embodiments 23-36, wherein the transmembrane domain includes a CD28 transmembrane domain.

38. The activity-inducible fusion protein of embodiment 23, wherein the intracellular signaling domain further includes a co-stimulatory immune molecule selected from 4-1BB, OX40, CD40, CD30, CD27, DR3, SLAMF1, ICOS, GITR, CD25, CD28, CD79A, CD79B, CD226, CARD11, DAP10, DAP12, DR3, FcRa, FcRB, FcRy, Fyn, Lck, LAT, LRP, LIGHT, NKG2D, NOTCH1, NOTCH2, NOTCH3, NOTCH4, ROR2, Ryk, Slp76, pTa, TCRa, TCRB, TIM1, TRIM, Zap70, an PTCH2.

39. A nucleotide encoding an activity-inducible fusion protein of any of embodiments 1-38.

40. A cell genetically modified to express an activity-inducible fusion protein of any of embodiments 1-38.

41. The cell of embodiment 40, genetically modified to express at least two types of an activity-inducible fusion protein of any of embodiments 1-38, wherein the two types have different transcription factors and different hsp90 binding domains that bind different drug molecules.

42. The cell of embodiment 40 or 41, wherein the cell is a T cell or a natural killer (NK) cell.

43. The cell of embodiment 42, wherein the T cell is a CD4+ or a CD8+ T cell.

44 The cell of embodiment 40 or 41, wherein the cell is an induced pluripotent stem cell (iPSC), a tumor-infiltrating lymphocyte (TIL), a marrow-infiltrating lymphocyte (MIL), a natural killer T cell (NKT), a mucosal-associated invariant T (MAIT) cell, a dendritic cell, a monocyte or a macrophage.

45. A system for altering the activation state of an immune cell including:
the cell of any of embodiments 40-44; and
the drug molecule.

46. The system of embodiment 45, wherein the cell is ex vivo or in vivo.

47. The system of embodiments 45 or 46, formulated for administration to a subject.

48. The system of any of embodiments 45-47, wherein the formulated system includes
(i) ex vivo manufactured cells expressing the activity-inducible fusion protein formulated into a pharmaceutically acceptable carrier to create a modified formulation; and/or
(ii) cell-targeted viral vectors and/or a cell-targeted nanoparticles formulated into a pharmaceutically acceptable carrier to create a modifying formulation wherein the cell-targeted viral vectors and/or a cell-targeted nanoparticles include gene-modifying components that result in expression of the activity-inducible fusion protein in vivo by the targeted cell following administration; and
(iii) the drug molecule formulated into a pharmaceutically acceptable carrier to create a drug composition.

49. A method of treating a subject in need thereof including administering a system of any of embodiments 45-48 to the subject, thereby treating the subject.

50. The method of embodiment 49, wherein the administering includes administering the modified formulation and the drug composition.

51. The method of embodiment 49, wherein the administering includes administering the modifying formulation and the drug composition.

52. The method of any of embodiments 49-51, wherein the method further includes stopping administration of the drug molecule to reduce a side effect of system administration.

53. The method of any of embodiments 49-52, wherein the method further includes stopping administration of the drug molecule when the subject is no longer in need thereof.

54. The method of any of embodiments 49-53, wherein the system includes at least two types of an activity-inducible fusion protein, wherein the two types have different hsp90 binding domains that bind different drug molecules.

55. The method of embodiment 54, including administering at least two types of drug molecules wherein one of the at least two types binds the hsp90 binding domain of one activity-inducible fusion protein of the system and wherein one of the at least two types binds the hsp90 binding domain of a different activity-inducible fusion protein of the system.

56. The method of embodiment 55, wherein the method further includes stopping administration of at least one of the drug molecules.

57. The method of embodiments 55 or 56, wherein the method further includes stopping administration of all of the drug molecules.

58. The method of any of embodiments 54-55, wherein the two types further have different transcription factors.

59. The method of any of embodiments 49-58, wherein the subject is in need thereof due to cancer or an infection.

60. The method of embodiment 59, wherein the cancer is bladder cancer, head and neck cancer, breast cancer, colon cancer, kidney cancer, liver cancer, lung cancer, ovarian cancer, prostate cancer, pancreatic cancer, stomach cancer, cervical cancer, thyroid cancer skin cancer, hematopoietic cancer of a lymphoid lineage, hematopoietic cancer of myeloid lineage, neuroblastoma, glioma; tumors of the central and peripheral nervous system, including astrocytoma, neuroblastoma, or glioma.

61. The method of embodiment 59, wherein the cancer is acute lymphoblastic leukemia (ALL), acute myeloid leukemia (AML), or a brain cancer.

62. The method of embodiment 59, wherein the infection is a bacterial, viral, fungal, parasitic, or arthropod infection.

63. The activity-inducible fusion protein of any of embodiments 1-38, wherein the activity inducible fusion protein does not include a degron sequence.

(xiv) Experimental Examples. caSTAT5aER Variants Testing by Luciferase Assay. The STAT5 reporter cells 293t/STAT5-RE-Luc2P were seeded in a 96-well plate. Plasmid DNA of STAT5aER (T2) variants were transfected into these cells in combination with pRL-SV40 at 10:1 molar ratios using Lipofectamine 2000. The pRL-SV40 was a renilla luciferase plamid control that was used to normalize the transfection variation. Then the cells were treated with four doses of 4-hydroxy tamoxifen (4-OHT). 24-h later, Dual-Glo luciferase assay was performed according to the protocol from Promega. *ER (T2) contains three mutations, G400V/L539A/L540A, which makes it sensitive to 4-OHT and insensitive to 17β-oestradiol.

Phospho-Western Blot. H9 T cells were transduced with CA-STAT5a-ER (T2) or CA-STAT5a and seeded in a 6-well plate. 24 h after 4-OHT or vehicle treatment, cells were harvested, and lysed in 100 ul of RIPA buffer with protease inhibitor cocktail. The cell lysate was mixed with 4× Laemmili buffer (+β-mercaptoethanol), boiled at 95° C. for 5 min and loaded to a 4-12% NuPAGE gel. The western blotting standard operating procedure was followed using rabbit anti human P-STAT5 694Y and mouse anti-β-Actin Ab Abs.

Growth Curve. Human primary CD8 T cells were co-transduced with a CD19CAR-EGFRt and Her2tG or caSTAT5aER-Her2tG construct. The transduced cells were sorted using anti-EGFRt-Biotin and anti-Biotin microbeads and then cultured in RPMI/10% FBS media supplemented with or without interleukin (IL) 2 and IL15 for growth curve analysis. The caSTAT5aER transduced cells were treated with 100 nM of 4-OHT every 3-4 days and on day 19 changed to 500 nM 4-OHT.

(xv) Closing Paragraphs. The nucleic acid and amino acid sequences provided herein are shown using letter abbreviations for nucleotide bases and amino acid residues, as defined in 37 C.F.R. § 1.822 and set forth in the tables in WIPO Standard ST.25 (1998), Appendix 2, Tables 1 and 3. Only one strand of each nucleic acid sequence is shown, but the complementary strand is understood as included in embodiments where it would be appropriate.

The terms “specific binding affinity” or “specifically binds” or “specific binding” or “specifically targets” as used herein, describe binding of one molecule to another at greater binding affinity than background binding. A binding domain (e.g., of a CAR including a binding domain) “specifically binds” to a target molecule if it binds to or associates with a target molecule with an affinity or Ka (i.e., an equilibrium association constant of a particular binding interaction with units of 1/M) of, for example, greater than or equal to 10⁵ M⁻¹. In particular embodiments, a binding domain (or CAR) binds to a target with a Ka greater than or equal to 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (Kd) of a particular binding interaction with units of M (e.g., 10-5 M to 10-13 M, or less). Affinities of binding domains and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, N.J., or optical biosensor technology such as the EPIC system or EnSpire that are available from Corning and Perkin Elmer respectively (see also, e.g., Scatchard et al. (1949) Ann. N.Y. Acad. Sci. 51:660; U.S. Pat. Nos. 5,283,173; 5,468,614).

In particular embodiments, the affinity of specific binding is 2 times greater than background binding, 5 times greater than background binding, 10 times greater than background binding, 20 times greater than background binding, 50 times greater than background binding, 100 times greater than background binding, or 1000 times greater than background binding or more.

“Derived from” as used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3ζ molecule, the intracellular signaling domain retains sufficient CD3ζ structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3Z sequence and delete unwanted sequence, or impose mutations, to arrive at the intracellular signaling domain.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to activate a cell expressing an activity-inducible fusion protein in the presence of its relevant drug molecule and relevant physiological condition (e.g., antigen binding for a CAR; ligand binding for a co-stimulatory or inhibitory molecule).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. An activity-inducible fusion protein comprising a transcription factor and an hsp90 binding domain.

2. The activity-inducible fusion protein of claim 1, wherein the transcription factor initiates gene transcription.

3. The activity-inducible fusion protein of claim 2, wherein the transcription factor is STAT5a and the STAT5a initiates gene transcription of Bcl2, Junb, NDRG1, Id2, DNAJC6, CBS, PPP2R2B, ST3GAL1, SAMD4A, SSH2, and MAP3K5.

4. The activity-inducible fusion protein of claim 2, wherein the transcription factor is STAT5b and STAT5b initiates gene transcription of DOCK8, SNX9, LNPEP, SKAP1, PTGER1, and FOXP3.

5. The activity-inducible fusion protein of claim 2, wherein the transcription factor is STAT3 and STAT3 initiates gene transcription of c-Fos, HIF-1a, c-Myc, Sox2, Zeb1, Bcl-2, Mcl-1, and Bcl-xL.

6. The activity-inducible fusion protein of claim 2, wherein the immune cell activation is evidenced by increased proliferation, cytokine release, and/or target cell killing.

7. The activity-inducible fusion protein of claim 1, wherein the transcription factor is selected from ca-STAT3, caSTAT5a, caSTAT5b, JUN (c-Jun), c-Myc, TCF7, BCL-6, STAT6, STAT5a, STAT5b, STAT3, c-Fos, HIF-1a, Sox2, Zeb1, Bcl-2, Mcl-1, Bcl-xL, Junb, FOXP3, Max, E2F (E2F1, EN2F2, and E2F3a), AP-1, NF-kb, FOX comprising FOXF2 and FoxO, Sp1, Sp2, Sp3, Sp4, Sp5, Sp6 (KLF14), Sp7, Sp8, Sp9, KLF1, KLF2, KLF3, KLF4, KLF5, KLF6, KLF7, KLF8, KLF9, KLF10, KLF11, KLF12, KLF13, KLF15, KLF16, and KLF17

8. The activity-inducible fusion protein of claim 7, wherein the transcription factor is selected from ca-STAT3, caSTAT5a, caSTAT5b, STAT6, AP-1, c-Myc, TCF7, or BCL-6.

9. The activity-inducible fusion protein of claim 7, wherein the transcription factor is selected from human ca-STAT3, human caSTAT5a, human caSTAT5b, human STAT6, human AP-1, human c-Myc, human TCF7, or human BCL-6.

10. The activity-inducible fusion protein of claim 8, wherein ca-STAT3 comprises the sequence as set forth in SEQ ID NO: 136, caSTAT5a comprises the sequence as set forth in SEQ ID NO: 138, and/or caSTAT5b comprises the sequence as set forth in SEQ ID NO: 141.

11. The activity-inducible fusion protein of claim 1, wherein the hsp90 binding domain binds hsp90 with a lower affinity than it binds a drug molecule.

12. The activity-inducible fusion protein of claim 1, wherein the hsp90 binding domain comprises a hormone binding domain.

13. The activity-inducible fusion protein of claim 12, wherein the hormone binding domain is an engineered estrogen receptor binding domain (EBD).

14. The activity-inducible fusion protein of claim 13, wherein the EBD comprises the binding domain portion of the estrogen receptor and a set of mutations selected from G521R; E353A; L384M and M421G; L384M, M421G, and G521R; or G400V, M543A, and L544A.

15. The activity-inducible fusion protein of claim 14, wherein the EBD has the sequence as set forth in SEQ ID NO: 4, SEQ ID NO: 6, SEQ ID NO: 9, SEQ ID NO: 11, or SEQ ID NO: 13.

16. The activity-inducible fusion protein of claim 13, wherein the EBD is ERT2 and the transcription factor is selected from ca-STAT3, caSTAT5a, caSTAT5b, STAT6, AP-1, c-Myc, TCF7, or BCL-6.

17. The activity-inducible fusion protein of claim 16, wherein the EBD is ERT2 and the transcription factor is selected from human ca-STAT3, human caSTAT5a, human caSTAT5b, human STAT6, human AP-1, human c-Myc, human TCF7, or human BCL-6.

18. The activity-inducible fusion protein of claim 11, wherein the drug molecule comprises a small molecule estrogen analog.

19. The activity-inducible fusion protein of claim 18, wherein the small molecule estrogen analog comprises tamoxifen, a salt of tamoxifen, a metabolite of tamoxifen, or a compound that is structurally similar to tamoxifen.

20. The activity-inducible fusion protein of claim 18, wherein the small molecule estrogen analog comprises tamoxifen, 4-OHT, ES8, or CMP8.

21. The activity-inducible fusion protein of claim 1, wherein the fusion protein comprises a linker that links the transcription factor to the hsp90 binding domain.

22. The activity-inducible fusion protein of claim 20, wherein the linker comprises (Gly₄Ser)ₙ (SEQ ID NO: 148), (Gly₃Ser), (SEQ ID NO: 150), (GGGG), (SEQ ID NO: 151), (GGG)ₙ, or (GSAGSAAGSGEF), (SEQ ID NO: 152) wherein n is an integer of 1, 2, 3, 4, 5, 6, 7, 8, 9, or 10.

23. The activity-inducible fusion protein of claim 1, wherein the activity-inducible fusion protein is co-expressed with a chimeric antigen receptor (CAR), wherein when expressed by a cell, the CAR comprises:
an extracellular component and an intracellular component linked by a transmembrane domain, wherein
the extracellular component comprises a ligand binding domain and
the intracellular component comprises an intracellular signaling domain.

24. The activity-inducible fusion protein of claim 23, wherein the ligand binding domain binds a cancer antigen or a viral antigen.

25. The activity-inducible fusion protein of claim 23, wherein the ligand binding domain comprises an scFv that binds HER2, CE7, hB7H3, EGFR, EGFRvIII, CD19, CD20, CD22, EphA2, IL13Ra2, L1CAM, oaGD2, B7H3, CD33, Mesothelin, ROR1, FITC, or VAR2CSA.

26. The activity-inducible fusion protein of claim 25, wherein the scFv has a sequence as set forth in SEQ ID NO: 16, SEQ ID NO: 18, SEQ ID NO: 19, SEQ ID NO: 20, SEQ ID NO: 21, SEQ ID NO: 23, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 342, or SEQ ID NO: 43.

27. The activity-inducible fusion protein of claim 23, wherein the ligand binding domain binds an immune cell antigen.

28. The activity-inducible fusion protein of claim 27, wherein the immune cell antigen is expressed by a B cell, a T cell, a natural killer cell, a natural killer T cell, a MAIT cell, a myeloid cell, a macrophage, a monocyte, or a dendritic cell.

29. The activity-inducible fusion protein of claim 23, wherein the ligand binding domain binds a hapten.

30. The activity-inducible fusion protein of claim 29, wherein the hapten comprises fluorescein, urushiol, quinone, biotin, or dinitrophenol.

31. The activity-inducible fusion protein of claim 29, wherein the ligand binding domain is an scFv having the sequence as set forth in SEQ ID NO: 44, SEQ ID NO: 45, SEQ ID NO: 46, SEQ ID NO: 47, SEQ ID NO: 48, SEQ ID NO: 49, SEQ ID NO: 51, SEQ ID NO: 53, SEQ ID NO: 60, SEQ ID NO: 61, SEQ ID NO: 62, SEQ ID NO: 63, SEQ ID NO: 64, or SEQ ID NO: 65.

32. The activity-inducible fusion protein of claim 23, wherein the extracellular component further comprises a spacer region.

33. The activity-inducible fusion protein of claim 32, wherein the spacer region comprises an IgG4 hinge.

34. The activity-inducible fusion protein of claim 23, wherein the intracellular signaling domain comprises a CD3Z signaling domain.

35. The activity-inducible fusion protein of claim 23, wherein the intracellular signaling domain comprises a 4-1BB signaling domain.

36. The activity-inducible fusion protein of claim 23, wherein the intracellular signaling domain comprises a CD3Z signaling domain and a 4-1BB signaling domain.

37. The activity-inducible fusion protein of claim 23, wherein the transmembrane domain comprises a CD28 transmembrane domain.

38. The activity-inducible fusion protein of claim 23, wherein the intracellular signaling domain further comprises a co-stimulatory immune molecule selected from 4-1BB, OX40, CD40, CD30, CD27, DR3, SLAMF1, ICOS, GITR, CD25, CD28, CD79A, CD79B, CD226, CARD11, DAP10, DAP12, DR3, FcRα, FcRβ, FcRγ, Fyn, Lck, LAT, LRP, LIGHT, NKG2D, NOTCH1, NOTCH2, NOTCH3, NOTCH4, ROR2, Ryk, Slp76, pTα, TCRα, TCRβ, TIM1, TRIM, Zap70, an PTCH2.

39. A nucleotide encoding an activity-inducible fusion protein of claim 1.

40. A cell genetically modified to express an activity-inducible fusion protein of claim 1.

41. The cell of claim 40, genetically modified to express at least two types of an activity-inducible fusion protein of claim 1, wherein the two types have different transcription factors and different hsp90 binding domains that bind different drug molecules.

42. The cell of claim 40, wherein the cell is a T cell or a natural killer (NK) cell.

43. The cell of claim 42, wherein the T cell is a CD4+ or a CD8+ T cell.

44. The cell of claim 40, wherein the cell is an induced pluripotent stem cell (iPSC), a tumor-infiltrating lymphocyte (TIL), a marrow-infiltrating lymphocyte (MIL), a natural killer T cell (NKT), a mucosal-associated invariant T (MAIT) cell, a dendritic cell, a monocyte or a macrophage.

45. A system for altering the activation state of an immune cell comprising:
the cell of claim 40; and
the drug molecule.

46. The system of claim 45, wherein the cell is ex vivo or in vivo.

47. The system of claim 45, formulated for administration to a subject.

48. The system of claim 47, wherein the formulated system comprises
(j) ex vivo manufactured cells expressing the activity-inducible fusion protein formulated into a pharmaceutically acceptable carrier to create a modified formulation; and/or
(ii) cell-targeted viral vectors and/or cell-targeted nanoparticles formulated into a pharmaceutically acceptable carrier to create a modifying formulation wherein the cell-targeted viral vectors and/or cell-targeted nanoparticles comprise gene-modifying components that result in expression of the activity-inducible fusion protein in vivo by the targeted cell following administration; and
(iii) the drug molecule formulated into a pharmaceutically acceptable carrier to create a drug composition.

49. A method of treating a subject in need thereof comprising administering a system of claim 45 to the subject, thereby treating the subject.

50. The method of claim 49, wherein the administering comprises administering the modified formulation and the drug composition.

51. The method of claim 49, wherein the administering comprises administering the modifying formulation and the drug composition.

52. The method of claim 49, wherein the method further comprises stopping administration of the drug molecule to reduce a side effect of system administration.

53. The method of claim 49, wherein the method further comprises stopping administration of the drug molecule when the subject is no longer in need thereof.

54. The method of claim 49, wherein the system comprises at least two types of an activity-inducible fusion protein, wherein the at least two types comprise (1) a first activity-inducible fusion protein comprising a first hsp90 binding domain that binds a first drug molecule and a (2) second activity-inducible fusion protein comprising a second hsp90 binding domain that binds a second drug molecule, wherein the first hsp90 binding domain and second hsp90 binding domain are different.

55. The method of claim 54, comprising administering at least the first drug molecule and the second drug molecule, wherein the first and second drug molecules are different.

56. The method of claim 55, wherein the method further comprises stopping administration of the first drug molecule or second drug molecule.

57. The method of claim 55, wherein the method further comprises stopping administration of the first drug molecule and the second drug molecule.

58. The method of claim 54, wherein the first activity-inducible fusion protein comprises a first transcription factor and the second activity-inducible fusion protin comprises a second transcription factor, wherein the first transcription factor and second transcription factor are different.

59. The method of claim 49, wherein the subject is in need thereof due to cancer or an infection.

60. The method of claim 59, wherein the cancer is bladder cancer, head and neck cancer, breast cancer, colon cancer, kidney cancer, liver cancer, lung cancer, ovarian cancer, prostate cancer, pancreatic cancer, stomach cancer, cervical cancer, thyroid cancer skin cancer, hematopoietic cancer of a lymphoid lineage, hematopoietic cancer of myeloid lineage, neuroblastoma, glioma; tumors of the central and peripheral nervous system, comprising astrocytoma, neuroblastoma, or glioma.

61. The method of claim 59, wherein the cancer is acute lymphoblastic leukemia (ALL), acute myeloid leukemia (AML), or a brain cancer.

62. The method of claim 59, wherein the infection is a bacterial, viral, fungal, parasitic, or arthropod infection.
